(19) United States
(12) Patent Application Publication
(10) Pub. No.: US 2025/0331876 A1
(43) Pub. Date: Oct. 30, 2025

(54) UNIDIRECTIONAL CONTINUOUS LOOP HYBRID CYCLIC ASPIRATION SYSTEM

(71) Applicant: NEURAVI LIMITED, GALWAY (IE)

(72) Inventors: ALAN CARNEY, GALWAY (IE); DARRAGH STAPLETON, GALWAY (IE); DAVID VALE, BARNA (IE)

(21) Appl. No.: 19/262,367

(22) Filed: Jul. 8, 2025

Related U.S. Application Data
(63) Continuation-in-part of application No. 18/441,616, filed on Feb. 14, 2024.
(60) Provisional application No. 63/447,506, filed on Feb. 22, 2023.
(60) Provisional application No. 63/674,423, filed on Jul. 23, 2024.

(51) Int. Cl. A61B 17/22; A61B 17/00; A61M 1/00

(52) U.S. Cl. CPC A61B 17/22; A61B 17/00234; A61M 1/75; A61B 2017/00238; A61B 2017/00561; A61B 2017/22079; A61M 2205/3331; A61M 2210/12

(57) ABSTRACT

Unidirectional continuous loop hybrid cyclic aspiration system including a vacuum pump and associated vacuum collection vessel connected to a liquid reservoir open to atmosphere; inlet tubing having defined therein a vacuum pressure lumen and a positive pressure lumen; an aspiration catheter in fluid communication to the inlet tubing; and a positive pressure pulse generator mechanism intermittently radially compressing the positive pressure lumen creating a positive pressure pulse; a vacuum pressure gating device controlling passage through the vacuum lumen; and a liquid replenishing gating device controlling passage of the liquid from the liquid reservoir open to the atmosphere replenishing the aspirated into the vacuum collection vessel. Travel of the collectable fluid in the system is: unidirectionally (in a proximal direction) through the vacuum pressure lumen during a vacuum pressure interval; and unidirectionally (in a distal direction) through the positive pressure lumen during the positive pressure interval.

### FIELD

The present disclosure generally relates to a system and method used during thrombectomy procedures for the capture and removal of occlusions or clots. Specifically, the present disclosure relates to a hybrid cyclic aspiration system for the capture and removal of occlusions or clots in a vessel where the cyclic aspiration pressure waveform includes intermittent cyclic intervals of vacuum pressure (i.e., below atmospheric pressure) and positive pressure (i.e., higher than vacuum pressure, possibly higher than atmospheric pressure). The hybrid cyclic aspiration system produces the cyclic aspiration waveform using a positive pressure pulse generator mechanism externally compressing a section of inlet tubing disposed in fluid communication between a vacuum pump and aspiration catheter, wherein the inlet tubing has a vacuum pressure lumen and separate therefrom a positive pressure lumen for unidirectional travel of collected fluid in each lumen in opposite directions of one another forming a continuous pathway or loop.

### CROSS-REFERENCE TO RELATED APPLICATION

The present application claims the benefit of U.S. Provisional Patent Application No. 63/674,423, filed Jul. 23, 2024; and the present application is a continuation-in-part of U.S. patent application Ser. No. 18/441,616, filed Feb. 14, 2024 (U.S. Publication No. 2024/0277357), which claims the benefit of U.S. Provisional Application No. 63/447,506, filed Feb. 22, 2023, the entirety of which are hereby incorporated by reference.

### BACKGROUND

Pulsatile or cyclic aspiration applies a cyclic pressure waveform of intermittent cyclic minimum/low/vacuum/aspiration pressure and maximum/peak/high/positive pressure. During cycles under the minimum/low/vacuum/aspiration pressure the clot is drawn in the proximal direction and captured at the distal tip/end of the aspiration catheter, whereas during cycles of maximum/peak/high/positive pressure the clot is pushed in the distal direction. When utilizing pulsatile or cyclic aspiration during the capture and removal of the clot it is desirable to maximize the cycling frequency of the cyclic pressure waveform and thus maximize clot vibration thereby optimizing aspiration performance. One key challenge in maximizing the cycling frequency is a particular response time required for mechanical actuation of each active component limiting an extent to which the cycling frequency may be increased. Complex conventional systems for maximizing cycling frequency have many active components each required to await their response times before being activated to maintain normal operation. Accordingly, in complex systems with many active components the extent to which the cycling frequency may be maximized is undesirably curtailed.

Efficiency of capture of the clot depends on optimizing consistency and continuity in the cyclic aspiration pressure waveform produced at the distal tip of the aspiration catheter. Minimizing formation of bubbles in the collected fluid is a factor in optimizing consistency and continuity in the generated cyclic aspiration pressure waveform. In addition, efficiency of capture is also impacted by the risk of clogging of the clots captured using a cyclic aspiration system.

It is therefore desirable to develop an improved hybrid cyclic aspiration system utilizing as few active components as possible with an associated maximized response time to attain maximum cycling frequency while also minimizing dampening or decay of the positive pressure wave as well as the additional benefit of reducing the overall cost of manufacture. Still further desirable is to develop an improved hybrid cyclic aspiration system optimizing consistency and continuity in the cyclic aspiration pressure waveform produced at the distal tip of the aspiration catheter by minimizing occurrence of formed bubbles in the collected fluid while preventing, or minimizing, risk of clogging by captured clots.

### SUMMARY

An aspect of the present disclosure relates to a pulsatile or cyclic aspiration system producing a cyclic aspiration pressure waveform of intermittent cyclic intervals of vacuum pressure below atmospheric pressure and positive pressure higher than vacuum pressure (higher than vacuum pressure, possibly higher than atmospheric pressure) using as few active components as possible with an associated maximized response time to attain maximum cycling frequency while also minimizing dampening or decay of the positive pressure wave as well as the additional benefit of reducing the overall cost of manufacture.

Another aspect of the present disclosure is directed to a cyclic aspiration system producing a cyclic aspiration pressure waveform using a vacuum pump connected in fluid communication with an aspiration catheter via a conduit (e.g., inlet tubing, housing, or a rotating hemostatic valve) having a positive pressure pulse generator mechanism and associated at least one gating device.

In yet another aspect of the present disclosure is directed to a cyclic aspiration system producing a cyclic aspiration pressure waveform using a vacuum pump connected in fluid communication with an aspiration catheter via a conduit (e.g., inlet tubing, housing, or a rotating hemostatic valve) having a positive pressure pulse generator mechanism and associated at least one gating device, wherein the at least one gating device includes at least one actuator component arranged externally of the conduit, not contaminated by blood, reusable, and separable from non-actuator components (e.g., conduit and components disposed therein) contaminated by blood and discardable after a single use.

While still another aspect of the present disclosure is directed to a hybrid cyclic aspiration system in which formed bubbles that otherwise would dampen or decay the cyclic aspiration pressure waveform unidirectionally travel through the inlet tubing as a continuous loop or pathway and exit from the inlet tubing when aspirated as part of the collected fluid into a vacuum collection vessel while also replenishing back into the inlet tubing liquid depleted during aspiration.

Yet still another aspect of the present disclosure is directed to a hybrid cyclic aspiration system in which the liquid reservoir open to the atmosphere is refillable with the recirculated aspirated collected fluid stored in the vacuum collection vessel, wherein the formed bubbles in the collected fluid escape in the atmosphere leaving behind degassed liquid free of the formed bubbles.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices, by way of example only, not by way of limitation.

FIG. 1A diagrammatically depicts an example of a vented cyclic aspiration system in which the cyclic aspiration pressure waveform is created using a vacuum pump connected in fluid communication to the aspiration catheter by inlet tubing vented to a liquid reservoir subject to atmospheric pressure;

FIG. 1B diagrammatically depicts another example of a vented cyclic aspiration system in which the cyclic aspiration pressure waveform is created using a vacuum pump connected in fluid communication to the aspiration catheter by inlet tubing vented to a closed reservoir pressurized by displacing an internal plunger;

FIG. 1C diagrammatically depicts still another example of a vented cyclic aspiration system in which the cyclic aspiration pressure waveform is created using a vacuum pump connected in fluid communication to the aspiration catheter by inlet tubing vented to a pressurized saline bag;

FIG. 1D diagrammatically depicts yet another example of a vented cyclic aspiration system in which the cyclic aspiration pressure waveform is created using a vacuum pump connected in fluid communication to the aspiration catheter by inlet tubing vented to a pressurized accumulator;

FIG. 2 diagrammatically depicts an example of a non-vented cyclic aspiration system in which the cyclic aspiration pressure waveform is created using a vacuum pump connected in fluid communication to the aspiration catheter by flexible inlet tubing a section of which is externally compressed by a plunger to produce the positive pressure pulse;

FIG. 3A diagrammatically depicts an example of a hybrid cyclic aspiration system in which the cyclic aspiration pressure waveform is created using a vacuum pump connected in fluid communication to the aspiration catheter by inlet tubing a section of which is externally compressed by a plunger to produce the positive pressure pulse while the system is also vented to a liquid reservoir subject to atmospheric pressure to minimize dampening or decay of the pressure wave;

FIG. 3B is an axial cross-section of a solid support illustrating heterogeneous bubble nucleation during different stages of bubble growth or formation;

FIG. 3C depicts a section of inlet tubing illustrating how a larger bubble size (without combating bubble growth) impedes, disrupts, or blocks the positive pressure wave resulting in dampening of the pressure wave;

FIG. 3D depicts a section of inlet tubing illustrating how a smaller bubble size (reduced bubble growth) does not impede, disrupt, or block the pressure wave thereby minimizing dampening of the pressure wave;

FIG. 3E is a representative comparison of the bubble size and gas pressure;

FIG. 3F is an exemplary graphical representation of the pressure waveform having a reduced or lower positive pressure pulse to eliminate or minimize bubbles in the system;

FIG. 3G is an exemplary graphical representation of the pressure waveform having a period of constant vacuum to eliminate or minimize bubbles in the system;

FIG. 3H is an exemplary graphical representation of a two-stage positive pressure plunge using a displaceable plunger, wherein the two-stage plunge includes a pre-plunge, a hold period, and a main plunge;

FIG. 3I diagrammatically depicts another example of a hybrid cyclic aspiration system in which the cyclic aspiration pressure waveform is created using a vacuum pump connected in fluid communication to the aspiration catheter by inlet tubing a section of which is externally compressed by a plunger to produce the positive pressure pulse while the system is also intermittently vented to a fluctuation reservoir open to atmospheric pressure to minimize dampening or decay of the pressure wave;

FIG. 3J is an exemplary graphical representation of the cyclic aspiration pressure waveform produced by the hybrid cyclic aspiration system of FIG. 3I and the associated states of the gating devices;

FIG. 3K diagrammatically depicts yet another example of a hybrid cyclic aspiration system in which the cyclic aspiration pressure waveform is created using a vacuum pump connected in fluid communication to the aspiration catheter by inlet tubing a section of which is externally compressed by a plunger to produce the positive pressure pulse while the system is also vented to a fluctuation reservoir open to atmospheric pressure to minimize dampening or decay of the pressure wave;

FIG. 3L is an exemplary graphical representation of the cyclic aspiration pressure waveform produced by the hybrid cyclic aspiration system of FIG. 3K and the associated states of the gating devices;

FIG. 3M diagrammatically depicts still another example of a hybrid cyclic aspiration system in which the cyclic aspiration pressure waveform is created using a vacuum pump connected in fluid communication to the aspiration catheter by inlet tubing a section of which is externally compressed by a plunger to produce the positive pressure pulse while the system is also vented to atmospheric pressure to minimizes dampening or decay of the pressure wave;

FIG. 3N is an exemplary graphical representation of the cyclic aspiration pressure waveform produced by the hybrid cyclic aspiration system of FIG. 3M and the associated states of the gating devices;

FIG. 4 diagrammatically depicts while still another example of a hybrid cyclic aspiration system in which the cyclic aspiration pressure waveform is created using a vacuum pump connected in fluid communication to the aspiration catheter by inlet tubing a section of which is externally compressed by a plunger to produce the positive pressure pulse while the system is also vented to atmospheric pressure to minimize dampening or decay of the pressure wave while also including a surge tank to optimize hammer spikes;

FIG. 5A diagrammatically depicts an example self-prepping hybrid cyclic aspiration system, wherein filling of the liquid reservoir with saline is automatized using the vacuum pump;

FIG. 5B diagrammatically depicts another example self-prepping hybrid cyclic aspiration system, wherein filling of the liquid reservoir with saline is automatized using the vacuum pump;

FIG. 5C diagrammatically depicts still another example self-prepping hybrid cyclic aspiration system, wherein filling of the liquid reservoir with saline is automatized using the vacuum pump;

FIG. 6A diagrammatically depicts an example vented cyclic aspiration system double vented to two different liquid reservoirs open to atmospheric pressure; one liquid reservoir produces a non-heightened positive pressure pulse moving the clot distally within the aspiration catheter without expulsion from the distal tip, while the other liquid reservoir produces an aggressive or heightened positive pressure pulse expelling the clot from the distal tip of the aspiration catheter;

FIG. 6B is an exemplary graphical representation pressure waveform over time of four cycles of non-heightened positive pressure pulses followed by four cycles of aggressive or heightened positive pressure pulses;

FIG. 6C is an exemplary graphical representation of optimum range of amplitude or peak positive pressure pulse of the cyclic aspiration system;

FIG. 7A diagrammatically depicts an example vented cyclic aspiration system utilizing a rotating reciprocating mechanism (e.g., a scotch yoke mechanism) acting as a single gating device for controlling passage through respective vacuum inlet tubing connected in fluid communication with a vacuum pump and positive pressure inlet tubing vented to atmospheric pressure;

FIG. 7B depicts a rotational stage of the scotch yoke mechanism with the external shaft at a first maximum position constricting passage of the vacuum pressure through the vacuum inlet tubing, while allowing unblocked passage of the positive pressure through the positive pressure inlet tubing producing a positive pressure pulse;

FIG. 7C depicts a rotational stage of the scotch yoke mechanism with the external shaft at an equilibrium or midway position between the first and second maximum positions constricting passage of the vacuum pressure through the vacuum inlet tubing and positive pressure through the positive pressure inlet tubing allowing dissipation of the pressurized fluid in the system due to viscous losses;

FIG. 7D depicts a rotational stage of the scotch yoke mechanism with the external shaft at a second maximum position constricting passage of the positive pressure through the positive pressure inlet tubing, while allowing unblocked passage of the vacuum pressure through the vacuum pressure inlet tubing;

FIG. 7E depicts an enlarged view of the motor for controlling displacement of a slotted rotating pin within a channel defined in the positive pressure inlet tubing acting as a gating device for varying the amplitude of the positive pressure pulse; wherein the slotted rotating pin is shown in a fully advanced state occluding or blocking passage therethrough of the positive pressure;

FIG. 7F depicts an enlarged view of the motor for controlling displacement of a slotted rotating pin within a channel defined in the positive pressure inlet tubing acting as a gating device for varying the amplitude of the positive pressure pulse; wherein the slotted rotating pin is shown in a fully retracted state allowing unrestricted or maximum passage therethrough of the positive pressure;

FIG. 8A diagrammatically depicts a top view of an example of a vented cyclic aspiration system in which the cyclic aspiration pressure waveform is created using a rotating hemostatic valve having a vacuum pressure inlet port connected in fluid communication to a vacuum pump and a positive pressure inlet port vented to a saline bag; a rotating reciprocating mechanism displacing pins acting as gating devices within respective holes defined in a wall of each of the vacuum pressure inlet port and the positive pressure inlet port;

FIG. 8B is a top perspective view of a rotatable single actuator wheel illustrating the undulating recessed and planar regions of the rotating reciprocating mechanism in the vented cyclic aspiration system of FIG. 8A;

FIG. 8C is a side view of the rotatable single actuator wheel of the rotating reciprocating mechanism in the vented cyclic aspiration system of FIG. 8A;

FIG. 9A diagrammatically depicts a side view of an example of a non-vented cyclic aspiration system in which the cyclic aspiration pressure waveform is created using a rotating hemostatic valve having a vacuum pressure inlet port connected in fluid communication to a vacuum pump; a rotating reciprocating mechanism displacing a single pin acting as gating device within a respective hole defined in a wall of the rotating hemostatic valve and secured to a displaceable internal plunger for generating the positive pressure pulse; the pin and plunger are shown in a fully retracted state allowing unrestricted maximum passage therethrough of the vacuum pressure generated by the vacuum pump;

FIG. 9B diagrammatically depicts a side view of the example non-vented cyclic aspiration system of FIG. 9A using the rotating hemostatic valve, wherein the pin and plunger are shown in a fully advanced state with the pin acting as a valve constricting passage of vacuum pressure therethrough while the plunger reduces the internal volume displacing the fluid collected therein thereby generating the positive pressure pulse (i.e., injection of positive pressure);

FIG. 10A illustrates an exemplary gating device actuated by externally arranged electromagnets for use with any of the exemplary cyclic aspiration systems disclosed herein utilizing inlet tubing disposed in fluid communication between the vacuum pump and proximal hub attached to the aspiration catheter; wherein the exemplary gating device is illustrated without the internal displaceable ball;

FIG. 10B illustrates the exemplary gating device of FIG. 10A including the internal displaceable ball within the inlet tubing shown in an open state allowing unrestricted maximum passage therethrough;

FIG. 10C illustrates the exemplary gating device of FIG. 10A including the internal displaceable ball within the inlet tubing shown in a closed state blocking or occluding passage therethrough;

FIG. 10D illustrates the exemplary gating device of FIG. 10A differentiating among the actuator component (e.g., electromagnet) arranged externally of the inlet tubing, not contaminated by blood, and reusable which is separable from the non-actuator components (e.g., the housing, the inlet tubing and the internal displaceable ball), contaminated by blood, and discarded after a single use;

FIG. 11A illustrates another gating device actuated by an externally arranged electromagnet for use with any of the exemplary cyclic aspiration systems disclosed herein utilizing inlet tubing disposed in fluid communication between the vacuum pump and proximal hub attached to the aspiration catheter; wherein the exemplary gating device is illustrated without the externally arranged electromagnet;

FIG. 11B illustrates the exemplary gating device of FIG. 11A including the spring in a non-compressed fully extended state maintaining the internal displaceable ball at the tapered end of the inlet tubing while in a closed state blocking or occluding passage therethrough;

FIG. 11C illustrates the exemplary gating device of FIG. 11A including the internal displaceable ball drawn by the electromagnet to the wider end of the inlet tubing thereby compressing the spring while in an open state allowing unrestricted maximum passage therethrough;

FIG. 11D illustrates the exemplary gating device of FIG. 11A differentiating among the actuator component (electromagnet) arranged externally of the inlet tubing, not contaminated by blood, and reusable which is separable from the non-actuator components (e.g., the inlet tubing, the spring, and the internal displaceable ball), contaminated by blood, and discarded after a single use;

FIG. 11E illustrates yet another gating device actuated by externally arranged electromagnets for use with any of the exemplary cyclic aspiration systems disclosed herein utilizing inlet tubing disposed in fluid communication between the vacuum pump and proximal hub attached to the aspiration catheter; wherein the exemplary gating device is illustrated without the externally arranged electromagnets;

FIG. 11F illustrates the exemplary gating device of FIG. 11E with the internal displaceable ball drawn by the distal electromagnet towards the tapered end of the inlet tubing while in a closed state blocking or occluding passage therethrough;

FIG. 11G illustrates the exemplary gating device of FIG. 11E with the internal displaceable ball drawn by the proximal electromagnet towards the wider end of the inlet tubing while in an open state allowing unrestricted maximum passage therethrough;

FIG. 11H illustrates the exemplary gating device of FIG. 11E differentiating among the actuator components (electromagnets) arranged externally of the inlet tubing, not contaminated by blood, and reusable which are separable from the non-actuator components (e.g., the inlet tubing, the spring, and the internal displaceable ball), contaminated by blood, and discarded after a single use;

FIG. 12A diagrammatically depicts an example hybrid cyclic aspiration system wherein the inlet tubing at the vacuum pump is higher relative to the inlet tubing at the positive pressure pulse generator mechanism (e.g., plunger) with a vertical offset section of the inlet tubing therebetween forming a mini reservoir;

FIG. 12B diagrammatically depicts another example hybrid cyclic aspiration system wherein the inlet tubing at the vacuum pump is higher relative to the inlet tubing at the positive pressure pulse generator mechanism (e.g., plunger) with a vertical offset section of the inlet tubing therebetween forming a petite reservoir smaller in volume relative to that of the mini reservoir in FIG. 12A;

FIG. 12C diagrammatically depicts still another example hybrid cyclic aspiration system wherein the inlet tubing at the vacuum pump is higher relative to the inlet tubing at the positive pressure pulse generator mechanism (e.g., plunger), wherein the entire hybrid cyclic aspiration system is inclined either vertically/perpendicularly or at an angle relative to the inlet tubing at the vacuum pump;

FIG. 13A is an example schematic representation of a unidirectional continuous loop hybrid cyclic aspiration system wherein inlet tubing is a single inlet tube having a vacuum lumen and a separate positive pressure lumen arranged side-by-side in the longitudinal/axial direction defined therein; the collected fluid in the system travels only unidirectionally in a distal direction through the positive pressure lumen during the positive pressure interval and only unidirectionally in a proximal direction through the vacuum pressure lumen during the vacuum pressure interval, and wherein turnaround travel of the collected liquid from the positive pressure lumen into the vacuum pressure lumen occurs in a clearance space defined in a distal section of the single inlet tube forming a continuous loop or pathway;

FIG. 13B is an enlarged view of the interface between the catheter hub and the distal section of the single inlet tube in FIG. 13A;

FIG. 13C is a radial cross-sectional view along lines 13C-13C through the single inlet tube in FIG. 13B depicting the side-by-side arrangement in the longitudinal/axial direction of the vacuum pressure lumen and the positive pressure lumen;

FIG. 13D is a radial cross-sectional view along lines 13D-13D through the clearance space in a distal section of the single inlet tube in FIG. 13B;

FIG. 14A is another example unidirectional continuous loop hybrid cyclic aspiration system in accordance with the present disclosure wherein the inlet tubing is a single inlet tube having a vacuum lumen and separate positive pressure lumen arranged concentrically defined therein; the fluid collected in the system travels only unidirectionally in a distal direction through the positive pressure lumen during the positive pressure interval and only unidirectionally in a proximal direction through the vacuum pressure lumen during the vacuum pressure interval, and wherein turnaround travel of the collected liquid from the positive pressure lumen into the vacuum pressure lumen occurs in a clearance space defined in a distal section of the single inlet tube forming a continuous loop or pathway;

FIG. 14B is an enlarged view of the interface between the catheter hub and the distal section of the single inlet tube in FIG. 14A;

FIG. 14C is a radial cross-sectional view along lines 14C-14C through the single inlet tube in FIG. 14B depicting the concentric arrangement of the vacuum pressure lumen and the positive pressure lumen;

FIG. 14D is a radial cross-sectional view along lines 14D-14D through the clearance space in a distal section of the inlet tube in FIG. 14B;

FIG. 15A is another example unidirectional continuous loop hybrid cyclic aspiration system in accordance with the present disclosure wherein the inlet tubing is a single inlet tube having a vacuum lumen and separate positive pressure lumen arranged eccentrically defined therein; the fluid collected in the system travels only unidirectionally in a distal direction through the positive pressure lumen in response to creating of the positive pressure pulse during the positive pressure interval and only unidirectionally in a proximal direction through the vacuum pressure lumen during the vacuum pressure interval, and wherein turnaround travel of the collected fluid from the positive pressure lumen into the vacuum pressure lumen occurs in a distal section of the single inlet tube forming a continuous loop or pathway;

FIG. 15B is an enlarged interface between the catheter hub and the distal section of the single inlet tube in FIG. 15A;

FIG. 15C is a radial cross-sectional view along lines 15C-15C through the single inlet tube in FIG. 15B depicting the eccentric arrangement of the vacuum pressure lumen and the positive pressure lumen;

FIG. 15D is a radial cross-sectional view along lines 15D-15D through the clearance space in a distal section of the single inlet tube in FIG. 15B;

FIG. 16A is an example unidirectional continuous loop hybrid cyclic aspiration system in accordance with the present disclosure wherein the inlet tubing is a single inlet tube having a vacuum lumen and separate positive pressure lumen arranged side-by-side in the longitudinal/axial direction defined therein; the fluid collected in the system travels only unidirectionally in a distal direction through the positive pressure lumen in response to the positive pressure pulse generator mechanism creating of the positive pressure pulse during the positive pressure interval and only unidirectionally in a proximal direction through the vacuum pressure lumen during the vacuum pressure interval, and wherein turnaround travel of the collected fluid from the positive pressure lumen into the vacuum pressure lumen occurs in a proximal section of the catheter hub forming a continuous loop or pathway;

FIG. 16B is an enlarged interface between the catheter hub and the distal section of the single inlet tube in FIG. 16A;

FIG. 16C is a radial cross-sectional view along lines 16C-16C through the single inlet tube in FIG. 16B depicting the side-by-side arrangement in the longitudinal/axial direction of the vacuum pressure lumen and the positive pressure lumen;

FIG. 17A is another example unidirectional continuous loop hybrid cyclic aspiration system in accordance with the present disclosure wherein the inlet tubing is a single inlet tube having a vacuum lumen and separate positive pressure lumen arranged concentrically defined therein; the fluid collected in the system travels only unidirectionally in a distal direction through the positive pressure lumen in response to creating of the positive pressure pulse during the positive pressure interval and only unidirectionally in a proximal direction through the vacuum pressure lumen during the vacuum pressure interval, and wherein turnaround travel of the collected fluid from the positive pressure lumen into the vacuum pressure lumen occurs in a proximal section of the catheter hub forming a continuous loop or pathway;

FIG. 17B is an enlarged interface between the catheter hub and the distal section of the single inlet tube in FIG. 17A;

FIG. 17C is a radial cross-sectional view along lines 17C-17C through the single inlet tube in FIG. 17B depicting the concentric arrangement of the vacuum pressure lumen and the positive pressure lumen;

FIG. 18A is another example unidirectional continuous loop hybrid cyclic aspiration system in accordance with the present disclosure wherein the inlet tubing is a single inlet tube having a vacuum lumen and separate positive pressure lumen arranged eccentrically defined therein; the fluid collected in the system travels only unidirectionally in a distal direction through the positive pressure lumen in response to creating of the positive pressure pulse during the positive pressure interval and only unidirectionally in a proximal direction through the vacuum pressure lumen during the vacuum pressure interval, and wherein turnaround travel of the collected fluid from the positive pressure lumen into the vacuum pressure lumen occurs in a proximal section of the catheter hub forming a continuous loop or pathway;

FIG. 18B is an enlarged interface between the catheter hub and the distal section of the single inlet tube in FIG. 18A;

FIG. 18C is a radial cross-sectional view along lines 18C-18C through the single inlet tube in FIG. 18B depicting the eccentric arrangement of the vacuum pressure lumen and the positive pressure lumen;

FIG. 19 is an example graphical representation of the cyclic aspiration pressure waveform over time produced at the distal tip of the aspiration catheter for any of the example hybrid cyclic aspiration systems in FIGS. 13A, 14A, 15A, 16A, 17A, 18A and 25A;

FIG. 20 is an exemplary graphical representation of pressure over time depicting the three stages of operation (e.g., Stage 1—positive pressure interval; Stage 2—vacuum pressure interval; Stage 3—liquid replenishment interval) for any of the example hybrid cyclic aspiration systems in FIGS. 13A, 14A, 15A, 16A, 17A, 18A & 25A; wherein Stage 3—liquid replenishment interval occurs once every cycle;

FIG. 21 is an exemplary alternative graphical representation of pressure over time depicting the three stages of operation (e.g., Stage 1—positive pressure interval; Stage 2—vacuum pressure interval; Stage 3—liquid replenishment interval) for any of the example hybrid cyclic aspiration systems in FIGS. 13A, 14A, 15A, 16A, 17A, 18A and 25A; wherein Stage 3—liquid replenishment interval occurs once every N cycles, wherein N is 3 in the example of FIG. 21; the exemplary duration of the replenishment interval in FIG. 21 is relatively brief (i.e., before being sustained at a pressure plateau);

FIG. 22 is an exemplary alternative graphical representation of pressure over time depicting the three stages of operation (e.g., Stage 1—positive pressure interval; Stage 2—vacuum pressure interval; Stage 3—liquid replenishment interval) for any of the example hybrid cyclic aspiration systems in FIGS. 13A, 14A, 15A, 16A, 17A and 18A; wherein Stage 3—liquid replenishment interval occurs once every N cycles, wherein N is 3 in the example of FIG. 22; the exemplary duration of the replenishment interval in FIG. 22 is sustained (i.e., extended in duration at a pressure plateau) in comparison to that of the relatively brief replenishment interval in FIG. 21; and

FIG. 23 is an exemplary flow chart of operation for any of the example hybrid cyclic aspiration systems in FIGS. 13A, 14A, 15A, 16A, 17A, 18A & 25A including a periodic interval of replenishing liquid in the cyclic aspiration system;

FIG. 24 is an exemplary flow chart of operation for refilling of the liquid in the liquid reservoir open to the atmosphere for any of the example hybrid cyclic aspiration systems in FIGS. 13A, 14A, 15A, 16A, 17, 18A & 25A;

FIG. 25A is an example hybrid cyclic aspiration system in accordance with the present disclosure wherein the inlet tubing is two separate inlet tubes each having a single lumen defined therein; the two inlet tubes include a vacuum pressure inlet tube having defined therein a vacuum pressure lumen and a separate positive pressure inlet tube having defined therein a positive pressure lumen; the fluid collected in the system travels only unidirectionally in a distal direction through the positive pressure lumen in response to the positive pressure pulse generator mechanism creating of the positive pressure pulse during the positive pressure interval and only unidirectionally in a proximal direction through the vacuum pressure lumen during the vacuum pressure interval, and wherein turnaround travel of the collected fluid from the positive pressure lumen into the vacuum pressure lumen occurs in a proximal section of the catheter hub forming a continuous loop or pathway;

FIG. 25B is an enlarged interface between the catheter hub and the distal section of the single inlet tube in FIG. 25A; and

FIG. 25C is a radial cross-sectional view along lines 25C-25C through the single inlet tube in FIG. 25B.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

As used herein, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal. Computer readable medium can be non-transitory. Non-transitory computer-readable media include, but are not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable instructions and/or data.

As used herein, the term “computing system” is intended to include stand-alone machines or devices and/or a combination of machines, components, modules, systems, servers, processors, memory, detectors, user interfaces, computing device interfaces, network interfaces, hardware elements, software elements, firmware elements, and other computer-related units. By way of example, but not limitation, a computing system can include one or more of a general-purpose computer, a special-purpose computer, a processor, a portable electronic device, a portable electronic medical instrument, a stationary or semi-stationary electronic medical instrument, or other electronic data processing apparatus.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

The present disclosure is directed to a cyclic aspiration system for producing a cyclic aspiration pressure waveform of intermittent cyclic intervals of vacuum pressure below atmospheric pressure and positive pressure higher than vacuum pressure, possibly higher than atmospheric pressure, using a vacuum pump connected in fluid communication to the aspiration catheter via a conduit with an associated positive pressure pulse generator mechanism for intermittently cyclically producing the positive pressure pulse. The conduit may include: (i) inlet tubing (e.g., vacuum inlet tubing and/or positive pressure inlet tubing) disposed between the vacuum pump and the aspiration catheter; or (iii) a rotating hemostatic valve (RHV) connected to the aspiration catheter. Numerous non-limiting examples of various cyclic aspiration system for creating the cyclic aspiration system using a vacuum pump are illustrated and described herein. Depending on the manner or mechanism by which the positive pressure pulse is generated, the various cyclic aspiration systems producing the cyclic aspiration pressure waveform using a vacuum pump are grouped into three broad categories including: (i) vented cyclic aspiration systems; (ii) non-vented cyclic aspiration systems; and (iii) hybrid cyclic aspiration systems. In vented cyclic aspiration systems, the intermittent cyclic generation of the positive pressure pulse is realized by “venting” the conduit (e.g., inlet tubing or RHV) connecting in fluid communication the aspiration catheter to the vacuum pump to a positive pressure source at atmospheric pressure or higher. Specifically, the positive pressure source includes venting of the conduit to any one of: (i) atmospheric pressure; (ii) a liquid reservoir open to atmospheric pressure and filled with a liquid (e.g., blood and/or saline); or (iii) a pressurized closed reservoir higher than atmospheric pressure. In contrast to vented cyclic aspiration systems, non-vented cyclic aspiration systems (as the term “non-vented” suggests) are not vented to a positive pressure source having a pressure higher than vacuum pressure. Instead, generation of the positive pressure pulse in non-vented cyclic aspiration systems is accomplished using a positive pressure pulse generator mechanism associated with the conduit for intermittently cyclically reducing the internal volume displacing the fluid collected therein thereby generating a positive pressure pulse (i.e., injection of positive pressure). Lastly, hybrid cyclic aspiration systems represent a combination (i.e., hybrid) of aspects of both the vented and the non-vented cyclic aspiration systems. Generation of the positive pressure pulse in the hybrid cyclic aspiration system is accomplished using a positive pressure pulse generator mechanism associated with the conduit for intermittently cyclically reducing the internal volume displacing the fluid collected therein thereby generating a positive pressure pulse (i.e., injection of positive pressure) or using a pressurized closed reservoir higher than atmospheric pressure. In addition, the conduit in the hybrid cyclic aspiration system (as with the vented cyclic aspiration system) is also vented to a positive pressure source that includes: (i) atmospheric pressure; (ii) a liquid reservoir open to atmospheric pressure and filled with a liquid (e.g., blood and/or saline); or (iii) a pressurized closed reservoir higher than atmospheric pressure. However, venting of the conduit to the positive pressure source in the hybrid cyclic aspiration system serves a different purpose or function from the venting of the conduit to the positive pressure source in the vented cyclic aspiration system. Rather than produce the positive pressure pulse as with the vented cyclic aspiration system, the venting of the conduit to the positive pressure source in the hybrid cyclic aspiration system prevents or minimizes decay or dampening over time of the positive pressure pulse. Each of the three categories of cyclic aspiration systems are described in further detail later while referring to non-limiting examples of each.

The present disclosure is also directed to an improved cyclic aspiration system for producing a cyclic aspiration pressure waveform employing a vacuum pump using having as few active components as possible with an associated faster response time maximizing attainable cycling frequency (e.g., approximately 1 Hz to approximately 20 Hz). With this in mind, the simplistic cyclic aspiration systems in accordance with the present disclosure employ a minimum number of active components using the simplest active component, i.e., a gating device (e.g., valve). In accordance with the present disclosure, cyclic aspiration pressure waveform of vacuum pressure (i.e., below atmospheric pressure) and positive pressure (i.e., higher than vacuum pressure) is produced with a cyclic aspiration system employing a vacuum pump, a conduit, and a minimum number of gating devices as the active component to maximize attainable cycling frequency. Specifically, the cyclic aspiration system uses a gating device associated with the conduit (e.g., inlet tubing or as part of an RHV) disposed between the vacuum pump and aspiration catheter. Another concern addressed in the improved cyclic aspiration system in accordance with the present disclosure is arranging the actuator controlling or operating the gating device externally of the conduit so as to not to become contaminated by blood during use and thus reusable, while the remaining non-actuator components associated with the positive pressure pulse generator mechanism (e.g., inlet tubing, rotating hemostatic valve (RHV) and gating devices) contaminated by blood during use are inexpensive and thus discarded after a single use to prevent clogging.

Vented cyclic aspiration systems will first be described in which the positive pressure pulse is intermittently generated to produce the cyclic aspiration pressure waveform by “venting” the conduit (e.g., inlet tubing or rotating hemostatic valve (RHV)) connected in fluid communication between the vacuum pump and the aspiration catheter to a positive pressure source having a pressure at atmospheric pressure or higher. Specifically, the positive pressure source includes one of: (i) atmospheric pressure; (ii) a liquid reservoir open to atmospheric pressure and filled with a liquid (e.g., blood and/or saline); or (iii) a pressurized closed reservoir having a pressure higher than atmospheric pressure. A brief background is provided regarding the principle behind generating the positive pressure pulse by venting the conduit (e.g., inlet tubing or RHV) to a positive pressure source to generate the positive pressure pulse. When vacuum pressure is initially drawn into the conduit (e.g., inlet tubing or rotating hemostatic valve (RHV)) the pressure therein decreases while existing gases present in the system are evacuated via the vacuum thereby reducing the pressure exerted by those existing gases on the rest of the system, i.e., existing fluid (e.g., blood and/saline) therein. Eventually, all the existing gases are completely evacuated from the system at which point the pressure in the system reaches the vacuum pressure level. If this gas-evacuated region of the system is exposed (i.e., vented) to a positive pressure source gases from the higher-pressure region (e.g., atmospheric pressure or higher) will enter the gas-evacuated region of lower-pressure due to the pressure differential therebetween. Because they have mass these gases possess a certain momentum transferring energy to existing fluid in the system when suddenly stopped (i.e., impacted) by the gases thereby the fluid becoming pressurized (e.g., above atmospheric pressure if the impact is sufficiently great). In turn, the pressurized fluid briefly exerts a force on the clot captured at the distal tip/end of the aspiration catheter, dislodging it as it overcomes atmospheric pressure, before imparted energy in the pressurized fluid dissipates due to viscous losses, eventually restoring the fluid to its ambient pressure (i.e., the vacuum pressure, if a gating device to the vented positive pressure source (e.g., atmospheric pressure gating) is closed and the flow rate of the vacuum pump is sufficiently great to extract the gases from the system in such a brief period time). This momentum transfer between fluids is known as “hydraulic shock” or, more commonly, as “water hammer.” Due to the small diameter of the conduit (and thus relatively large surface area), the surface tension of the existing fluid in the system is large, and it is thus difficult for gases to penetrate the existing fluid in the system, instead pushing against the surface of the existing fluid in the system which becomes pressurized creating a positive pressure pulse (i.e., injection of positive pressure). It is further possible to control the amplitude of the pressurization (i.e., injection of positive pressure) of the existing fluid in the system by restricting or constricting the mass of the gas entering the system, thereby reducing the momentum of the gas in motion and reducing the pressure impact experienced by the fluid existing in the system. A simple way of achieving this is by constraining the diameter of the air-intake from the positive pressure source with a gating device.

Based on the above principles, several illustrative examples will now be described of vented cyclic aspiration systems producing a cyclic aspiration pressure waveform using a vacuum pump while venting the system to a positive pressure source via a gating device intermittently cyclically controlled to create a positive pressure pulse.

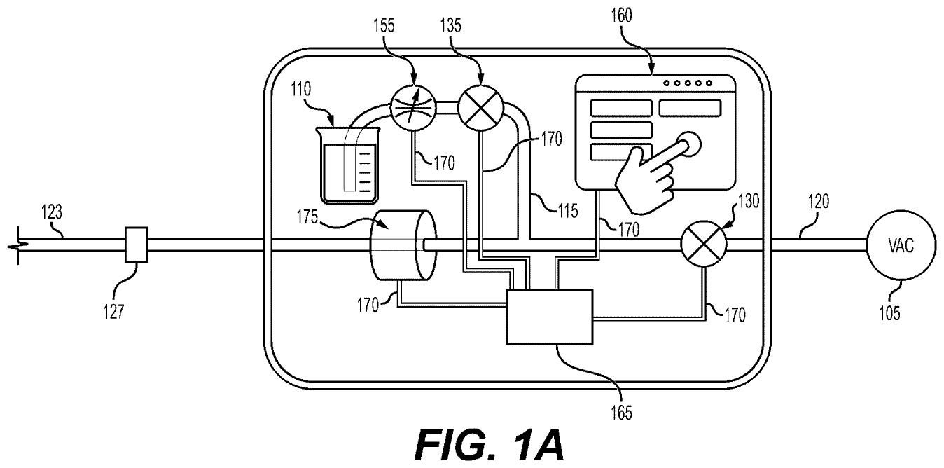

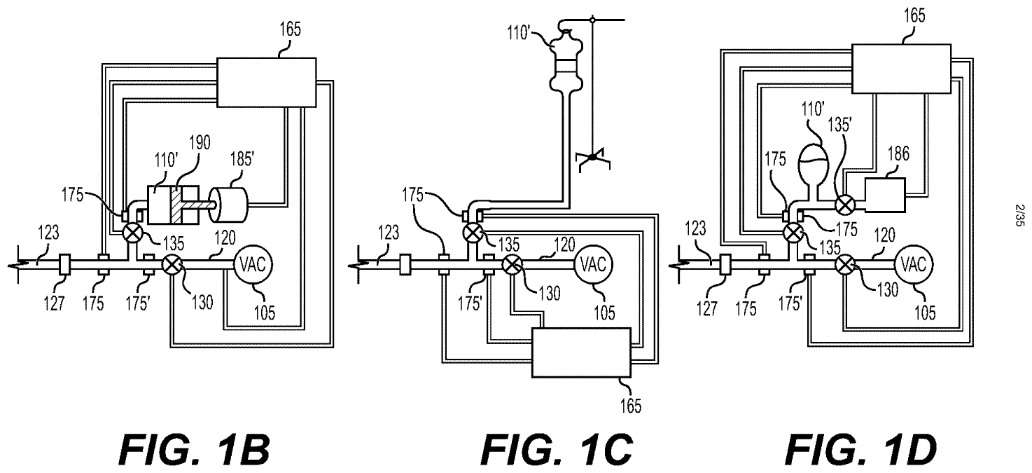

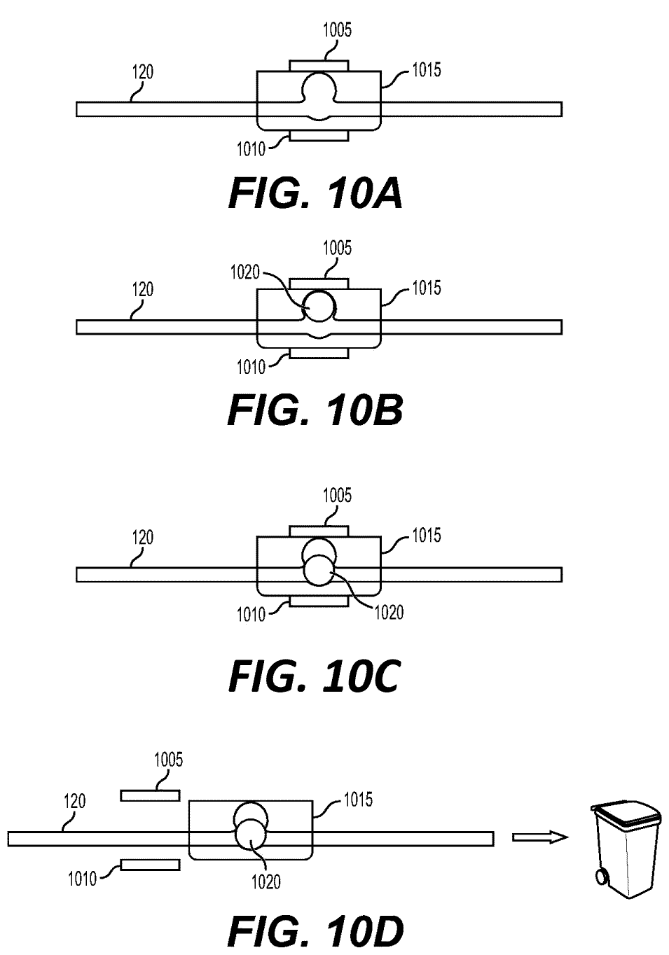

FIG. 1A depicts an exemplary vented cyclic aspiration system in accordance with the present disclosure that is vented to a liquid reservoir open to atmospheric pressure and filled with a liquid (e.g., saline and/or blood) 110 as the positive pressure source. Vacuum pressure inlet tubing 120 (i.e., vacuum line) connects in fluid communication a proximal hub 127 attached to an aspiration catheter 123 to a vacuum pump 105. Positive pressure inlet tubing 115 at a distal end is in fluid communication (e.g., via a 3-way connector) with the vacuum pressure inlet tubing 120 while an opposite proximal end is vented to the positive pressure source (e.g., liquid reservoir open to atmospheric pressure 110). In the example in FIG. 1A, the positive pressure inlet tubing 115 is vented to the liquid reservoir open to atmospheric pressure 110, however, the positive pressure inlet tubing 115 may otherwise be vented to alternative positive pressure sources such as only atmospheric pressure (i.e., without the liquid reservoir) or a closed pressurized reservoir, as described in other examples below. Referring once again to FIG. 1A, the vacuum pressure inlet tubing 120 (i.e., vacuum line) includes a first gating device 130 disposed distally of the vacuum pump 105. While the positive pressure inlet tubing 115 has associated therewith a second gating device 135 disposed distally of the liquid reservoir open to atmospheric pressure 110. Gating devices 130, 135 act as valves controlling passage therethrough. However, conventional gating devices such as internal solenoids pose significant drawbacks in that they are incapable of rapidly transitioning at rates required to attain rapid, maximum, or high cycling frequencies (e.g., approximately 1 Hz to approximately 20 Hz) and are also contaminated with blood during use hence prone to clogging. To overcome these concerns, the gating devices employed in all of the exemplary cyclic aspiration systems described herein employ inlet tubing or the RHV and are actuated, controlled, or displaced using an actuator device (e.g., linear actuator, solenoid, electromagnets, reciprocating motor, cam, rotating to reciprocating motor, etc.) arranged externally of the conduit (e.g., inlet tubing, connector, or rotating hemostatic valve (RHV)), not contaminated by blood during use, and hence reusable. Separable from the externally arranged actuator device, those remaining non-actuator components (e.g., inlet tubing, auxiliary tubing, rotating hemostatic valve, and components disposed internally thereof) for producing the positive pressure pulse are contaminated by blood and thus discarded after a single use. Several illustrative examples of such preferred gating devices are shown in FIGS. 10A-10D & 11A-11H and described in detail further below. The first and the second gating devices 130, 135 may be identical or different from one another. Amplitude of the positive pressure pulse generated in the positive pressure inlet tubing 115 is controllable via a variable restrictor 155 disposed between the liquid reservoir open to atmospheric pressure 110 and the second gating device 135 in response to pressure monitored by a pressure sensor 175 in the vacuum pressure inlet tubing 120 (i.e., vacuum line) distally of the positive pressure inlet tubing 115. Operation of the first and second gating devices 130, 135, respectively, as well as the variable restrictor 155 may be controlled by controller 165 (e.g., processor) in response to input or data received from pressure and/or flow sensor(s) 175 and/or a user interface 160, wherein all the components are electrically connected to the controller 165 via electrical wiring 170.

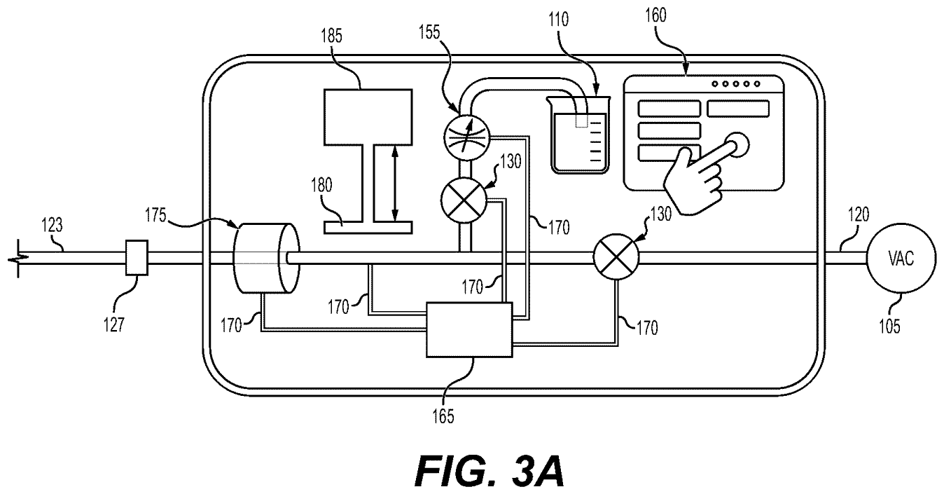

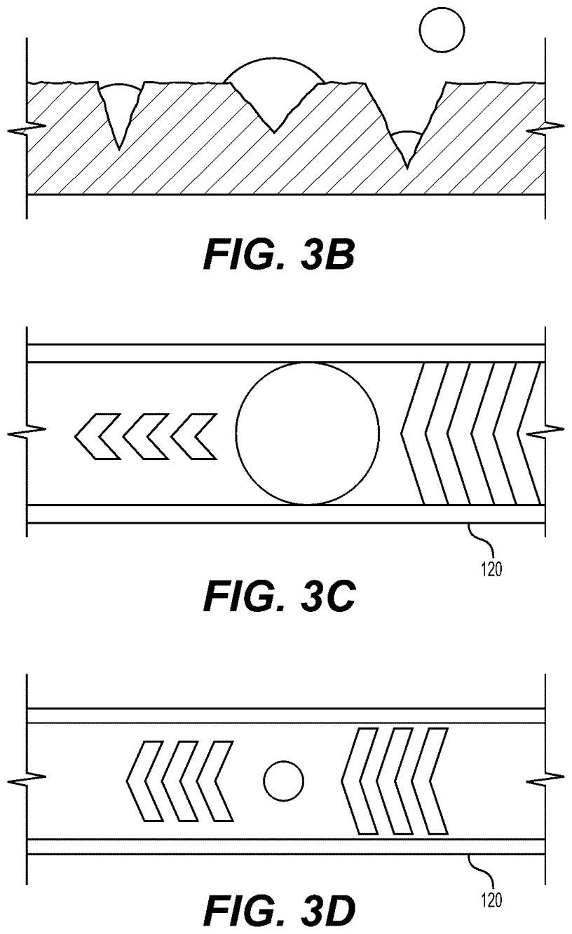

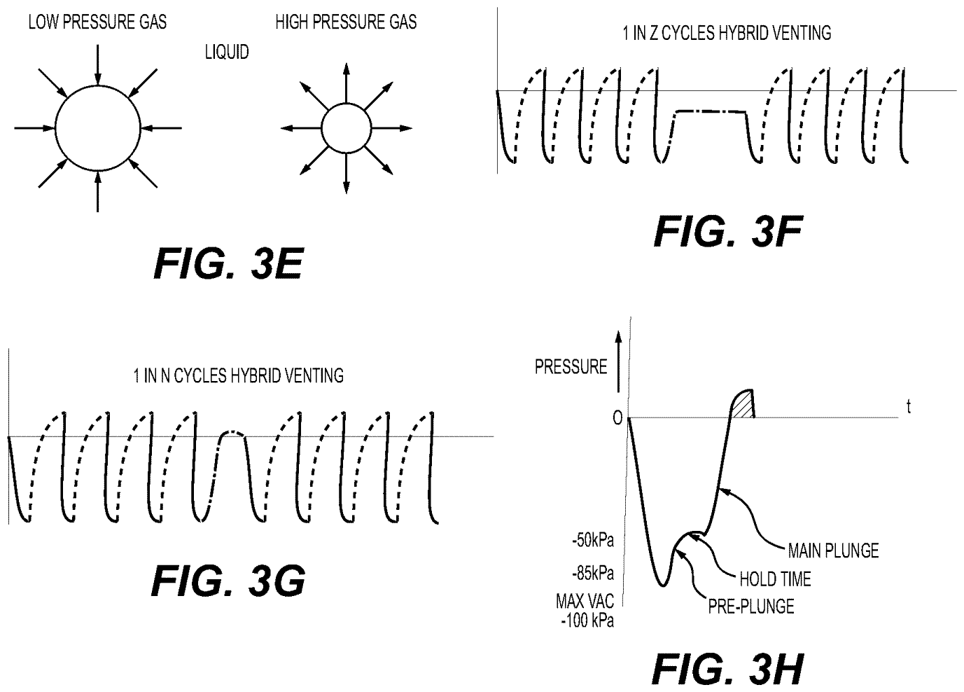

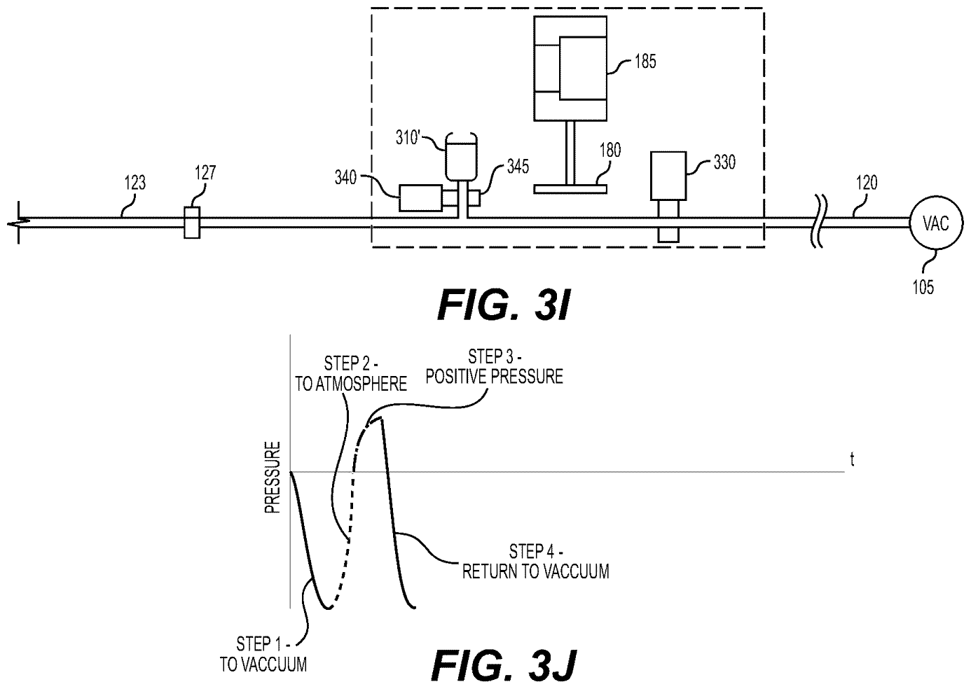

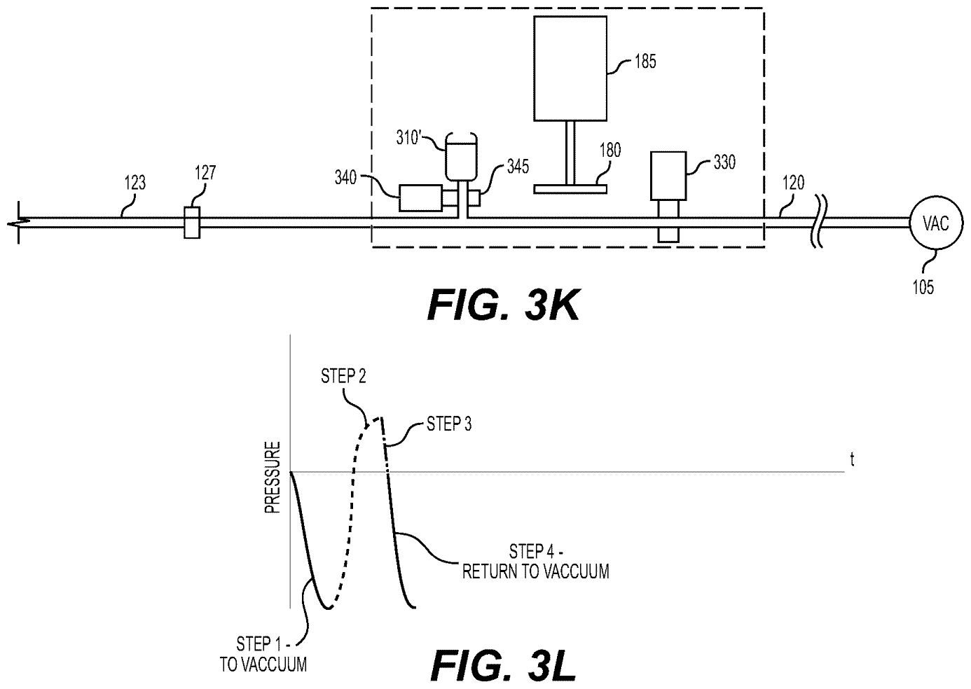

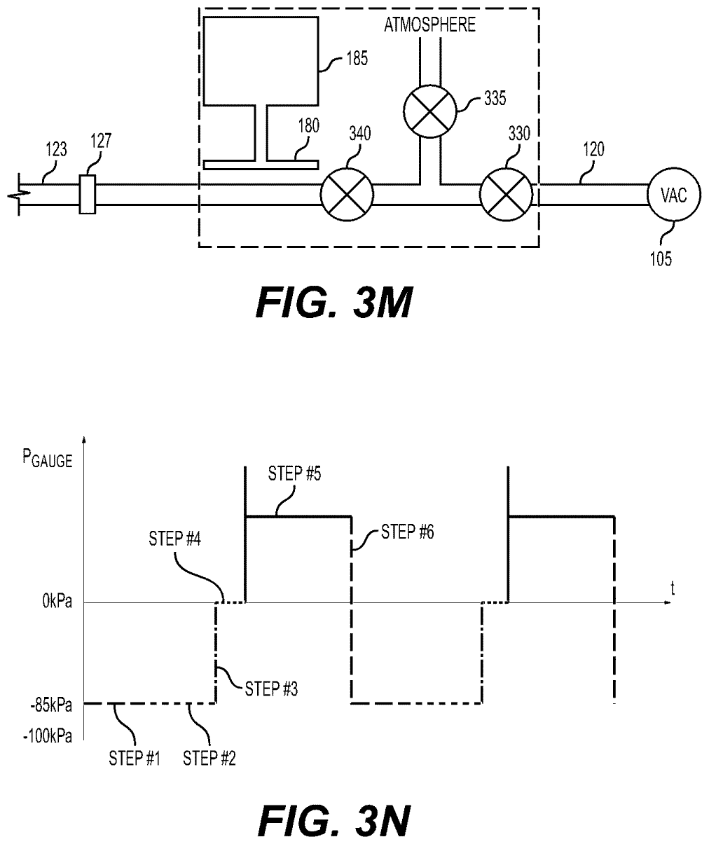

As previously mentioned, the positive pressure inlet tubing 115 of the vented cyclic aspiration system in accordance with the present disclosure may be vented to different positive pressure sources other than the liquid reservoir open to atmospheric pressure 110 illustrated in the example in FIG. 1A to generate the positive pressure pulse. One alternative positive pressure source eliminates the liquid reservoir in FIG. 1A by venting the system only to atmospheric pressure (e.g., FIG. 3J). Still another alternative is to use a closed pressurized reservoir as the positive pressure source. Several illustrative examples of vented cyclic aspiration systems vented to a closed pressurized reservoir having a pressure higher than atmospheric pressure are shown in FIGS. 1B-1D, each of which will now be described. Aside from the number of pressure and/or flow sensors 175, the cyclic aspirations systems in FIGS. 1B-1D are similar to that of FIG. 1A for which the description thereof is provided above. Focus instead will only be in describing the operation by which each closed pressurized reservoir generates the positive pressure pulse in the positive pressure inlet tubing 115. In FIG. 1B the closed pressurized reservoir 110′ is a plunger 190 slidable therein when displaced by an external actuator 185 (e.g., linear actuator, solenoid, cam, reciprocating motor, rotating to reciprocating motor, etc.) thereby generating the positive pressure pulse in the positive pressure inlet tubing 115 as controlled by the second gating device 135. As programmed, controller 165 (e.g., processor) via the external actuator 185 consistently displaces the plunger 190 within the reservoir thereby maintaining the same amplitude of positive pressure pulse generated with each cycle. FIG. 1C illustrates the closed pressurized reservoir as a pressurized prefilled or pre-loaded cartridge 110′ (e.g., a pressurized saline bag). The pressurized prefilled or pre-loaded cartridge may be hung (as depicted in FIG. 1C) or otherwise loaded as a module or cartridge into the system while maintained in a pressurized state (e.g., using a clamp mechanism). In operation, the clamp mechanism is withdrawn whereby the positive pressure enters the system as controlled by the second gating device 135. Yet another example of the closed pressure reservoir is shown in FIG. 1D as an accumulator 110′ such as a flexible bladder/separator (e.g., rubber bladder/separator). Compressed air is present with an upper section of the bladder 110′ (as indicated in FIG. 1D) while against the force of the bladder 110′ liquid is pumped into the lower section of the bladder via a pump 111 and associated third gating device 135′ while in an open state. While the second gating device 135 is closed the pressure within the bladder 110′ builds or accumulates until reaching a desired level or amplitude whereupon the third gating device 135′ is closed. An injection of positive pressure is generated when the pressure accumulated within the bladder 110′ enters the positive pressure inlet tubing 115 as controlled by the second gating device 135.

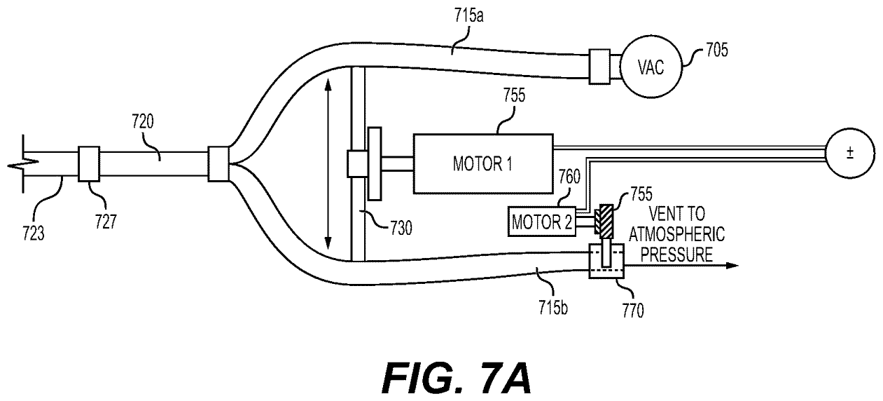

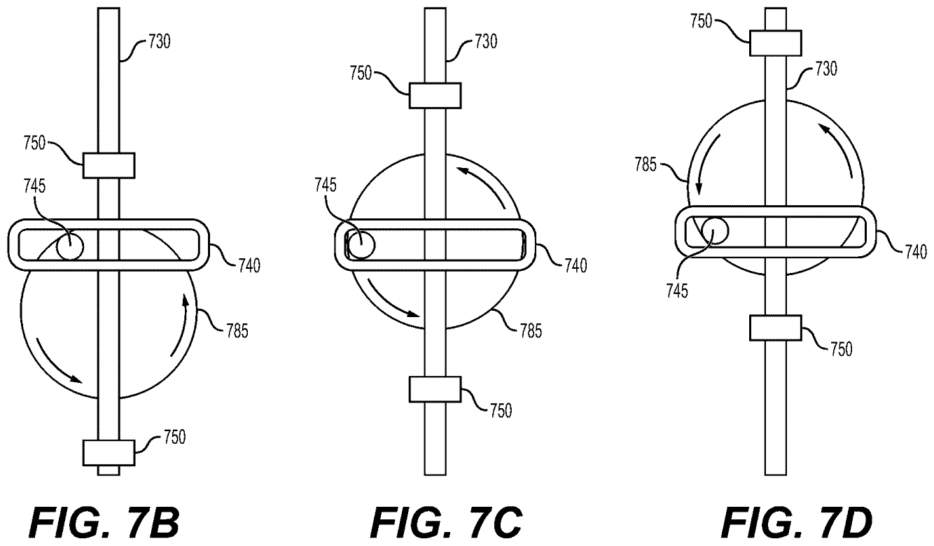

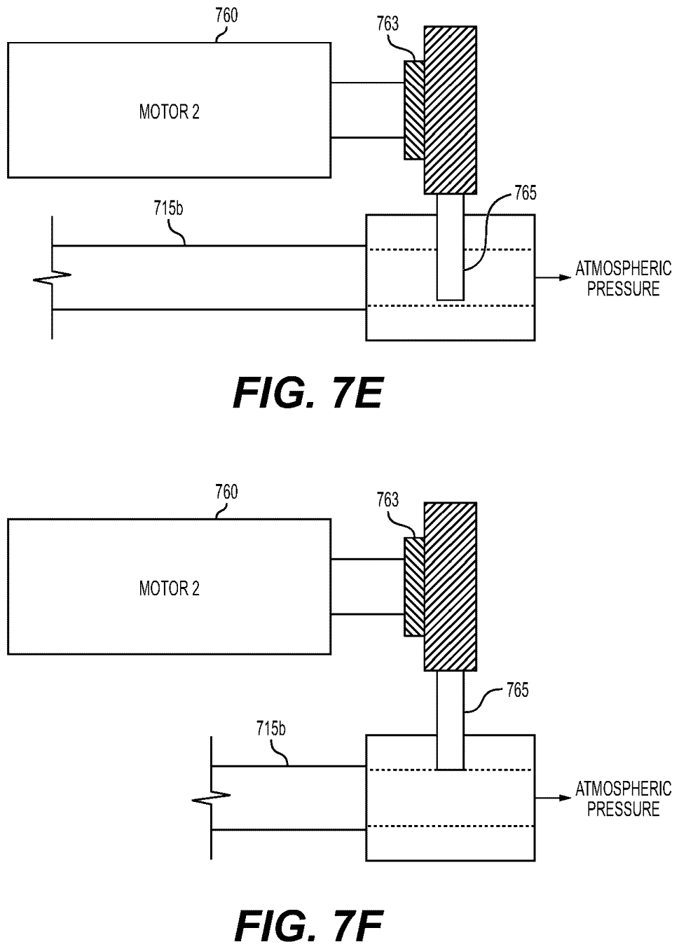

The exemplary vented cyclic aspiration systems in FIGS. 1A-1D employ two gating devices 130, 135 for controlling passage through the vacuum pressure inlet tubing 120 and positive pressure inlet tubing 115, respectively. Alternatively, the vented cyclic aspiration system in FIG. 7A employs a single gating device and rotating-to-linear conversion mechanism (e.g., a scotch yoke mechanism). An aspiration catheter 723 is connected to a proximal hub 727 that, in turn, is connected to inlet tubing 720 the proximal end of which is Y-split into separate vacuum pressure inlet tubing 715a and positive pressure inlet tubing 715b. The vacuum pressure inlet tubing 715a and positive pressure inlet tubing 715b are each made of a deformable, flexible, collapsible, elastomeric material compressible when subject to an external linear force but preferably self-transitioning back to a non-compressed state (original preformed shape) when the external linear force is withdrawn. Disposed between the vacuum pressure inlet tubing 715a and positive pressure inlet tubing 715b is a single external shaft 730 (acting as two gating devices) reciprocally linearly displaceable within securing pins 750 between two maximumly opposing positions (i.e., a first maximumly displaced position and a second maximumly displaced position). Reciprocating linear displacement of the external shaft 730 between the first and second maximum displaced positions may be realized using a rotational-to-linear motion conversion mechanism that includes a rotatable wheel 785 connected to the external shaft 730 via a yoke 740 and a pin 745 following within the yoke 740. Wheel 785 is rotated by a first motor 755 connected thereto via a mounting shaft, wherein the first motor 755 is energized via electrical voltage wires to a power source and preferably allowing variable control of the frequency of rotation of the wheel 785. At any given time, regardless of the position of the rotatable wheel 785 at least one of the vacuum pressure inlet tubing 715a and/or the positive pressure inlet tubing 715b is compressed (i.e., constricting flow therethrough) via one and/or both ends the external shaft 730. FIGS. 7B-7D depict the reciprocating mechanism at different stages or positions of rotation. In FIG. 7B the external shaft 730 is linearly displaced to the first maximum displacement position constricting the vacuum pressure inlet tubing 715a occluding passage of the vacuum pressure generated by the vacuum pump 705 while simultaneously allowing passage of the atmospheric pressure through the positive pressure inlet tubing 715b (not pinched or constricted by the external shaft 730). With continued rotation of the wheel 785 in a counterclockwise direction, in FIG. 7C the external shaft 735 is further displaced linearly to an equilibrium position midway between the first and second maximum displacement positions. The axial length of the external shaft 730 is selected such that when the wheel 785 is rotated to this equilibrium position both the vacuum pressure inlet tubing 715a and the positive pressure inlet tubing 715b are pinched or constricted by respective ends of the external shaft 730 allowing the positive pressure within the system to dissipate due to viscous losses. Still further rotation of the wheel 785 linearly displaces the external shaft 735 to the second maximum displacement position pinching or constricting (e.g., occluding) the positive pressure inlet tubing 715b while simultaneously allowing vacuum pressure to be regenerated in the system via the vacuum pressure inlet tubing 715a (FIG. 7D).

The reciprocating mechanism of FIG. 7A may optionally include a second motor 760 for varying the amplitude of the positive pressure pulse by controlling the size of the opening within the positive pressure pulse inlet tubing 725 vented to atmospheric pressure. FIGS. 7E and 7F depict an enlarged view of a gating device 755 acting as a valve controlling the size of the opening within the pressure pulse inlet tubing 715b using the second motor 760. The second motor 760 controls rotation of a gear 763 which, in turn, is linked to a slotted rotating pin 765 displaceable in/out of a channel defined in the positive pressure inlet tubing 715b adjusting the extent of the opening to atmospheric pressure. Displacement via the second motor 760 of the slotted rotating pin 765 through the channel to a position of maximum extension into the lumen of the positive pressure inlet tubing 715b obstructs (i.e., blocks) passage of the atmospheric pressure therethrough (as depicted in FIG. 7E), while the slotted rotatable pin 765 in a position of maximum retraction fully or completely opens the positive pressure inlet tubing 715b to the atmospheric pressure (as depicted in FIG. 7F). By way of example, the two positions illustrated are that of maximum extension and maximum retraction, respectively, of the slotted rotatable pin 765 representing full closure (i.e., obstructing or blocking) and full opening of the positive pressure inlet tubing 715b to the atmospheric pressure. It is of course possible to adjust the extent of opening within the positive pressure inlet tubing 715b to any desired position between the extremes of maximum extension/advancement (i.e., fully closed, blocking, or obstructing) and maximum retraction (i.e., fully open state). Reducing the extent of the passageway withing the positive pressure inlet tubing 715b reduces the amplitude of the positive pressure pulse generated, whereas increasing the size of the passageway of the positive pressure inlet tubing 715b increases the amplitude of the positive pressure generated therein. The slotted rotatable pin 765 may be positioned anywhere axially along the positive pressure inlet tubing 715b and other gating devices in accordance with the present disclosure may be substituted.

The example reciprocating mechanism in FIG. 7A may be used as a handheld device and preferably positioned as close as possible to the proximal hub 727 to achieve maximum frequency (Hz) for maximum pressure difference, as explained by the following equation:

$$t = \frac{V}{q ( p 0 / p 1 )}$$

where,
   t=evacuation time (seconds)
   V=volume to be evacuated (m3)
   q=pump flow rate (kg/s)
   p0=initial vacuum pressure (absolute pressure)(bar)
   p1=end vacuum pressure (absolute pressure)(bar)

As the volume increases, so does the time to realize, achieve or generate vacuum. Thus, at higher frequencies the amplitude will be dampened or cut off more as the system has less time to realize, achieve, generate, or get up to vacuum pressure, therefore greatly reducing the pressure difference between peak and trough of the cyclic aspiration pressure wave and hence greatly reducing energy per second at the distal tip of the aspiration catheter.

Data processing and input data signals produced using a user interface controlling the reciprocating mechanism may be electrically connected via wires/cables minimizing the overall weight and footprint of the handheld system. It is further contemplated and within the scope of the present disclosure for the handheld device to have associated programming buttons or switches for selecting or toggling among a plurality of available pressure modes (e.g., cyclic aspiration vs. non-cyclic constant aspiration). For instance, following expiration or completion of a preset time for cyclic aspiration, prior to removal of the aspiration catheter from the patient, the mode button may be selected or toggled transitioning to the non-cyclic constant vacuum pressure in order to maximize hold on the captured clot thereby minimizing risk of dislodgement.

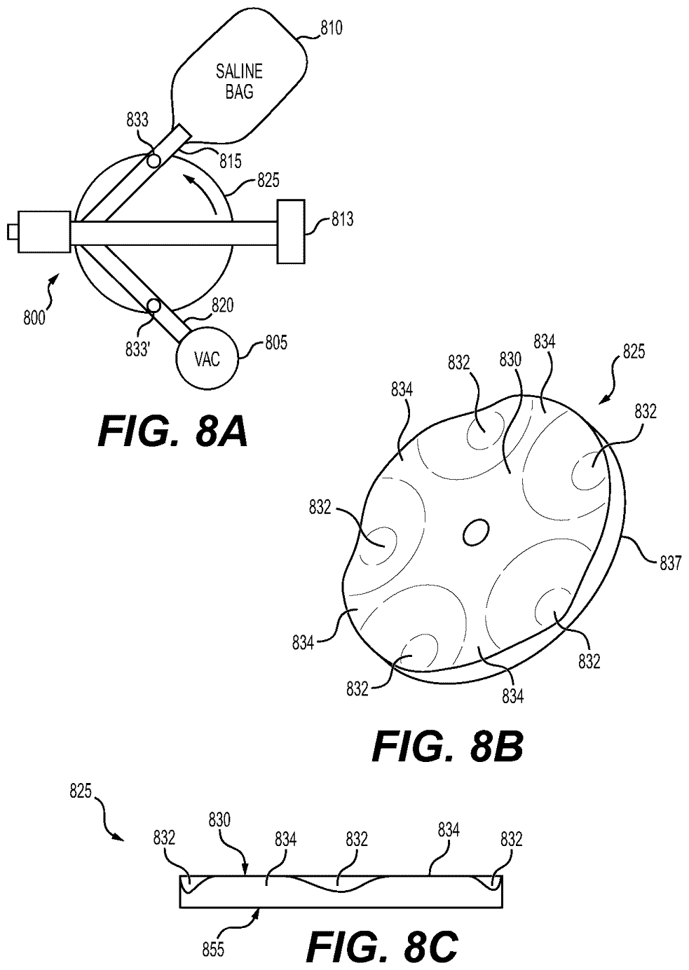

In the vented cyclic aspiration systems described above the positive pressure pulse is produced within inlet tubing connected in fluid communication between the vacuum pump and proximal hub attached to the aspiration catheter. In accordance with yet another aspect of the present disclosure the positive pressure pulse may be generated in a rotating hemostatic valve (RHV) itself. FIG. 8A is a top view of an exemplary rotating hemostatic valve 800 vented to a positive pressure source (e.g., saline bag) adapted to produce a positive pressure pulse. The rotating hemostatic valve shown includes an outlet port and three inlet ports (i.e., a main inlet port 813, a vacuum pressure inlet side port 820 connected to a vacuum pump 805, and a positive pressure inlet side port 815 connected to a liquid reservoir (e.g., saline bag)) 810 as the positive pressure source. Each of the positive pressure inlet side port 815 and the vacuum pressure inlet side port 820 has a gating device (e.g., valve) associated therewith, which in the example of FIG. 8A is a downwardly projecting pin 833, 833′, respectively. Each pin 833, 833′ is upwardly displaceable within a hole defined therein a wall of one of the respective side ports 815, 820. Irrespective of positioning within the hole, at all times the pins 833, 833′ remain externally sealed to the respective inlet side port 815, 820 preventing leakage therethrough the associated hole defined therein. The rotating hemostatic valve has connected thereto a single actuator wheel 825 rotatable (e.g., via a pre-wound or rewindable mechanism) relative thereto intermittently cyclically actuating only one of the pins 833, 833′ at any given time. Actuator wheel 825 is depicted in the top perspective view and side view in FIGS. 8B & 8C, respectively. The actuator wheel 825 has an upper surface 830 facing the rotating hemostatic valve, an opposite lower surface 855 facing away from the rotating hemostatic valve and a circular sidewall 837 extending therebetween. Intermittent cyclic actuation (e.g., upward displacement of only one of the pins 833, 833′ at any given time) using a rotatable single actuator wheel 825 is realized by configuring the upper surface 830 to have a contour representing a 360 degrees radially undulating wave of alternating depression regions 832 and planar regions 834. The pins 833, 833′ associated with the respective inlet side ports 815, 820 following downwards (corresponding to the depression regions 832) or upwards (corresponding to the planar regions 834) the undulating wave contour of the upper surface 830. At any given point of rotation, only one of the pins 833, 833′ is aligned in one of the depression regions 832 while simultaneously the other pin is aligned in one of the planar regions 834. The pin being fully extended downward (i.e., not displaced upwards through the hole) thus opening passage through the respective inlet side port when aligned in one of the depression regions 823, whereas the pin is displaced upwards through the hole thereby obstructing passage therethrough the respective inlet side port when aligned in one of the planar regions 834. Accordingly, with the rotation of a single actuator wheel 825, the pins 833, 833′ act as gating devices (e.g., valves) intermittently cycling between the vacuum pressure and the positive pressure generated from venting of the RHV to the liquid reservoir (e.g., a saline bag). The rotating hemostatic valve itself generates the positive pressure pulse eliminating the need for electronic components reducing the cost of manufacture. Accordingly, all components (i.e., the RHV (including the positive pressure inlet side port 815 and associated pin 833 as well as the vacuum pressure inlet side port 820 and associated pin 833′) and actuator wheel 825) may be discarded after a single use eliminating the risk of clogging.

Next to be described is an example “non-vented” cyclic aspiration system for producing the cyclic aspiration pressure waveform in accordance with the present disclosure wherein the positive pressure pulse is generated by initiating a dual functionality of: (i) controlling passage through the conduit of the vacuum pressure generated by the vacuum pump; and (ii) reducing the internal volume displacing the fluid collected therein thereby generating a positive pressure pulse. These dual functions may be performed using two separate components (e.g., a gating device and a separate positive pressure pulse generator mechanism). Alternatively, the dual functions may be performed by a single integrated positive pressure pulse generator mechanism.

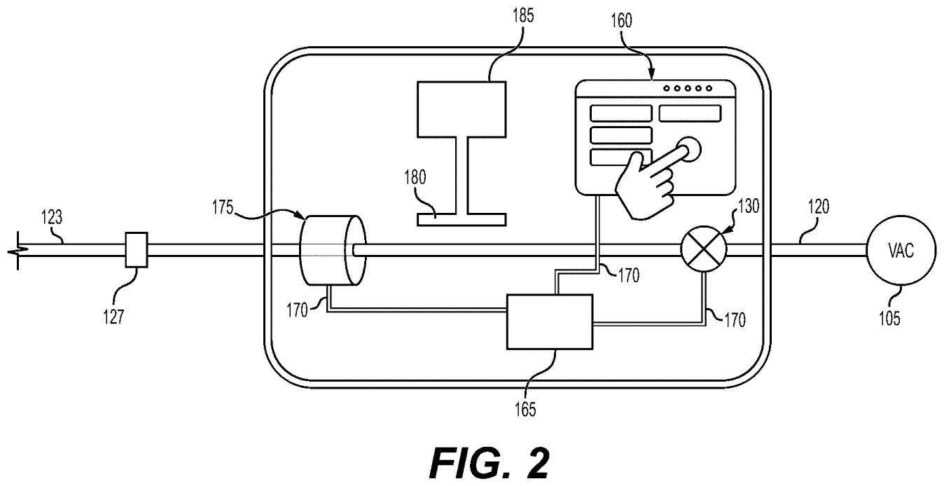

By way of illustrative example, the positive pressure pulse generator mechanism in FIG. 2 is a displaceable plunger 180 applying an external force compressing a section along the vacuum pressure inlet tubing 120 that is flexible, compliant, or compressible in the non-vented cyclic aspiration system. A separate gating device 130 is employed to control passage through the vacuum pressure inlet tubing 120 of the vacuum pressure generated by the vacuum pump 105. Any number of different ways using a wide arrangement of mechanical components may be employed as the positive pressure pulse generator mechanism to apply an external force compressing a section of the flexible inlet tubing to create the positive pressure pulse (i.e., in the inlet tubing). The underlying principle for generating the positive pressure pulse in the non-vented cyclic aspiration system being that externally compressing a section of the flexible inlet tubing reduces its internal volume displacing existing fluid therein thereby generating the positive pressure pulse (i.e., injection of positive pressure). External compression of the flexible vacuum pressure inlet tubing to produce the positive pressure pulse may be accomplished using, for example, a displaceable plunger 180 (FIG. 2), a compressible bladder, a rotatable arm, a pair of electromagnets, or a compression plate. The positive pressure pulse generator mechanism is transitionable between a state in which it applies an external force compressing the section of flexible vacuum pressure inlet tubing and a state in which the external compressive force is withdrawn from the vacuum pressure inlet tubing using an actuator (e.g., a linear actuator, solenoid, cam, reciprocating motor, rotating reciprocating motor, etc.) arranged externally of the flexible vacuum pressure inlet tubing. Recovery or restoration time representing the time it takes for the compressed flexible vacuum pressure inlet tubing to return to a non-compressed state upon withdraw of the external force is a key concern in attaining a sufficiently rapid, maximum, or high cycling frequency (e.g., approximately 1 Hz to approximately 20 Hz). Insufficiently slow recovery or restoration time is attained by simply allowing the compressed flexible vacuum pressure inlet tubing to naturally (i.e., unforced, unassisted, or unaided) return to its non-compressed state upon withdraw of the external compressive force by the actuator. Therefore, to maximize cycling frequency (i.e., minimize restoration and recovery time) return of the compressed flexible vacuum pressure inlet tubing to its non-compressed state upon withdraw of the external force is forced, assisted, or aided in some manner in accordance with the present disclosure. Assistance in hastening return of the compressed flexible vacuum pressure inlet tubing to its non-compressed state may be provided by an internal restoring or recovery member disposed in the flexible inlet tubing coinciding with the section undergoing compression. For instance, to hasten, force or assist return to its non-compressed state or shape the flexible vacuum pressure inlet tubing may have disposed therein a restorative radially self-expandable braid, cage, skeleton, or other shape memory material member returnable to its original shape upon withdraw of the applied external force via the actuator. It is also contemplated that such assistance may be provided by holding or maintain in the place the compressed flexible vacuum pressure inlet tubing while being subjected to externally applied forces (e.g., electromagnetic or mechanical) in tandem in opposing directions (i.e., externally pulling apart the compressed flexible vacuum pressure inlet tubing). Return of the compressed flexible vacuum pressure inlet tubing to its non-compressed state may be further hastened or assisted by employing extruded flexible vacuum pressure inlet tubing having a non-circular radial cross-section (e.g., lips along parallel sides in an axial direction) exhibiting an axial resistance overcome when subject to the compressive external force via the actuator.

In an alternative example the positive pressure pulse generator mechanism may be a housing having at least one displaceable component slidable therein to perform two actions: as a gating device for controlling passage therethrough of the vacuum pressure, while also generating the positive pressure pulse. By way of non-limiting examples, the single integrated displaceable component may be at least one piston or plunger slidable within the housing via an actuator (e.g., a plurality of electromagnets, linear actuator, solenoid, cam, reciprocating motor, rotating reciprocating motor, etc.) arranged externally of the housing. In the case of more than one displaceable member displaceable in the housing, each performs one of the two actions in response to a single actuator or multiple actuators operating independently of each other. For example, the more than one displaceable member may be pistons or plungers slidable within the housing, or an internally projecting ball valve secured to a flexible diaphragm/membrane stretched across an opening of the housing.

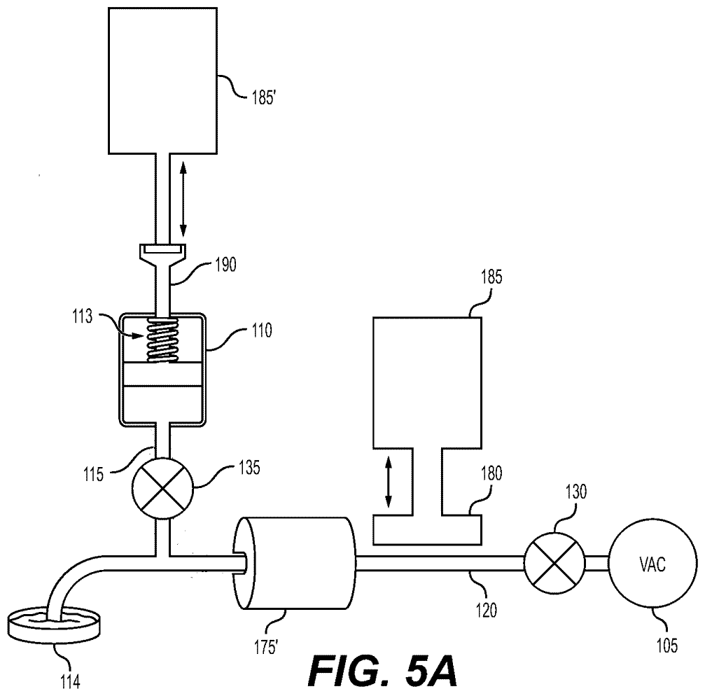

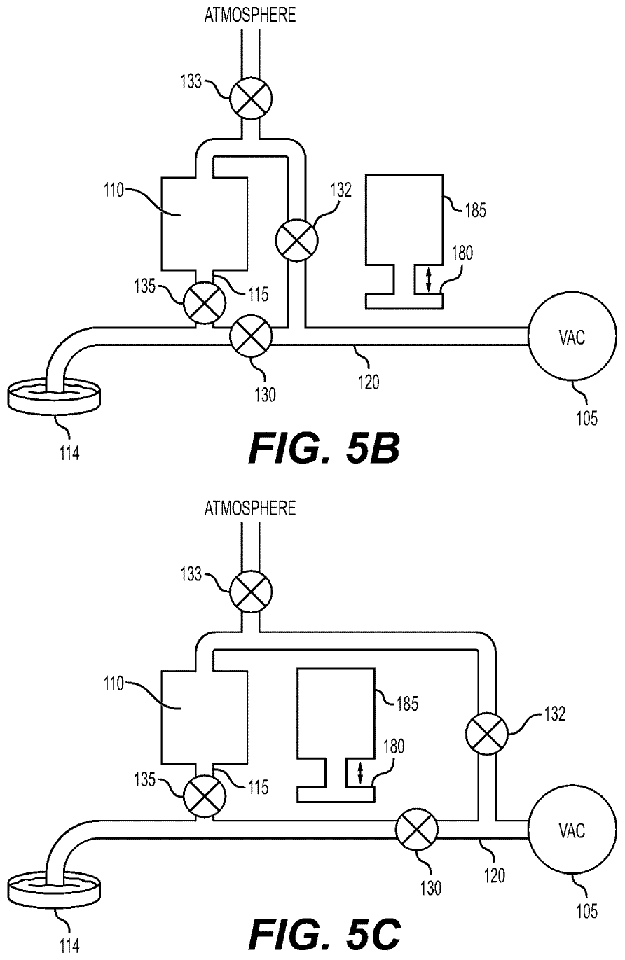

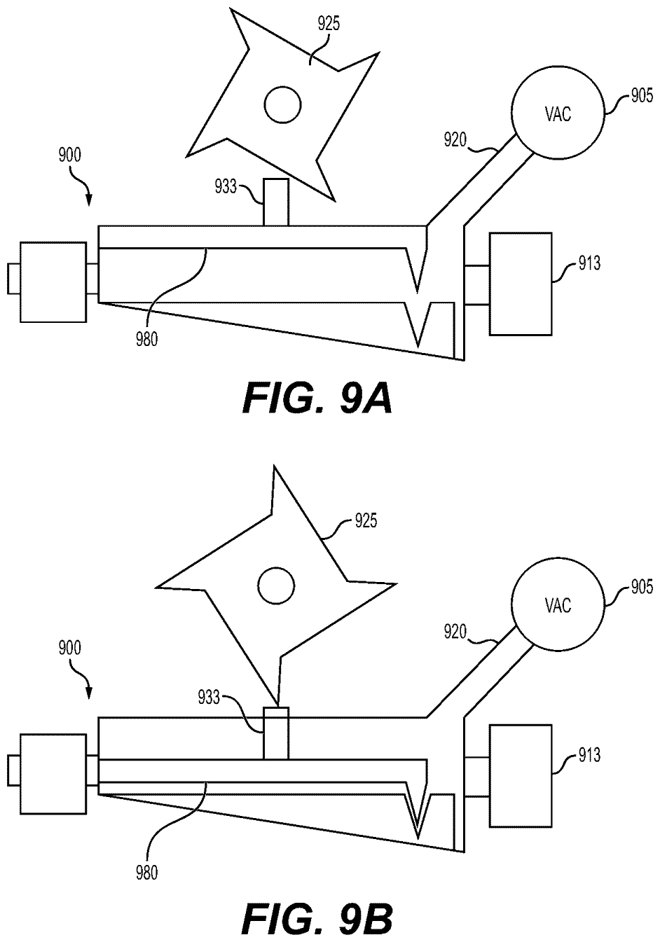

In the example non-vented cyclic aspiration system in FIG. 2 the positive pressure pulse is produced within the vacuum pressure inlet tubing connected in fluid communication between the vacuum pump and proximal hub attached to the aspiration catheter. In accordance with yet another aspect of the present disclosure the positive pressure pulse may be generated in a non-vented cyclic aspiration system within the rotating hemostatic valve (RHV) itself. FIGS. 9A & 9B illustrate another example of a rotating hemostatic valve 900 (non-vented, not vented to a positive pressure source such as a saline bag) having two inlet ports (i.e., a main inlet port 913 and a vacuum pressure inlet port 920 connected in fluid communication to a vacuum pump 905). Disposed within the rotating hemostatic valve is an internal displaceable member 935 (e.g., a plunger) to which is attached a gating element 933 (e.g., pin acting as a valve) projecting upwards through a hole, opening, or channel defined in the wall of the RHV and sealed externally thereof to prevent leakage therethrough. An actuator wheel 925 is rotatable (e.g., via a pre-wound or rewindable mechanism) to intermittently cyclically engage/disengage with the gating element 933 (e.g., pin) displaceable through the hole, opening, or channel defined in the wall of the RHV. With the rotation of the actuator wheel 925, intermittently cyclically displacing (i.e., advancing) the gating element 933 (e.g., pin) internally through the hole, opening, or channel. When not displaced by the actuator wheel 925, the gating element 933 (e.g., pin) is maintained in a default retracted position fully raised externally through the hole, opening, or channel allowing unobstructed passage therethrough the RHV of the vacuum pressure generated by the vacuum pump 905 (FIG. 5A wherein the vacuum pressure passes beneath the raised/retracted plunger 980. During continued rotation, the actuator wheel 925 engages with thereby displacing (i.e., advancing) together the pin 933 and plunger 980 secured thereto (FIG. 9B). Advancement of the pin 933 through the hole, opening, or channel defined in the RHV acts as a valve blocking or occluding passage of the vacuum pressure generated by the vacuum pump 905. Simultaneously therewith advancement of the plunger 980 reduces the internal volume in the RHV displacing the fluid collected therein thereby generating a positive pressure pulse (i.e., injection of positive pressure). The rotating hemostatic valve itself generates the positive pressure pulse eliminating the need for electronic components reducing the cost of manufacture. Accordingly, all components (i.e., the RHV (including the main inlet port 913, the vacuum pressure inlet side port 920, plunger 980 and associated pin 933)) may be discarded after a single use thereby minimizing the risk of clogging.

The last cyclic aspiration system in accordance with the present disclosure, herein referred to as a hybrid cyclic aspiration system, combines aspects or features of the vented cyclic aspiration system and the non-vented cyclic aspiration system into a “hybrid” thereof. Specifically, the hybrid cyclic aspiration system incorporates the pressure pulse generator mechanism producing the positive pressure pulse by reducing the internal volume displacing the fluid collected therein thereby generating a positive pressure pulse (i.e., injection of positive pressure) or using a pressurized closed reservoir. The hybrid cyclic aspiration system is also vented to a positive pressure source for the purpose of preventing or minimizing decay/dampening over time of the positive pressure pulse produced, as elaborated in further detail below. As mentioned previously the positive pressure source includes: (i) atmospheric pressure; (ii) a liquid reservoir open to atmospheric pressure and filed with a liquid (e.g., blood and/or saline); or (iii) a pressurized closed reservoir higher than atmospheric pressure.

FIG. 3A is an example of the hybrid cyclic aspiration system in accordance with the present disclosure. While a first gating device 130 controls passage therethrough of the vacuum pressure generated by the vacuum pump 105, a positive pulse generator mechanism (e.g., external plunger 180) externally compresses a section along the flexible vacuum pressure inlet tubing 120, reducing the internal volume displacing the fluid collected therein thereby generating the positive pressure pulse. Although the plunger 180 is shown in FIG. 3A, the positive pressure pulse may be generated using any of the previously described examples, e.g., other mechanisms for externally compressing the flexible inlet tubing, at least one displaceable member within a housing, or the pressurized closed reservoir having a pressure higher than atmospheric pressure. In addition, in FIG. 3A the positive pressure generator mechanism (e.g., plunger 180) is shown positioned on the distal side of the liquid reservoir open to atmospheric pressure 110 but may be positioned on either the proximal or distal side. Positive pressure inlet tubing 115 vented to a liquid reservoir open to atmospheric pressure and filled with a liquid (e.g., saline and/or blood) 110 includes a second gating device 135 controlling intake of atmospheric pressure to prevent or reduce dampening or decay of the positive pressure wave over time. This hybrid cyclic aspiration system optimizes continuity of the pressure waveform produced at the distal tip of the aspiration catheter while also allowing for active control via a controller 165 (e.g., processor) of the amplitude of the vacuum pressure, amplitude of the positive pressure pulse and/or cycling frequency in response to data received from pressure and/or flow sensor(s) 175 along with input from a user interface 160. The principles behind the venting of the hybrid cyclic aspiration system to a positive pressure source to reduce or prevent dampening or decay of the positive pressure wave over time are explained in detail below.

Saline (i.e., mix of water and salt) and blood are liquids used in the cyclic aspiration system. Both liquids contain nitrogen which is dissolved in fluid. Henry's law is a gas law that states that the amount of dissolved gas in a liquid is directly proportional to its partial pressure above the liquid. When the vacuum pump is turned on causing the pressure in the cyclic aspiration system to drop, the liquid collected therein begins to evaporate causing bubbles to form. When subject to vacuum pressure, water in the system is oversaturated with air (e.g., at least approximately 666%). With the decrease in pressure, bubbles escape from the fluid and are more significant than under atmospheric pressure. FIG. 3B illustrates heterogenous bubble nucleation depicting different stages of bubble growth or formation. Specifically, on the left depicts an oversaturated (e.g., supersaturated) gas molecule or packet starting to form on a solid support, the middle depicts a formed bubble, while the right depicts detachment of the bubble. During cycling, the fluid in the system is subjected to cycling between vacuum pressure (i.e., low or minimum pressure) and positive pressure (i.e., high or peak pressure). At a slow rate of change in low (e.g., vacuum) and higher pressures, bubbles should grow under vacuum pressure and then recede at the higher pressure, however, during cycling, more gas tends to enter the bubble during bubble expansion when the surface area is larger, than out during bubble compression when surface area is smaller. Over time this results in a net gain in bubble growth during cycling effecting acoustic properties of the water and/or blood (FIG. 3C illustrating a relatively large bubble impeding the wave causing dampening). Altered velocity causes reflection and refraction to occur at the water-bubble-interface, while the energy extracted from a second wave, by the damped, pulsating bubbles attenuates the wave. The acoustic characteristics depend on the physical properties of the mixture, bubble size and volume ratio of air to water, and the imposed frequency. In summary, bubbles trapped in the inlet tubing of the cyclic aspiration system grow and amalgamate causing compressibility while reducing wave velocity. The degree to which attenuation will occur depends on bubble size, volume ratio of air to water, and imposed pressure wave frequency.

Bubble growth rate may be reduced in several different ways. One way to reduce the rate of bubble growth is by using inlet tubing connecting in fluid communication the vacuum pump to the aspiration catheter having a larger inner diameter. The larger the inner diameter increases the ratio of internal liquid volume to inner tube surface area. In addition, less friction and more liquid volume produces less internal tube restrictions. Another way of reducing bubble growth is to line or coat the inner wall of the inlet tubing with a hydrophilic coating (e.g., highly lubricious material such as PTFE). Still yet another way of reducing bubble growth is by intentionally mechanically bursting the bubbles or diverting their path to a section of tubing outside the cycling path.

The hybrid cyclic aspiration systems in accordance with the present disclosure mitigate the attenuation experienced in the non-vented cyclic aspiration systems over time. These hybrid cyclic aspiration systems are an adjunct of the non-vented system with active cycling (e.g., advancement and retraction of the plunger) to produce the positive pressure interval of the cyclic aspiration pressure waveform. Reducing the bubble growth lessens the extent to which the smaller size bubble blocks or disrupts the fluid path thereby reducing or lessening dampening effect (FIG. 3D in comparison to the larger size bubble in FIG. 3C). The hybrid cyclic aspiration system reduces the rate of bubble growth by changing the pressure of the fluid existing in the system with reduced energy input compared to non-vented cyclic aspiration system, as shown in FIG. 3E. That is, the pressure in the hybrid cyclic aspiration system is changed from full vacuum pressure to atmospheric pressure without active plunging which requires higher energy. It is also proposed that as bubbles grow in the hybrid cyclic aspiration system, the volume of the liquid reduces in the inlet tubing and the possibility of tube expansion. During each cycle, the liquid reservoir in the hybrid cyclic aspiration system replenishes the lost volume of fluid and prevent bubbles from blocking the cycling path. Also, a lower positive pressure pulse (e.g., a representative example of which is shown in FIG. 3F) or simply a short period of vacuum constant or non-constant (e.g., a representative example of constant vacuum is shown in FIG. 3G) may be used intermittently to eliminate or minimize bubbles in the system. While yet another available option, is to employ a two-stage pressure plunge (e.g., a representative example of which is shown in FIG. 3H). The two-stage pressure plunge includes a pre-plunge raising the pressure from “full vacuum pressure” to an intermediate level, preferably approximately −50 kPa, but at least ≥approximately −60 kPa. Followed thereafter by a predetermined hold/dwell time. Preferably, the dwell time is less than approximately 30% of the overall pulse cycle time. By way of example, for a 1 Hz frequency the dwell will be less than approximately 0.3 secs, whereas for a 20 Hz frequency the dwell time will be less than approximately 0.015 secs. After the passage of the dwell time, the main plunge occurs. This two-stage plunge reduces the amount of energy imparted to the fluid in comparison to that of a single plunge, thereby minimizing bubble growth.

FIG. 3I is another example hybrid cyclic aspiration system that once again minimizes bubble growth rate and allows for wave continuity. The positive pressure pulse generator mechanism 180 (e.g., displaceable plunger externally compressing a section of flexible vacuum pressure inlet tubing 120) is disposed between a first gating device 340 (e.g., pinch valve) and a second gating device 330 (e.g., pinch valve). While at maximum “full” vacuum, the system is opened to atmospheric pressure until the fluid pressure therein reaches approximately atmospheric pressure before the plunger 180 is advanced via an externally arranged actuator 185 (e.g., linear actuator, solenoid, reciprocating motor, cam, rotating reciprocating motor, etc.) injecting additional controlled positive pressure into the system. The time it takes for the fluid pressure in the system when opened to the atmosphere to reach approximately atmospheric pressure (e.g., approximately 10% of the cycle time) changes with frequency. Once the fluid pressure in the system has reached atmospheric pressure the plunger 180 is advanced injecting further controlled positive pressure into the system. This minimizes bubble growth rate and allows for wave continuity. This is achieved by connecting a T-connector 345 to inlet tubing 120 (e.g., main vacuum line) pinched shut by the first gating device 340 (e.g., pinch valve) until required. When required, the first gating device 340 (e.g., pinch valve) opens allowing a fluid connection to atmosphere via a fluctuation reservoir 310 open to the atmosphere. This system requires at most an approximate 60 ml volume fluctuation reservoir 310 as the fluid contained therein is not used as an energy source for creating the positive pressure interval of the cyclic aspiration pressure waveform, but merely as a fluctuation vessel to achieve atmospheric pressure. Additional advantages provided by this alternative hybrid system is that it prevents wave damping and allows for cycling at maximum “full” vacuum. It should be noted that the fluctuation reservoir 310 may be opened each cycle or every “N” number of cycles if deemed more suitable (FIG. 3F). It may be beneficial to open the second gating device 330 (e.g., pinch valve) while the first gating device 340 (e.g., pinch valve) is open every “Z” cycles to allow fluid flow from the fluctuation reservoir 310 to the vacuum pump 105 to replenish part or most volume around the active cycling device area (FIG. 3G). This will advantageously eject fluid along with bubbles into the vacuum pump while also allowing fresh fluid to be cycled once again. Referring to the exemplary representative pressure waveform depicted in FIG. 3J, in operation initially to generate the vacuum pressure interval of the cyclic aspiration waveform, the first gating device 340 (e.g., pinch valve) is closed, while the second gating device 330 (e.g., pinch valve) is open and the plunger 180 is retracted so that the vacuum pressure inlet tubing 120 drops to maximum “full” vacuum (e.g., approximately −85 kPa) (“step 1” in FIG. 3J). Next, the first gating device 340 (e.g., pinch valve) is opened simultaneously as the second gating device 330 (e.g., pinch valve) is closed (“step 2” in FIG. 3J). This allows the vacuum pressure inlet tubing 120 to vent to the fluctuation reservoir 310 raising the pressure towards atmospheric pressure thereby allowing any bubbles that form to escape. While the second gating device 330 (e.g., pinch valve) is closed, the first gating device 340 (e.g., pinch valve) is opened and the plunger 180 is advanced (i.e., extended) injecting additional positive pressure into the system (“step 3” in FIG. 3J). Lastly, the second gating device 340 (e.g., pinch valve) is opened while the plunger 180 is retracted sending or cycling the system back to maximum “full” vacuum pressure (“step 4” in FIG. 3J). Advantageously, this system in FIG. 3I only requires a relatively small volume (e.g., approximately 60 ml) fluctuation reservoir 310, eliminating the need for a saline bag or larger volume reservoir.

Another alternative arrangement to the hybrid cyclic aspiration system is shown in FIG. 3K. This hybrid cyclic aspiration system operates in the same manner but allows for atmospheric venting post advancement (e.g., plunging) of the plunger 180 to inject the positive pressure into the system. The pressurized fluid exhausts through the fluctuation reservoir 310. Referring to the pressure waveform depicted in FIG. 3K, in operation initially to generate the vacuum pressure interval of the cyclic aspiration waveform, the first gating device 340 (e.g., pinch valve) is closed, while the second gating device 330 (e.g., pinch valve) is open and the plunger 180 is retracted so that the vacuum pressure inlet tubing 120 drops to maximum “full” vacuum (e.g., approximately −85 kPa) (“step 1” in FIG. 3L). Next, the plunger 180 is advanced (e.g., extended) positively pressurizing the system (“step 2” in FIG. 3L). Thereafter, the first gating device 340 (e.g., pinch valve) is opened simultaneously as the second gating device 330 (e.g., pinch valve) is closed (“step 3” in FIG. 3L). Lastly, the second gating device 340 (e.g., pinch valve) is opened while the plunger 180 is retracted reducing the pressure in the system cycling back to maximum “full” vacuum pressure (“step 4” in FIG. 3L). Once again, this system in FIG. 3K only requires a relatively small volume (e.g., approximately 60 ml) fluctuation reservoir 310, eliminating the need for a saline bag or larger volume reservoir.

While still another modification of the hybrid cyclic aspiration system is provided in FIG. 3M that utilizes the water “hammer effect” to prevent deterioration of the cyclic wave. The pinch valves 340, 335, 330 open and close per the legend accompanying the example graphical representation of the pressure waveform in FIG. 3N which allows air to rush in and hammer the water column which is contained in the system from the device to the distal tip/end of the aspiration catheter. In contrast to the hybrid cyclic aspirations systems in FIGS. 3I & 3K, the system in FIG. 3M eliminates the need for a fluctuation reservoir as air from the atmosphere rather than saline is taken into the positive pressure inlet tubing 115 proximally of the plunger 180. Since the system is open to atmospheric pressure, during each cycle, bubble growth is reduced, as described above. In this system, the air and water column do not mix. The air hammer impacts (hits off the water column) around the location of the first gating device 340 (e.g., pinch valve) allowing the system to head towards atmospheric pressure before the plunger 180 injects additional positive pressure into the system.

Referring to the example graphical representation in FIG. 3N, initially the first and third gating device 340, 330 (e.g., pinch valves) are open, while the second gating device 335 (e.g., pinch valve) is closed, allowing maximum “full” vacuum pressure (“step 1” in FIG. 3N). Next, the third gating device 330 (e.g., pinch valve) is closed (“step 2” in FIG. 3N). The second gating device 335 (e.g., pinch valve) is then opened allowing the pressure in the system to reach approximately atmospheric pressure (“step 3” in FIG. 3N). Briefly the second gating device 335 (e.g., pinch valve) is closed for a dwell time (“step 4” in FIG. 3N). Preferably, the dwell time is less than approximately 30% of the overall pulse cycle time. By way of example, for a 1 Hz frequency the dwell will be less than approximately 0.3 secs, whereas for a 20 Hz frequency the dwell time will be less than approximately 0.015 secs. Thereafter, the plunger 180 is advanced injecting positive pressure into the system (“step 5” in FIG. 3N) that dissipates in the system over time. Lastly, the plunger 180 is retracted while simultaneously the first and third gating device 340, 330 (e.g., pinch valves) are opened restoring the system to maximum “full” vacuum pressure.

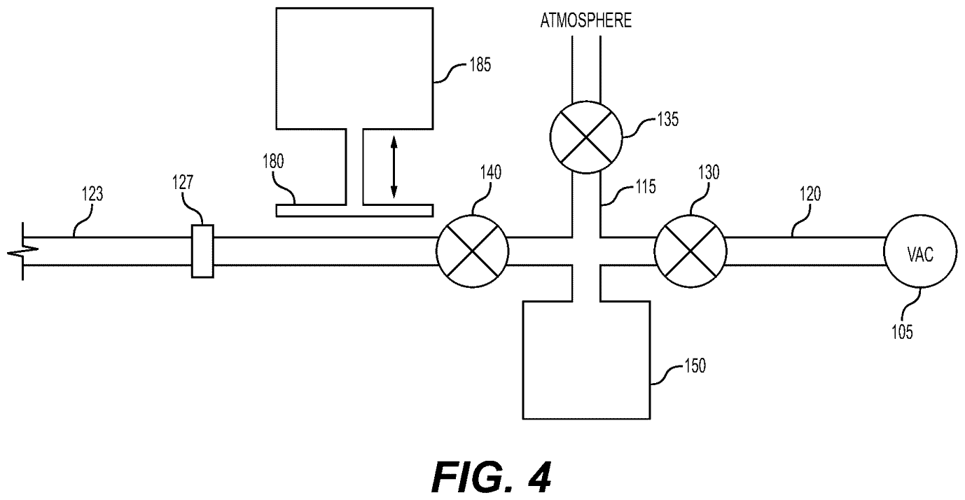

The hybrid cyclic aspiration system of FIG. 3M may be modified to include an optional surge or buffer tank 150 disposed between the first and third gating device 340, 330 (e.g., pinch valves) to optimize hammer spikes, as shown in FIG. 4. Surge tank 150 (e.g., a vacuum pressure syringe) mitigates the impact of the positive pressure pulse (i.e., positive pressure surge or injection) in the positive pressure inlet tubing 115 when the second gating device 135 is opened. A supplemental gating device 140 isolates the positive pressure pulse generator mechanism 180 (e.g., displaceable plunger) from the surge tank 150, otherwise the surge tank 150 would nullity the stroke of the positive displacement pressure mechanism 180.

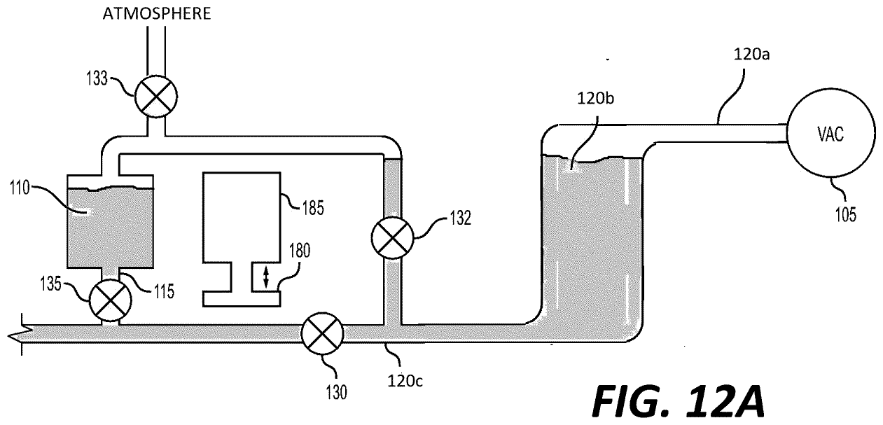

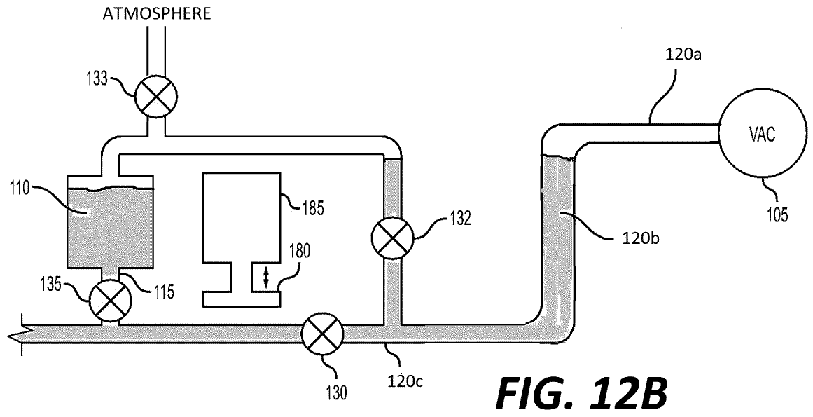

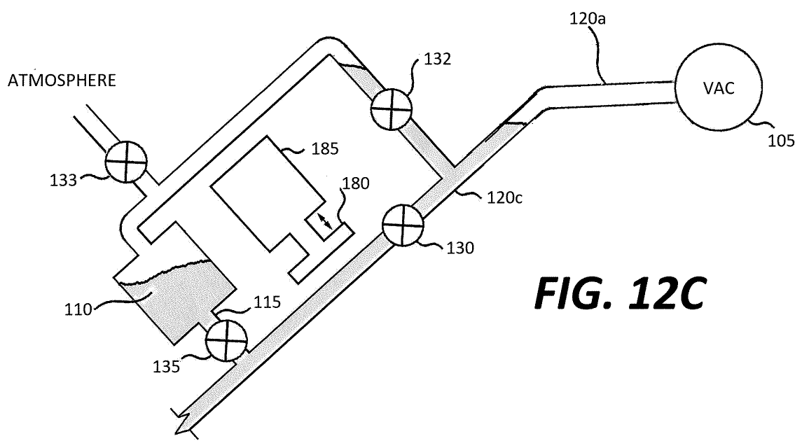

Any voids or gas in the vacuum pressure inlet tubing at the positive pressure pulse generator mechanism would have a negative effect on the amplitude and propagation of the positive pressure wave. Maintaining fluid in the vacuum pressure inlet tubing at the positive pressure pump generator mechanism ensures effective creation and propagation of the positive pressure wave through the vacuum pressure inlet tubing to the aspiration catheter and clot. To ensure that fluid is maintained in the vacuum pressure inlet tubing at the positive pressure pulse generator mechanism the vacuum pressure inlet tubing at the vacuum pump is preferably arranged higher relative to the vacuum pressure inlet tubing at the positive pressure pulse generator mechanism. Several non-limiting examples of how this may be achieved is illustrated in the hybrid cyclic aspiration systems in FIGS. 12A-12C.

FIG. 12A diagrammatically depicts an example hybrid cyclic aspiration system in accordance with the present disclosure wherein the vacuum pressure inlet tubing 120a at the vacuum pump 105 is higher relative to the vacuum pressure inlet tubing 120c at the positive pressure pulse generator mechanism (e.g., plunger 180) with a vertical offset section of the vacuum pressure inlet tubing 120b therebetween forming an effective reservoir (e.g., mini reservoir). The effective reservoir (e.g., mini reservoir) ensures the presence of fluid in the vacuum pressure inlet tubing 120c at the positive pressure pulse generator mechanism 180 by preventing all the fluid from draining out at the vacuum pump 105. FIG. 12B diagrammatically depicts a modification of the example hybrid cyclic aspiration system of FIG. 12A illustrating that the effective reservoir 120b need not be that large. In the example of FIG. 12B the effective reservoir 120b (e.g., petite reservoir) is smaller in volume than that of the mini reservoir in FIG. 12A. While yet another example is depicted in FIG. 12C in which the entire hybrid cyclic aspiration system (including the vacuum pressure inlet tubing 120c at the positive pressure pulse generator mechanism) is inclined either vertically/perpendicularly or at an angle relative to the vacuum pressure inlet tubing 120a at the vacuum pump 105. The two gating devices 132, 133 may be combined into a single gating device in FIGS. 12A-12C. Furthermore, by way of illustration the positive pressure pulse generator mechanism depicted in FIGS. 12A-12C is a liquid reservoir open to atmospheric pressure 110, but may otherwise be a pressurized closed reservoir 110′ (e.g., the pressurized closed reservoir 110′ with plunger 190 in FIG. 1B).

As mentioned above, the venting of the hybrid cyclic aspiration system to a liquid reservoir open to atmospheric pressure filed with a liquid (e.g., blood and/or saline)) advantageously prevents or minimizes decay/dampening over time of the positive pressure pulse.

Prepping prior to use by flushing the system with saline ensures that existing gas in the system that is highly compressible is purged and replaced with liquid which in contrast is substantially, almost completely, incompressible. The consequence of having gas bubbles in the system is that when the positive pressure pulse is generated in accordance with any of the examples described herein (e.g., external compression of the inlet tubing, at least one displaceable member within a housing, or via a pressurized closed reservoir at a pressure higher than atmospheric pressure) the positive pressure pulse generated compress and reduces the volume of the gas bubbles. Thus, the fluid displacement and thus the positive pressure at the catheter distal tip is reduced negatively impacting clot ingestion. However, “prepping” or flushing of the hybrid cyclic aspiration system during pre-treatment (prior to use) can be challenging for the physician or interventionalist in that the liquid reservoir must be filled with a liquid. To address this concern, the present disclosure contemplates utilizing the vacuum pump to fill the liquid reservoir. Accordingly, the actions on behalf of the physician or interventionalist are simplified down to a single step of merely dipping, placing, or positioning the distal tip of the vacuum pressure inlet tubing 120 into a container (e.g., dish) containing saline allowing the vacuum pump to perform the task of filling the liquid reservoir. Several non-limiting illustrative examples of this automatized or self-prepping system utilizing the vacuum pump to fill the liquid reservoir are shown in FIGS. 5A-5C. It is noted that the positive pressure pulse generator mechanism (e.g., displaceable plunger 180 and associated actuator 185 (e.g., linear actuator, solenoid, cam, reciprocating motor, or rotating to reciprocating motor, etc.)) is depicted in FIGS. 5A-5C positioned on a proximal side of the liquid reservoir 110, but it is also contemplated for it to be positioned on a distal side of the liquid reservoir 110 (as shown in FIG. 3A).

In a first example automatized or self-prepping system in FIG. 5A, initially the distal tip/end of the vacuum pressure inlet tubing 120 is dipped, placed, or positioned within a dish 114 containing saline. While the second gating device 135 is closed and the first gating device 130 is open, the vacuum pump 105 is turned on purging the vacuum pressure inlet tubing 120 until a flow sensor 175′ confirms the vacuum pressure inlet tubing 120 is filled with the saline from the dish 114. Then, the first gating device 130 is closed while opening the second gating device 135 associated with the positive pressure inlet tubing 115 vented to the liquid reservoir 110. A piston 190 is disposed within the liquid reservoir 110 is connected to an actuator 185′ (e.g., linear actuator or solenoid). By retracting the piston 190, saline in the vacuum pressure inlet tubing 120 is drawn into the positive pressure inlet tubing 115 filling the liquid reservoir 110. Once filled, the piston 190 may be disconnected from the actuator 185′. Piston 190 is preferably spring-loaded 113 to maintain a desired pressure level in the liquid reservoir 110 and/or counteract any friction between the piston 190 and inner wall of the liquid reservoir 110.

Another example automatized or self-prepping system is shown in FIG. 5B. Initially the distal tip/end of the vacuum pressure inlet tubing 120 is dipped, placed, or positioned within a dish 114 containing saline. While the second and third gating devices 135, 132 are closed and the first gating device 130 is open, the vacuum pump 105 is turned on purging vacuum pressure the inlet tubing 120 until a flow sensor 175′ confirms the vacuum pressure inlet tubing 120 is filled with the saline from the dish 114. Then, with the first and fourth gating devices 130, 133 closed opening the second and third gating device 135, 132 allowing a vacuum created to purge the liquid reservoir 110 while filling with saline. Thereafter, with the second and third gating devices 135, 132 closed while the fourth gating device 133 is opened to atmospheric pressure the liquid reservoir 110 is now filled with the saline at atmospheric pressure. In FIG. 5B the positive pressure pulse generator mechanism 180 (e.g., displaceable plunger) is arranged along the flexible vacuum pressure inlet tubing 120 proximally of the first and third gating devices 130, 132 as well as the liquid reservoir 110, whereas in FIG. 5C the positive pressure pulse generator mechanism 180 (e.g., displaceable plunger) is disposed with the first and third gating devices 130, 132 on the proximal side thereof while the liquid reservoir 110 is disposed on the distal side thereof.

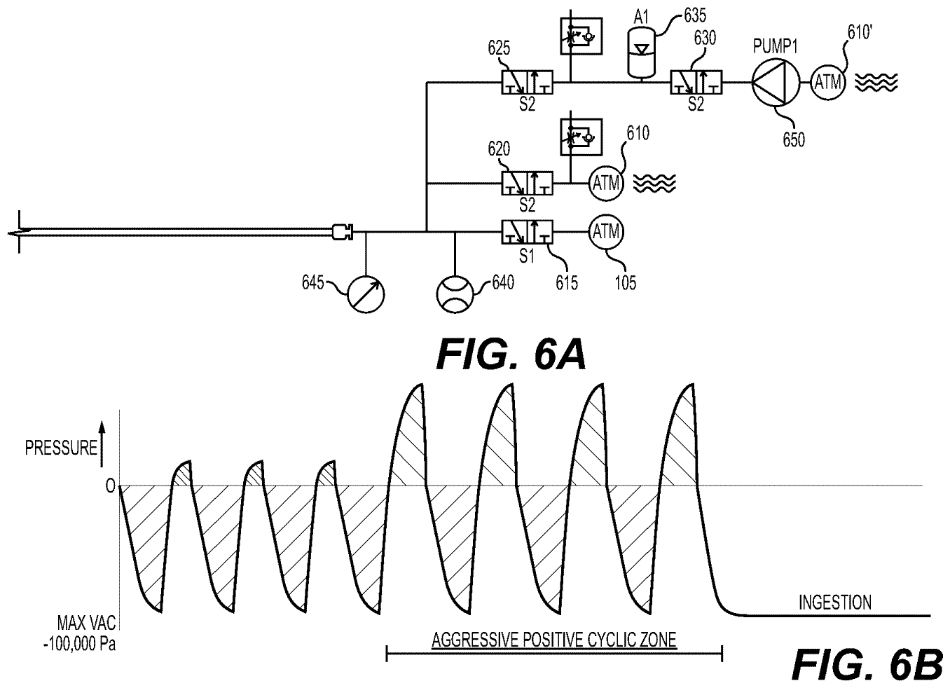

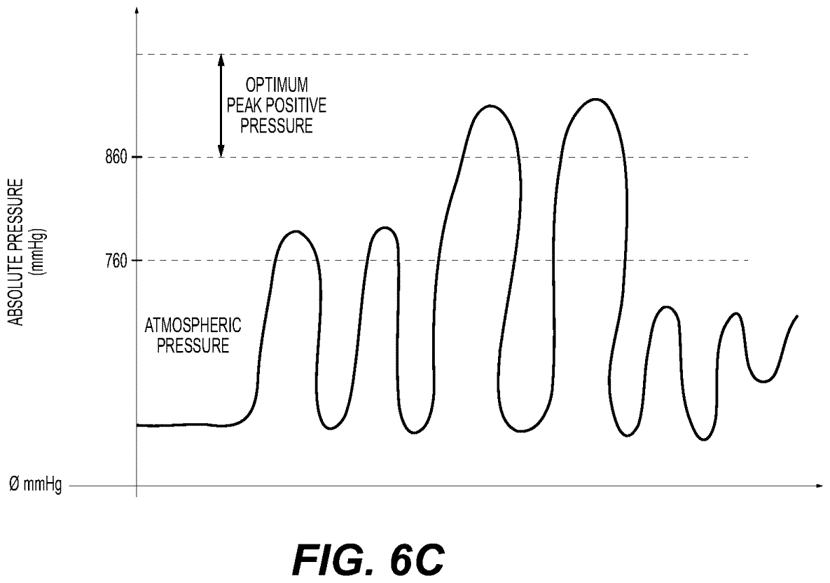

When producing a cyclic aspiration pressure waveform using a cyclic aspiration system, regardless of the type of system and thus manner in which the positive pressure pulse is generated, it may be advantageous after a predetermined number of cycles to increase in amplitude the generated positive pressure pulse (i.e., a period of heightened, enhanced, or more aggressive positive pressure) to aid with clot movement at the distal tip/end of the aspiration catheter. By heightening, enhancing, or increasing the amplitude of the positive pressure the extent to which the clot is aggressively expelled (i.e., punched) distally from the distal tip/end of the aspiration catheter allows for slight reorientation and/or change of shape (e.g., elongation) of the clot aiding in ingestion into the aspiration catheter during a subsequent cycle or pulse of vacuum pressure. FIG. 6A diagrammatically depicts an exemplary schematic diagram of a modified vented cyclic aspiration system vented to two liquid reservoirs open to atmospheric pressure for producing respectively a regular (non-heightened) positive pressure pulse as well as an increased “pumped-up”, heightened, or more aggressive positive pressure pulse. The vented cyclic aspiration system inlet tubing is connected in fluid communication between the vacuum pump 105 and the proximal hub 127 of the aspiration catheter 123. In FIG. 6A the inlet tubing has three separate inputs and one or more gating devices associated with each input. A first input of the inlet tubing has associated therewith a first gating device 615 controlling passage of the vacuum pressure generated by the vacuum pump 605. While a second input of the inlet tubing has associated therewith a second gating device 620 controlling therethrough a first (non-heightened) positive pressure generated by venting to a first liquid reservoir 610 open to atmospheric pressure. Lastly, a third input of the inlet tubing has associated therewith a third gating device 625, an accumulator 635, a fourth gating device 630, and a pump 650 connected to a second liquid reservoir 610′ open to atmospheric pressure, together producing the heightened (“aggressive”) positive pressure. The accumulator 635 may be a bladder/separator with pressurized air in the upper/top portion. Liquid (e.g., saline) pressurized by the pump 186 fills the lower/bottom portion compressing the bladder. The third and fourth gating device 625, 630 on respective sides of the accumulator 635 are then closed. When the third gating device 625 is opened, a rush of pressurized saline enters the system causing the pressure to surge creating the heightened positive pressure. The fourth gating device 630 acts as a flow control valve to adjust or control the extent or level of the surge. Alternatively, a pressurized syringe may replace the bladder as the accumulator. In operation, at any given time, only one of the gating devices 615, 620, 625 is open to allow flow therethrough and into the aspiration catheter of the vacuum pressure, regular (i.e., non-heightened) positive pressure, or heightened positive pressure. Accordingly, the accumulator 635 in the cyclic aspiration system in FIG. 6A may be used to control (e.g., boost, heighten, or increase) the amplitude of the positive pressure internal of the cyclic aspiration pressure wave on demand. The height or amplitude of the wave may be varied or controlled based on the length of time the gating device 625 is opened to release the energy stored in the accumulator 635.

FIG. 6B is an exemplary representative pressure waveform over time depicting seven cycles with each cycle undergoing an interval of vacuum pressure followed by a positive pressure interval. In the exemplary representative pressure waveform, the downward spikes represent intervals of vacuum pressure while the upward spikes denote the positive pressure intervals of the cyclic aspiration pressure waveform. The first three cycles depict a constant amplitude of vacuum pressure followed by a constant first (i.e., regular or non-heightened) amplitude of the positive pressure. It is during these initial three cycles that the clot is ingested proximally into the aspiration catheter when subject to vacuum pressure and displaced in a distal direction while remaining within the distal section of the aspiration catheter (i.e., without exiting or being expelled from the distal tip/end) when subject to the regular (i.e., non-heightened) positive pressure. Referring once again to the cyclic aspiration pressure waveform in FIG. 6B, the last three cycles maintain the same constant amplitude of vacuum pressure followed by a constant second amplitude of heightened positive pressure greater than the constant first (i.e., regular, or non-heightened) amplitude positive pressure. The second constant amplitude of the heightened positive pressure is sufficient to slightly expel or eject the clot from the distal tip/end of the aspiration catheter, before being drawn back into the distal end/tip of the aspiration catheter during the next vacuum pressure interval. With each cycle of alternating vacuum pressure and positive pressure pulse at the second constant amplitude of heightened positive pressure the clot can slightly reorient and/or change shape (e.g., elongate) aiding or assisting in ingestion of the clot. Referring to FIG. 6B, the slope of the pressure waveform is more vertical when cycling from vacuum pressure to positive pressure when the enhanced, aggressive, heightened, or increased positive pressure is injected in comparison to the slope during the regular or non-heightened positive pressure (i.e., without the increased positive pressure).

It is recognized that there may be an optimum positive pressure (i.e., optimum high pressure or peak pressure) for the cyclic aspiration pressure waveform. Such optimum positive pressure is preferably within the range denoted between the bidirectional arrows in the exemplary graphical representation in FIG. 6C. Preferably the optimum positive pressure range is between approximately 5 kPa and approximately 200 kPa above atmospheric pressure (760 mmHg). More preferably the optimum pressure range is between approximately 5 kPa and approximately 100 kPa above atmospheric pressure (760 mmHg). While most preferably, the optimum positive pressure range is between approximately 20 kPa and approximately 80 kPa above atmospheric pressure (760 mmHg). Advantageously, this optimum positive pressure (i.e., peak pressure) is slightly above the patient's blood pressure so that the clot is ever so slightly released from the distal tip/end of the aspiration catheter before being aspirated (i.e., sucked) rapidly back into the aspiration catheter in each cycle. As described above, the clot may slightly reorient and change its shape (e.g., elongate) with each pulse or cycle. Selecting too high a maximum positive pressure risks losing the clot altogether or injecting it more distally into the vessel. Whereas if the maximum positive pressure selected is too low the clot is not able to reorient between pulses and the same portion of the clot face is continuously hammered by engaging with the distal tip/end of the aspiration catheter.

Those exemplary cyclic aspiration systems described above that generate the positive pressure pulse within inlet tubing (e.g., vacuum inlet tubing and/or positive pressure inlet tubing) disposed proximally of the proximal hub attached to the aspiration catheter employ one or more gating devices that become contaminated by blood during use. Once exposed and contaminated with blood, these gating devices are prone to clogging. To minimize the potential for clogging, it is desirable to dispose or discard the gating devices after a single use. However, electrical components associated with actuating conventional gating devices (e.g., valves or solenoids) are expensive and thus not to be discarded after a single use. It is therefore desirable to develop an improved gating device in which expensive components are not contaminated by blood, reusable and separate from those components contaminated by blood, less costly, and discardable after a single use. Specifically, the two types of components representing the gating device in accordance with the present disclosure include actuator components (not contaminated by blood during use) operating non-actuator components (contaminated by blood during use). Actuator components are components requiring power or energy to operate; whereas non-actuator components are mechanical components rather than requiring power or energy on their own instead are operated (i.e., displaced) via the actuator components. Since the non-actuator components are contaminated by blood during use only inexpensive components are utilized and discarded after a single use, whereas those actuator components more costly to manufacture are reusable because they are specifically arranged in the system to avoid contamination by blood. Countless examples of gating devices are possible to achieve this desired goal of which several non-limiting examples are shown and described in detail below.

Referring to FIGS. 10A-10D is an exemplary gating device in accordance with the present disclosure comprising both non-actuator components contaminated by blood and actuator components not contaminated by blood, wherein the non-actuator components and actuator components are separable from one another. After a single use, the non-actuator components that are contaminated by blood are discarded minimizing the risk of potential clogging, while the more expensive actuator components are not contaminated by blood and thus reusable. In the example in FIGS. 10A-10D the non-actuator components contaminated by blood include the inlet tubing 120, housing 1015, and an internal displaceable member 1020, while the actuator components that are not contaminated by blood include a pair of electromagnets 1005, 1010. Housing 1015 is preferably made of a molded polymer as either a single unitary integral component or multiple sections securable together to form a single component. Along a portion of the inlet tubing 120 is a recess matching at least in part a shape of the internal displaceable member 1020. In the example shown in FIGS. 10A-10D, the internal displaceable member 1020 is a ball having an associated conductive element (e.g., a conductive metal strip) while the corresponding recess is substantially hemispherical matching in size and shape with that of the ball (as shown in FIG. 10A without the ball). The size and shape of the recess and corresponding internal displaceable member being selected so that when the internal displaceable member is seated in the recess unblocked or unobstructed passage therethrough, whereas passage therethrough is blocked or occluded when the internal displaceable member is unseated from the recess. The actuator components, e.g., a pair of electromagnets 1005, 1010, are arranged externally of the inlet tubing 120 thereby never being exposed to or contaminated by the blood during use. FIGS. 10B and 10C illustrate the gating device in the open state permitting unblocked maximum passage therethrough and the blocked or occluded state prohibiting passage therethrough, respectively, in response to energizing only one of the pair of electromagnets 1005, 1010 at any given time. Specifically, in response to energizing the first electromagnet 1005 (while the second electromagnet 1010 remains deenergized), the ball 1020 is drawn thereto (i.e., raised) and seated in the corresponding recess defined in the inlet tubing 120 allowing unblocked maximum passage therethrough (FIG. 10B). Whereas, when the second electromagnet 1010 is energized (while the first electromagnet 1005 is deenergized) the ball 1020 is drawn thereto and unseated from the corresponding recess defined in the inlet tubing 120 obstructing or blocking passage therethrough (FIG. 10C). After a single use, the non-actuator components (e.g., the inlet tubing 120, the housing 1015 and the internal displaceable member 1020) contaminated by blood are discarded, preferably as an assembled unit or module (as denoted by an arrow pointing to a trashcan), while the actuator components (e.g., the pair of electromagnets 1005, 1010) not exposed to or contaminated by the blood are reusable (as denoted by the electromagnets 1005, 1010 remaining stationary in position), as depicted in FIG. 10D. Ball 1020 in FIGS. 10A-10D functions as a valve, however, it is noted that the ball 1020 if made into a ball and plunger shape could be used as a positive pressure pulse generator mechanism to create the positive pressure pulse in a vented cyclic aspiration system.

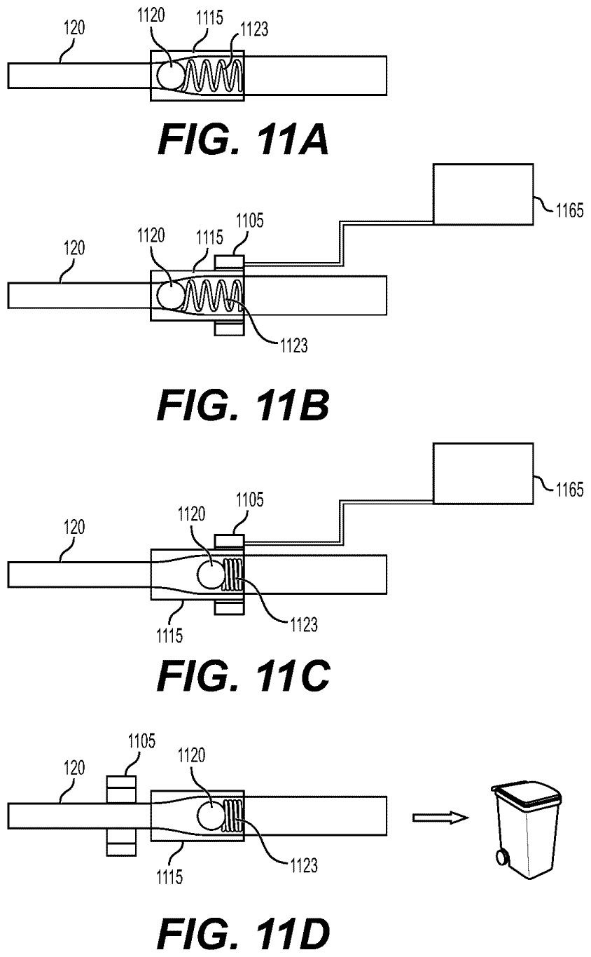

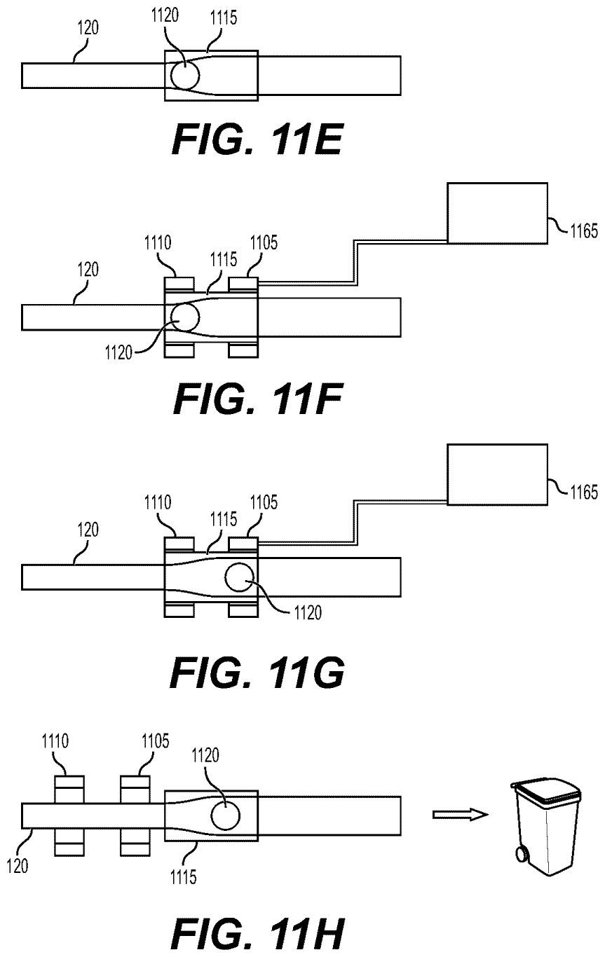

Another exemplary gating device in accordance with the present disclosure is found in FIGS. 11A-11D to include both non-actuator components contaminable by blood and actuator components not contaminated by blood, wherein the non-actuator and the actuator components are separable from one another. The non-actuator components (e.g., inlet tubing 120, a housing 1115, an internal displaceable member 1120, and an internal compressible spring 1123) are contaminated by blood; while the actuator component (e.g., circular electromagnet 1105) is arranged externally of the inlet tubing 120 so as not to be contaminated by blood and hence reusable. A portion of the lumen of inlet tubing 120 aligned with that of the housing 1115 has an inner diameter tapered from a wide end to an opposite narrow end. Disposed within the tapered inner diameter of the inlet tubing 120 is the internal displaceable member 1120 (e.g., a ball with an associated conductive element such as conductive metal strip). While the electromagnet 1105 is deenergized, the internal compressible spring 1123 is in a default axially non-compressed state fully extended in an axial direction maintaining the ball 1120 seated in the narrow end of the tapered inner diameter of the vacuum inlet tubing 120 blocking and occluding passage therethrough (FIG. 11B). In response to a controller 1165 (e.g., processor) energizing the electromagnet 1105, the ball 1120 is drawn thereto (i.e., towards the wider end of the tapered inner diameter of the inlet tubing 120) axially compressing the spring 1123 and allowing unblocked maximum passage therethrough (FIG. 11C). After a single use, the non-actuator components (e.g., the inlet tubing 120, the internal displaceable member 1120, the housing 1115, and the internal compressible spring 1123) contaminated by blood are discarded (as denoted by an arrow pointing to a trashcan), preferably as an assembled unit or module. Whereas, the actuator component (e.g., the electromagnet 1105) arranged externally of the inlet tubing 120 and thus not contaminated by blood is reusable (as denoted by the electromagnet 1105 remaining stationary in position while the inlet tubing 120, the housing 1115, and internal displaceable member 1120 as an assembled unit or module are moved towards the trashcan), as depicted in FIG. 11D.

FIGS. 11E-11H is yet another example of the gating device in accordance with the present disclosure. This example in FIGS. 11E-11H represents a modification of the previous example in FIGS. 11A-11D. In comparing these examples, the differences therebetween being that the internal compressible spring 1123 is eliminated and a second electromagnet 1110 is added in the example of FIGS. 11E-11H to axially displace the internal displacement member within the tapered inner diameter of the lumen of the inlet tubing. As with the previously described examples, the example gating device in FIGS. 11E-11F includes both non-actuator components contaminated by blood and actuator components not contaminated by blood, wherein the actuator and non-actuator components are separable from one another. In FIGS. 11E-11H the non-actuator components contaminated by blood include inlet tubing 120, a housing 1115, and an internal displaceable member 1120; while the actuator components not contaminated by blood include two circular electromagnets (a first electromagnet 1110 and a second electromagnet 1105) with only one electromagnet being energized at any given time. A portion of the lumen of the inlet tubing 120 aligned with that of the housing 1115 has an inner diameter tapered from a wide end to an opposite narrow end (as shown in FIG. 11E depicting the gating device without the two electromagnets). Disposed within the tapered inner diameter section of the lumen of the inlet tubing 120 is the internal displaceable member 1120 (e.g., a ball with an associated conductive element such as conductive metal strip). In response to a controller 1165 (e.g., processor) energizing the second electromagnets 1110 closest to the narrow end of the tapered inner diameter of the vacuum inlet tubing 120 (while the first electromagnet 1105 closest to the widest end of the tapered inner diameter of the vacuum inlet tubing 120 remains deenergized) drawing the ball 1120 to the narrow end of the tapered inner diameter of the vacuum inlet tubing 120 obstructing and blocking passage therethrough (FIG. 11F). In response to the controller 1165 (e.g., processor) energizing the second electromagnet 1105, the ball 220 due to the conductive strip associated therewith is drawn thereto towards the wider end of the tapered inner diameter of the inlet tubing 120 allowing unblocked maximum passage therethrough (FIG. 11G). After a single use, the non-actuator components contaminated by blood (e.g., the inlet tubing 120, the housing 1115, and the internal displaceable member 1120) as an assembled unit or module are discarded (as denoted by an arrow pointing to a trashcan) while actuator components (e.g., the two electromagnets 1105, 1110) not contaminated by the blood are reusable (as denoted by the two electromagnets remaining stationary in position while the inlet tubing 120, housing 1115 and displaceable member 1120 are moved towards the trashcan), as depicted in FIG. 11H.

The hybrid cyclic aspiration system in FIG. 3A employs as the inlet tubing a single inlet tube having a single lumen defined therein in a longitudinal/axial direction from the proximal end connected to the vacuum pump to the opposite distal end connected to the aspiration catheter via the proximal hub. Subject to the generated cyclic aspiration pressure waveform, collected fluid (e.g., saline and/or blood together with formed bubbles and captured clots present therein) in the cyclic aspiration system travels bi-directionally through the single lumen of the single inlet tube. Specifically, the collected fluid (i.e., collected liquid (e.g., saline and/or blood) together with formed bubbles and captured clots present therein) in the single lumen of the single inlet tube travels in the proximal direction in response to the vacuum pressure produced by the vacuum pump and in the distal direction in response to the positive pressure pulse produced by the positive pressure pulse generator mechanism (while passage of the vacuum pressure generated by the vacuum pump is blocked via the vacuum pressure gating device). As a result, when subject to the cyclic aspiration pressure waveform, the collected fluid (i.e., collected liquid (e.g., saline and/or blood) together with formed bubbles and captured clots present therein) in the bi-directional hybrid cyclic aspiration system of FIG. 3A remains trapped in (i.e., never exiting from) the system. To optimize efficiency of capture of the clot the cyclic aspiration pressure waveform cycles between intervals of positive pressure and vacuum pressure at a relatively high cyclic rate (preferably in a range of approximately 1 Hz to approximately 20 Hz). Cycling between vacuum pressure and positive pressure changes the pressure of the collected fluid in the system from vacuum pressure to a positive pressure with each cycle resulting in bi-directional (i.e., back-and-forth movement) travel of the collected fluid in the single lumen of the single inlet tube. As described in detail above, during cycling between vacuum pressure and positive pressure formed bubbles in the collected fluid become trapped in (i.e., never exiting from) the single lumen of the single inlet tube. These trapped bubbles undesirably dampen or decay the produced cyclic aspiration pressure wave applied at the distal tip of the aspiration catheter resulting in discontinuity and inconsistency in cyclic movement of the to be captured clot subject thereto thereby hindering capture. Moreover, because the captured clots are trapped in (i.e., never exit or ejected from) the single lumen of the single inlet tubing in the bi-directional hybrid cyclic aspiration system of FIG. 3A is at heightened risk of clogging.

To optimize continuity and consistency of the cyclic aspiration pressure waveform applied to the distal end of the aspiration catheter thereby fostering clot capture, it is therefore desirable to develop an improved hybrid cyclic aspiration system that allows for the escape or exit of the formed bubbles in the system. In connection therewith, it is further desirable to restore liquid to the system in response to that depleted during aspiration of the collected fluid (i.e., collected liquid (e.g., saline and/or blood) together with the formed bubbles and captured clots present therein). These goals are realized by configuring the hybrid cyclic aspiration system as part of a continuous loop or pathway whereby the collected fluid (i.e., collected liquid (e.g., saline and/or blood) together with the formed bubbles and captured clots present therein) when subject to the cyclic aspiration pressure waveform travels unidirectionally via separate lumen, rather than bi-directionally back-and-forth via a single lumen. During the vacuum pressure interval, the collected fluid (i.e., collected liquid (e.g., saline and/or blood) together with the formed bubbles and captured clots present therein) are ejected (i.e., exit or purged) from the system when aspirated (i.e., sucked) into the vacuum collection vessel 1305a associated with the vacuum pump 1305. As part of the continuous loop, the liquid in the system that is depleted or lost when the collected fluid is aspirated into the vacuum collection vessel 1305a is replenishable (i.e., restored) with the liquid in the liquid reservoir open to the atmosphere 1310. A stage or interval of replenishment of the liquid in the system may occur on demand or once every N cycles, wherein each cycle includes a positive pressure interval and a vacuum pressure interval, with N being a positive integer greater than 0.

Unless refilled, over time replenishing the aspirated collected liquid in the unidirectional continuous loop hybrid cyclic aspiration system exhausts the pre-filled (i.e., prior to producing the cyclic aspiration pressure waveform) volume of liquid in the liquid reservoir open to the atmosphere. In accordance with the present disclosure, refilling (e.g., restoring to the original pre-filled volume) of the liquid in the liquid reservoir subject to the atmosphere is also preferably included as part of the continuous loop. That is, the liquid reservoir subject to the atmosphere 1310 is preferably refillable by recirculating the aspirated (i.e., sucked) collected fluid (i.e., collected liquid (e.g., saline and/or blood) together with the formed bubbles) stored in the vacuum collection vessel 1305a associated with the vacuum pump 1305. The captured clots present in the vacuum collection vessel 1305a are periodically removed and thus not recirculated back into the system. Escape from the vacuum collection vessel 1305a of any captured clots yet to be removed is prohibited via a filter associated therewith. When the stored collected fluid (i.e., collected liquid (e.g., saline and/or blood) together with the formed bubbles present therein, but excluding the captured clots) from the vacuum collection vessel 1305a is received in the liquid reservoir open to the atmosphere 1310 the formed bubbles therein automatically escape into the atmosphere so that what remains in the liquid reservoir 1310 is only the degassed liquid (i.e., absent or free of the formed bubbles) to be cycled back into the unidirectional continuous loop hybrid cyclic aspiration system during the next replenishment interval.

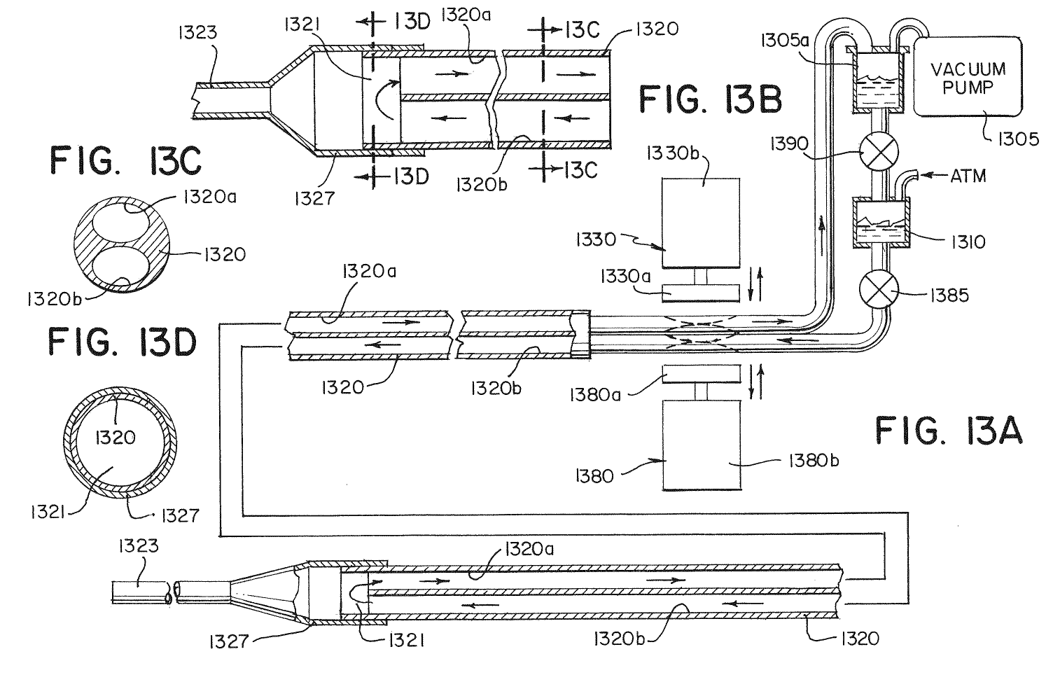

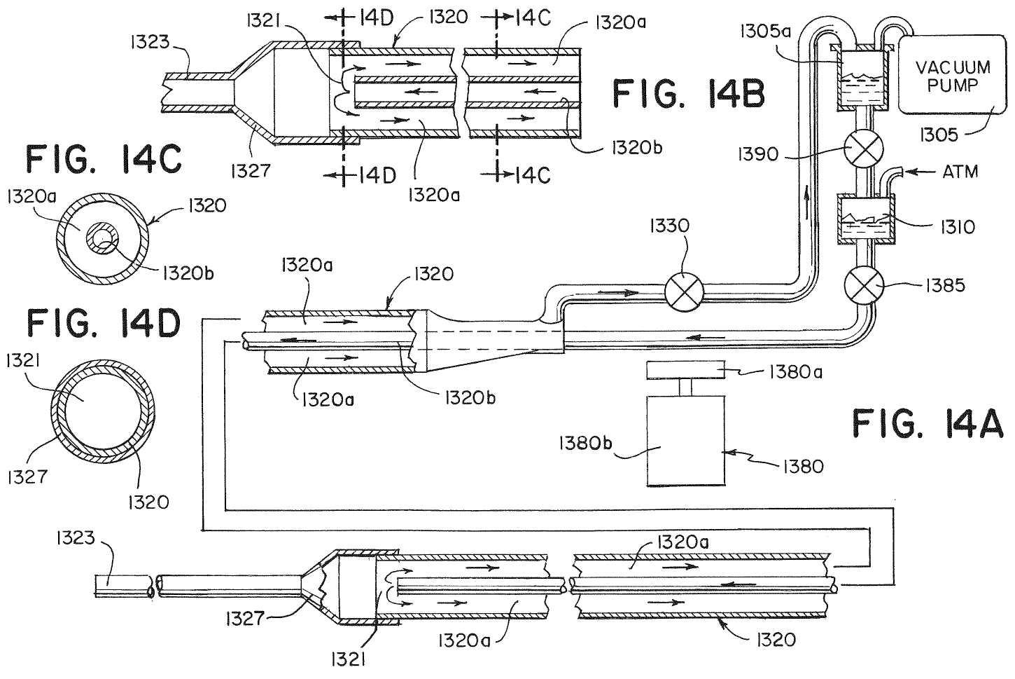

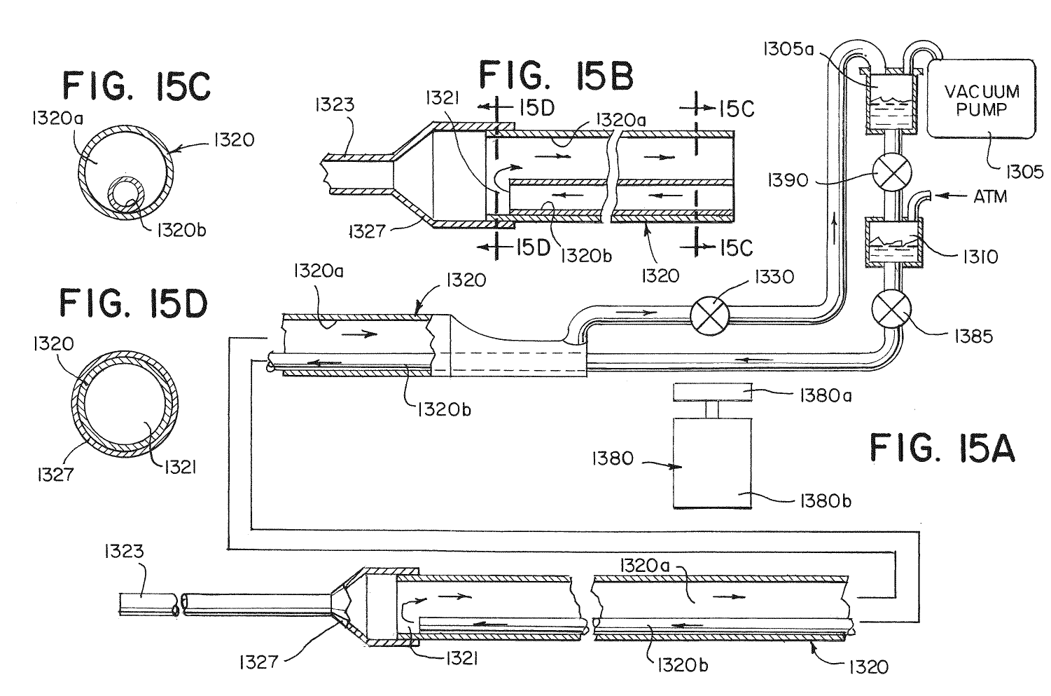

FIG. 13A is a schematic diagram of an example unidirectional continuous loop hybrid cyclic aspiration system in accordance with the present disclosure. Starting at the distal end, the system includes an aspiration catheter 1323 with an associated catheter hub 1327 disposed on the proximal end thereof. Proximally of the catheter hub 1327 is a single inlet tube 1320 having defined therein a vacuum pressure lumen 1320a and a separate positive pressure lumen 1320b with each extending in a longitudinal/axial direction substantially parallel to one another. Different arrangements of the respective vacuum pressure and positive pressure lumen 1320a, 1320b, respectively, of the single inlet tube 1320 are contemplated and within the intended scope of the present disclosure. In the example of FIG. 13A, the vacuum pressure and positive pressure lumen 1320a, 1320b, respectively, are arranged side-by-side extending parallel to one another in a longitudinal/axial direction in the single inlet tube 1320, as shown in the radial cross-sectional view in FIG. 13C. Alternatively, the vacuum pressure and positive pressure lumen 1320a, 1320b, respectively, of the single inlet tube 1320 may be arranged concentrically (FIGS. 14A-14D), or eccentrically (FIGS. 15A-15D). Other arrangements of the vacuum pressure and positive pressure lumen 1320a, 1320b, respectively, defined within the single inlet tube 1320 are within the scope of the present disclosure.

Collected fluid (i.e., collected liquid (e.g., saline and/or blood) together with the formed bubbles and captured clots present therein) in the inlet tubing 1320 is subject to travel along a continuous pathway in response to the cyclic aspiration pressure waveform produced by the interactive operation of the vacuum pressure pump 1305, the vacuum pressure gating device 1330, and the positive pressure pulse generator mechanism 1380. Operation of these devices to produce the respective positive pressure pulse interval and vacuum pressure interval of the cyclic aspiration pressure waveform is described in further detail. The positive pressure interval of the cyclic aspiration pressure waveform represents a two-stage process. In a first stage, the vacuum pressure gating device 1330 (e.g., pinch valve, pin, plunger, etc. and associated actuator, solenoid, or any of the gating devices described herein) is transitioned to the closed state preventing, blocking, or occluding passage therethrough of the vacuum pressure generated by the vacuum pump 1305 into the vacuum pressure lumen 1320a. Blocking or occluding the vacuum pressure in the vacuum pressure lumen 1320a allows build-up of positive pressure in the positive pressure lumen 1320b during a second or subsequent stage in which the positive pressure pulse is created. By way of illustrative example in FIG. 13A, the vacuum pressure gating device 1330 is an externally arranged plunger 1330a displaceable (i.e., advanced or retracted) via an associated actuator 1330b. In an advanced state, the plunger 1330a externally compresses a flexible or compressible proximal section (hereinafter referred to as a “flexi section”) (denoted in FIG. 13A by the dashed semicircular curved lines while in a radially compressed/collapsed state) of the inlet tubing 1320 blocking passage through the vacuum pressure lumen 1320a. Some gating devices described herein operate by internally occluding, rather externally radially compressing, the inlet tubing thereby eliminating the need for the flexi section of the inlet tubing associated with the vacuum pressure lumen 1320a. For instance, a ball internally disposed within the lumen of the inlet tubing is displaceable between an open position allowing passage therethrough and a closed position occluding passage therethrough via externally arranged electromagnets. Creation of the positive pressure pulse in the second stage is realized using any of the positive pressure pulse generator mechanisms described herein (e.g., external plunger 1380a displaceable (i.e., advanced or retracted) via an associated actuator 1380b). In an advanced state, the plunger 1380a externally compresses a flexible or compressible proximal section (hereinafter referred to as a “flexi section”) (denoted in FIG. 13A by the dashed semicircular curved lines while in a radially compressed/collapsed state) of the inlet tubing blocking passage through the positive pressure lumen 1320b reducing the internal volume displacing the collectable fluid therein thereby creating the positive pressure pulse. During the vacuum pressure interval the vacuum pressure gating device 1330 (e.g., plunger 1330a) is in an open state allowing unrestricted passage therethrough of the vacuum pressure generated by the vacuum pump 1305 aspirating the collected fluid (i.e., collected liquid (e.g., saline and/or blood) together with the formed bubbles and captured clots present therein) unidirectionally in the proximal direction through the vacuum pressure lumen 1320a into the vacuum collection vessel 1305a.

While each of the plungers 1380a, 1330a are in a retracted state, the flexi section of the positive pressure lumen 1320b and vacuum pressure lumen 1320a, respectively, automatically reverts to its original, natural, default, radially non-compressed state (as represented by the solid straight line). Preferably, only a proximal section (e.g., flexi section) of the inlet tubing 1320 associated with the positive pressure lumen 1320b (and optionally the vacuum pressure lumen 1320a when the vacuum gating device 1330 employed (e.g., pinch valve or plunger) operates by externally radially compressing the vacuum pressure lumen 1320a) is made of a flexible or compressible material. That remaining portion (i.e., non-flexi section) of the inlet tubing 1320 disposed distally of the flexi section is preferably less flexible (i.e., more rigid) relative thereto. Otherwise, manufacture of the entire length (i.e., from the proximal end to the opposite distal end) of the inlet tubing using a flexible/compressible material that expands/contracts in response to the cyclic aspiration pressure wave would undesirably absorb and diminish the cyclic aspiration pressure waveform applied at the distal end of the aspiration catheter reducing efficiency of capture of the clot.

To form the continuous loop or pathway, sufficient clearance space is provided to allow travel of the collected fluid (i.e., collected liquid (e.g., saline and/or blood) together with the formed bubbles and captured clots present therein) from the distal end of the positive pressure lumen 1320b into the distal end of the vacuum pressure lumen 1320a. Clearance space 1321 may be provided in a distal section of the single inlet tube itself 1320 and/or in the catheter hub 1327 connected to the distal end thereof. FIG. 13B is an enlarged view of the connection between the proximal end of the catheter hub 1327 and the distal end of the single inlet tube 1320 of the example unidirectional continuous loop hybrid cyclic aspiration system of FIG. 13A. The clearance space 1321 in FIG. 13B is provided within the single inlet tube 1320 itself, proximally of its distal tip. While undergoing repeated cycling from the positive pressure interval to the vacuum pressure interval, the clearance space 1321 provides a pathway for continuous travel (i.e., turn around) of the collected fluid (i.e., collected liquid (e.g., saline and/or blood) together with the formed bubbles and captured clots present therein) exiting from the distal end of the positive pressure lumen 1320b to enter the distal end of the vacuum pressure lumen 1320a. Similar clearance space 1321 in a distal section of the single inlet tube 1320 is represented for the dual lumen arranged concentrically (FIG. 14B) and eccentrically (FIG. 15B).

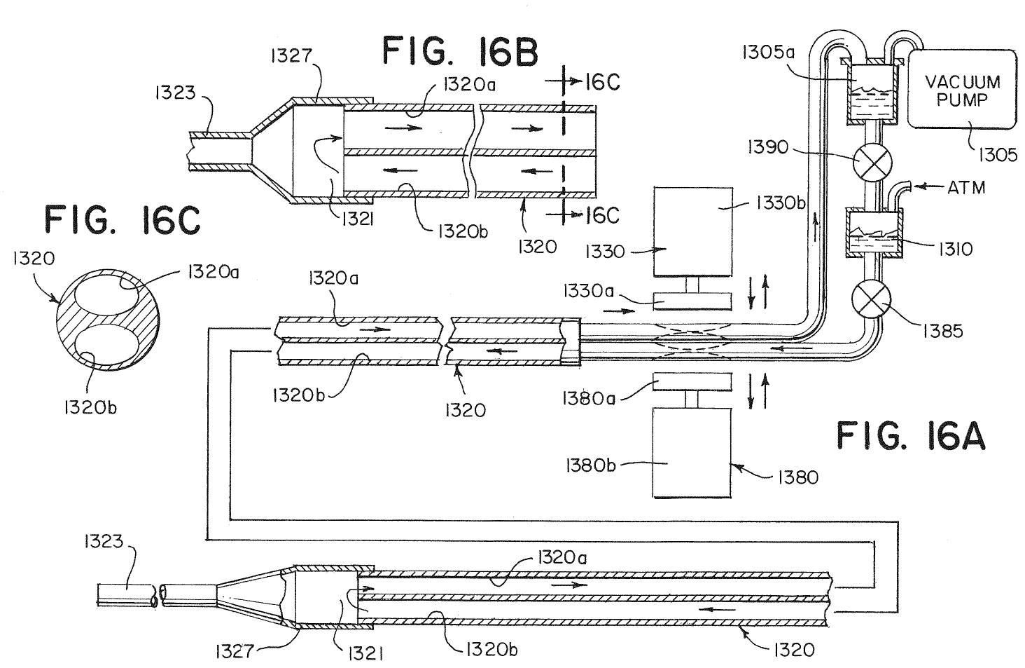

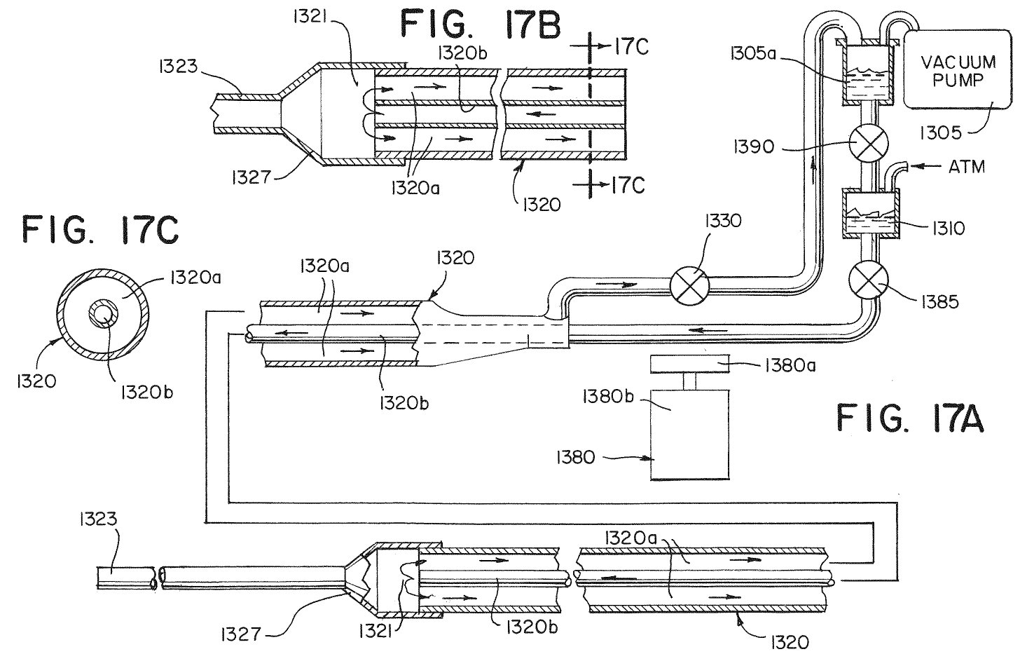

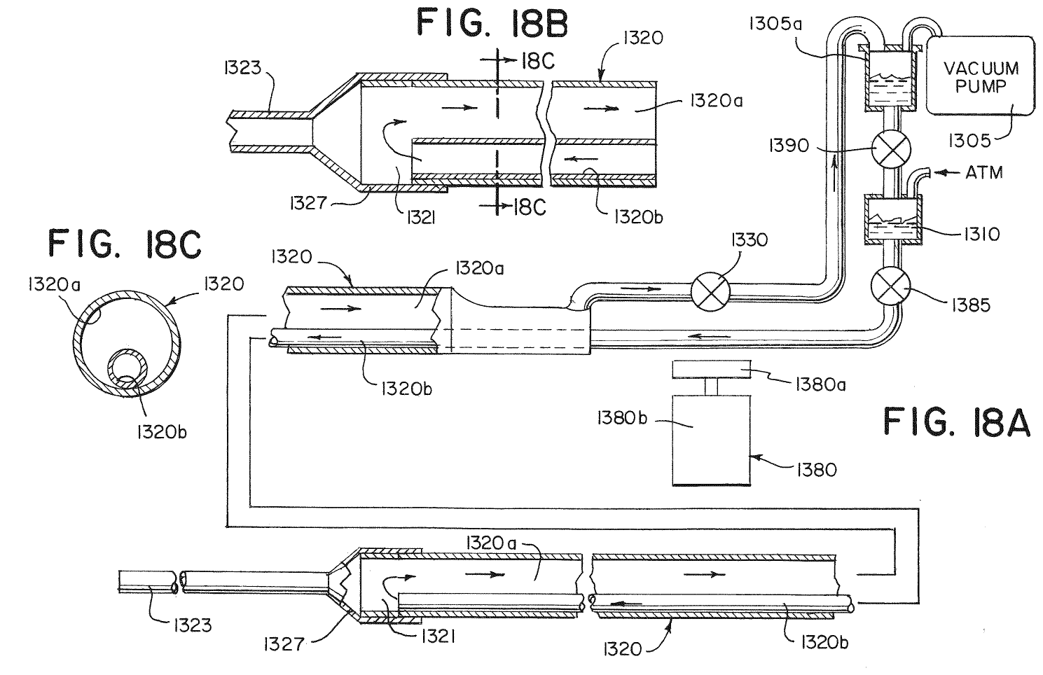

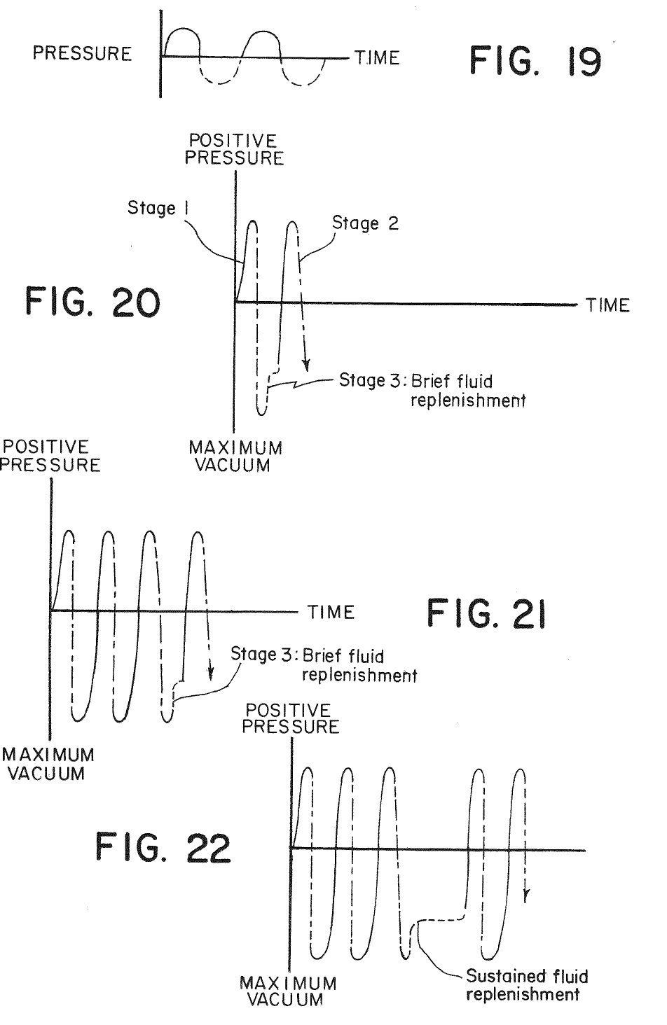

To simplify manufacture, during extrusion of the single inlet tube 1320 the respective vacuum pressure lumen 1320a and positive pressure lumen 1320b may extend from the proximal end to the distal end/tip. With this arrangement, the clearance space 1321 allowing continuous travel (i.e., turnaround) of the collected fluid in the system after exiting the distal end of the positive pressure lumen 1320b to enter the distal end of the vacuum pressure lumen 1320a is provided exclusively in the catheter hub 1327 (FIG. 16B—example of dual lumen arranged side-by-side). Similar travel (i.e., turnaround) clearance areas 1321 disposed in the catheter hub 1327 are illustrated for the exemplary concentrically arranged dual lumen (FIG. 17B) and eccentrically arranged dual lumen (FIG. 18B). Irrespective of whether arranged in the inlet tubing 1320 and/or in the catheter hub 1327, the clearance space 1321 represents part of the continuous pathway of unidirectional travel of the collected fluid after exiting from a distal end of the positive pressure lumen 1320b prior to entering the distal end of the vacuum pressure lumen 1320a.

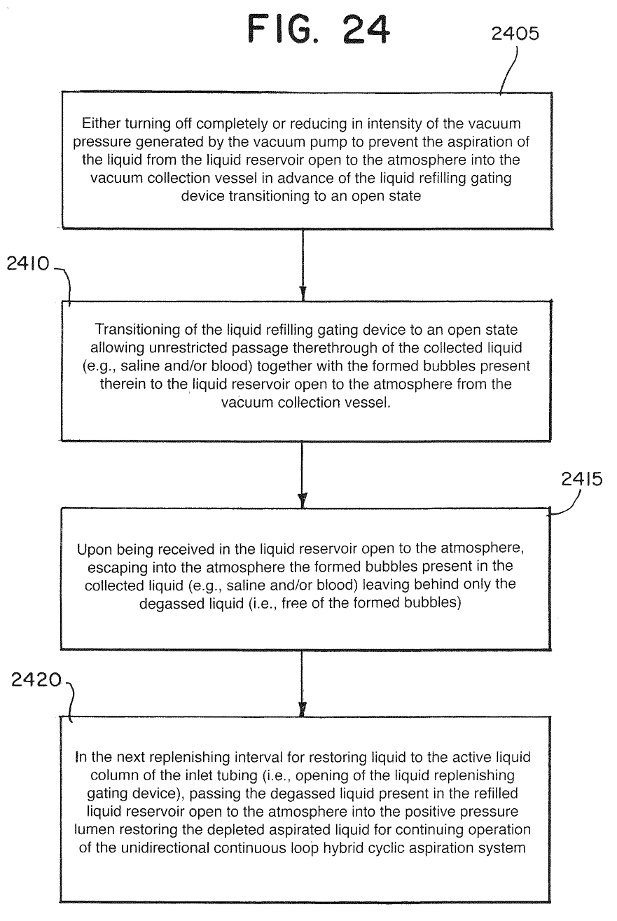

In lieu of a single inlet tube having two separate lumen (i.e., a vacuum pressure lumen 1320a and a positive pressure lumen 1320b) defined therein as shown in FIG. 16A, the active liquid channel may comprise two inlet tubes separate from one another with their respective distal ends connected to the catheter hub 1327, as shown in FIGS. 24A-24C. Each inlet tube has defined therein a single lumen. That is to say, the vacuum pressure inlet tube has defined therein a single lumen (i.e., vacuum pressure lumen) and the separate positive pressure inlet tube has defined therein a single lumen (i.e., positive pressure lumen).

Regardless of the number of inlet tubes (e.g., a single inlet tube or two separate inlet tubes, as described further below) and arrangement (e.g., side-by-side, concentric, or eccentric) of the vacuum pressure lumen and positive pressure lumen, when subject to the cyclic aspiration pressure waveform the collected fluid (i.e., collected liquid (e.g., saline and/or blood) together with the formed bubbles and captured clots present therein) travels in only one direction unidirectionally (rather bi-directionally back-and-forth) through each lumen. Moreover, unidirectional travel exclusively in the proximal direction through the vacuum pressure lumen 1320a is opposite to the unidirectional travel exclusively in the distal direction through the positive pressure lumen 1320b forming a continuous loop or pathway. Furthermore, the vacuum pressure lumen (i.e., vacuum pressure line) and the positive pressure lumen (i.e., positive pressure line) represent separate lines or pathways with passage through each being independently controllable (i.e., blockable) of one another.

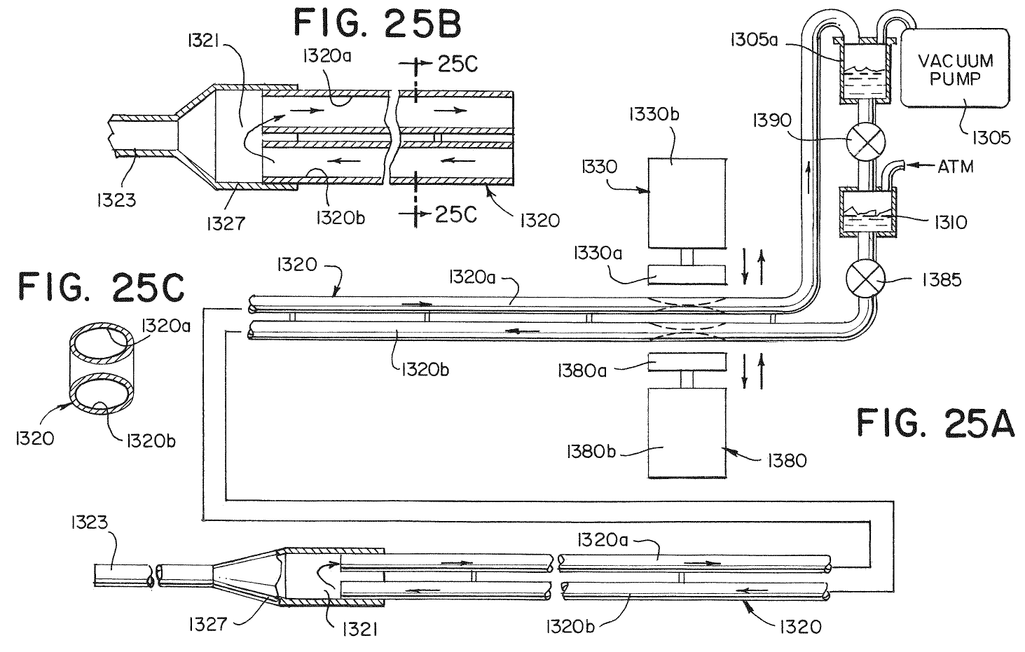

The respective vacuum pressure lumen 1320a and positive pressure lumen 1320b may, but need not necessarily be, equal in inner diameter to one another. If not equal in inner diameter, either the vacuum pressure lumen or the positive pressure lumen may be larger relative to that of the other. In the exemplary side-by-side arrangement in FIG. 13C the respective vacuum pressure lumen 1320a and positive pressure lumen 1320b are substantially equal in inner diameter. However, in the example concentric arrangement in FIGS. 14C & 17C the positive pressure lumen 1320b has a smaller inner diameter relative to that of the inner diameter of the vacuum pressure lumen 1320a. Similarly, in the exemplary eccentric arrangement in FIGS. 15C & 18C, the inner diameter of the vacuum pressure lumen 1320a is greater than the inner diameter of the positive pressure lumen 1320b. In the exemplary two separate inlet tubes configuration in FIG. 25A the inner diameter of the vacuum pressure lumen 1320a and the positive pressure lumen 1320b are substantially equal.

Repeating cycles of intermittent intervals of vacuum pressure and positive pressure produce the cyclic aspiration pressure waveform by which the captured clots traverse unidirectionally in a continuous loop or pathway through the active liquid channel from the positive pressure lumen 1320b into the vacuum pressure lumen 1320a. That is, collected fluid (i.e., the collected liquid (e.g., saline and/or blood) together with formed bubbles and captured clots present therein) during the positive pressure interval (i.e., creation of the positive pressure pulse via the positive pressure pulse generator mechanism 1330 while the vacuum pressure gating device 1330 is closed) travels unidirectionally in the distal direction through the positive pressure lumen 1320b. In response to the opening of the vacuum pressure gating device 1330 allowing unrestricted passage of the vacuum pressure therethrough, the aspirated collected fluid (i.e., the collected liquid (e.g., saline and/or blood) together with the formed bubbles and captured clots present therein) exits the distal end of the positive pressure lumen 1320b and via the clearance space 1321 enters the distal end of the vacuum pressure lumen 1320a traveling in a continuous loop or pathway. Therefore, the unidirectional travel through each of the lumen (1320a, 1320b) in opposite directions of one another together with the clearance space 1321 (either at the distal end of the inlet tube 1320 and/or proximal end of the catheter hub 1327) creates the continuous loop or pathway. It is during the vacuum pressure interval that aspirated collected fluid (i.e., the collected liquid (e.g., saline and/or blood) together with the formed bubbles and capture clots) is collected in the vacuum collection vessel 1305a. Counterproductively, the aspirated collected liquid received in the vacuum collection vessel 1305a depleted from the system undesirably fosters or promotes formation of new bubbles therein. Replenishing or restoring liquid (e.g., saline) back into the system to maintain a predetermined volume of liquid therein is therefore desirable to minimize the formation of new bubbles. The source for replenishing or restoring the volume of liquid in the system is the liquid in the liquid reservoir open to the atmosphere 1310 connected in fluid communication with the positive pressure lumen 1320b via a liquid replenishing gating device 1385 controlling blocking passage therethrough. In the schematic diagrams, the liquid replenishing gating device 1385 is depicted as an independently controllable gating device separate from the positive pressure pulse generator mechanism 1380 (e.g., plunger 1380a). Alternatively, the replenishing gating device 1385 may be integrated or combined with that of the positive pressure pulse generator mechanism 1380 (e.g., plunger 1380a) into a single unitary device in which the two devices are displaceable simultaneously at the same time in different stages of actuation depending on the extent (e.g., partial vs. full) of displacement (e.g., advancement or retraction). For example, the liquid replenishing gating device may be a lead in (e.g., pin) disposed on the proximal side and projecting upward from the contacting surface of the plunger 1380a as a single unitary device simultaneously displaceable together. During the positive pressure interval, the single unitary device is partially (initially or first stage) advanced such that the lead in (e.g., pin) radially compresses the positive pressure lumen 1320b occluding fluid communication to the liquid reservoir open to the atmosphere 1310 (without the plunger 1380a radially compressing the positive pressure lumen 1320b). Subsequently, with continued advancement when the single unitary device is in a fully displaced state the lead in (e.g., pin) further radially compresses the positive pressure lumen 1320b and also the plunger 1380a radially compresses the positive pressure lumen 1320b reducing the internal volume displacing the fluid collected therein thereby creating the positive pressure pulse. During the vacuum pressure interval, the single unitary device is retracted to the position of partial (initial) displacement so that the positive pressure pulse generator mechanism (e.g., plunger 1330a) is no longer radially compressing the positive pressure lumen 1320b (allowing unrestricted passage therethrough). Irrespective of whether the liquid replenishing gating device is an independently controllable separate device from the positive pressure pulse generator mechanism 1380 (e.g., plunger 1380a) or integrated/combined into a single unitary device, fluid communication between the positive pressure lumen 1320b and the liquid reservoir open to the atmosphere 1310 is blocked or occluded during both the vacuum pressure interval and the positive pressure interval to allow build-up of the vacuum pressure and positive pressure, respectively.

Liquid replenishment (i.e., timing of the opening of the liquid replenishing gating device 1385) may occur on demand or periodically (e.g., once every N cycles, where each cycle include a positive pressure interval and a vacuum pressure interval and N is an integer greater than 0). Moreover, the timing operation may occur manually or be programmed using a controller having an associated processor and memory device.

It is desirable to initiate liquid replenishment less than every cycle because every time the fluid is moved or looped back there is a slight delay. Referring to FIGS. 20 & 21, with every occurrence of liquid replenishment (“Stage 3”) there occurs a change in slope of the next positive pressure (“Stage 1”) to allow for the pressure during the liquid replenishment (“Stage 3”) thereby reducing cycling speed or frequency. Thus, there is a balance between sacrifice in cycling speed and ensuring the collected fluid is free from gas purged into the vacuum reservoir. Such balance is preferable when liquid replenishment does not occur with every cycle. That is, liquid replenishment most preferably occurs once every N cycles, where N is a positive integer greater than 1.

FIGS. 20-22 represent several example pressure vs. time graphical representations depicting the timing sequence for replenishing or restoring liquid to the active liquid channel of the hybrid cyclic aspiration system to compensate for that depleted aspirated liquid collected in the vacuum collection vessel 1305a in response to the cyclic aspiration pressure waveform. In the example of FIG. 20, replenishment of the depleted aspirated liquid is programmed to occur every cycle (i.e., where each cycle includes a positive pressure interval and a vacuum pressure interval). Stage #1 (denoted by the solid line) represents the creation of the positive pressure pulse (e.g., extension/advancement of the plunger 1380a by the actuator 1380b externally radially compressing the flexi section of the positive pressure lumen 1320b reducing the internal volume and displacing the collected liquid therein thereby creating the positive pressure pulse). In advance of creating the positive pressure pulse, the liquid replenishing gating device 1385 and the vacuum pressure gating device 1330 are both in a closed state preventing passage therethrough of the vacuum pressure generated by the vacuum pump 1305 to allow build-up of the positive pressure pulse in the positive pressure lumen 1320b. Next, in stage #2 (denoted by the dotted line) while the liquid replenishing gating device 1385 remains closed the vacuum pressure gating device 1330 transitions to an open state allowing unrestricted passage therethrough of the vacuum pressure generated by the vacuum pump 1305 and the plunger 1380a is retracted by the actuator 1380b. The vacuum pressure gating device 1330 is maintained in the open state and the plunger 1380a remains in a retracted state during a liquid replenishment interval in stage #3 (denoted by the dashed-dot line) wherein the liquid replenishing gating device 1385 is transitioned to an open state allowing passage of the liquid from the liquid reservoir open to the atmosphere 1310 into the positive pressure lumen 1320b. It is during stage #3 that liquid is restored to the inlet tubing compensating for the depleted aspirated liquid collected in the vacuum collection vessel 1305a. This 3-stage process repeats with replenishing (i.e., stage #3) the liquid in the cyclic aspiration system occurring each cycle (i.e., including a positive pressure interval and a vacuum pressure interval). Replenishment or restoration of the liquid to the system occurs once every N cycles, where N is an integer greater than 0. By way of illustrative example in FIG. 21, N is 3, with stage #3 liquid replenishment occurring every third cycle. The examples in FIGS. 20 & 21 depict a relatively brief period or duration of stage #3 in which of the liquid replenishment gating device 1385 is in an open state. In such relatively “brief” period of liquid replenishment the liquid replenishing gating device 1385 remains open for only that period during which pressure continuously rises, i.e., the pressure is not sustained at a plateau. It is also contemplated for the interval of liquid replenishment (stage #3) to be for a sustained period of time wherein the liquid replenishing gating device 1385 remains in an open state for an extended duration beyond that of the relatively brief duration of FIG. 21 during which the pressure remains at a plateau. FIG. 22 is an illustrative example of a sustained period of replenishment (i.e., longer in comparison to the relatively brief period or duration in the example in FIG. 21) occurring once every three cycles (e.g., N=3). Sustained duration of the replenishment stage at the pressure plateau provides extended “down time” to purge at will the gas from a portion of the line after a period of cycling by introducing a fresh volume of saline into the system.

These are mere illustrative examples of the sequencing operation of: the positive pressure pulse generator mechanism 1380, the vacuum pressure gating device 1330, and the liquid replenishing gating device 1385 periodically replenishing the liquid in the cyclic aspiration system. As previously mentioned, replenishment of the liquid to the cyclic aspiration system may occur manually on demand or preprogrammed to occur automatically every N cycles, wherein each cycle includes a positive pressure pulse interval and a vacuum pressure interval with N being an integer greater than 0. Furthermore, the duration or period of replenishment (i.e., liquid replenishing gating device 1385 while in an open state) may be selected or adjusted, as desired.

Maintaining a desired volume of liquid in the cyclic aspiration system by replenishing the depleted aspirated liquid minimizes the formation of new bubbles thereby optimizing continuity in the cyclic aspiration pressure waveform and efficiency in the capture of clots. The desired volume of liquid to be maintained in the cyclic aspiration system may be dependent on any number of different factors, e.g., the inner diameter and/or length of the tubes.

If not refilled, over time, the liquid in the liquid reservoir open to the atmosphere 1310 serving as the source for replenishing or restoring the liquid in the cyclic aspiration system will become exhausted. In the bi-directional (non-continuous loop) hybrid cyclic aspiration system described above in FIG. 3A, automatized self-prepping of the liquid reservoir with new (i.e., fresh, non-recirculated, or not previously aspirated from the cyclic aspiration system) liquid (e.g., saline) may be accomplished using one of the exemplary systems in FIGS. 5A-5C. Presumably, any of the systems in FIGS. 5A-5C could also be used to replenish the liquid reservoir with new saline undertaking the time-consuming sequence of operations as described in detail above. The unidirectional continuous loop hybrid cyclic aspiration system in accordance with the disclosure presents a streamlined process for refilling the liquid reservoir open to the atmosphere 1310 to that of a single operation, i.e., controlling a single gating device (i.e., liquid refilling gating device) 1390). Forming part of the unidirectional continuous loop, the liquid refilling gating device 1390 is interposed between the vacuum collection vessel 1305a and the liquid reservoir open to the atmosphere 1310. Instead of introducing new (i.e., fresh, non-recirculated, or not previously aspirated from the cyclic aspiration system) liquid (e.g., saline) (as in the automatized self-prepping systems in the examples of FIGS. 5A-5C), the unidirectional continuous loop hybrid cyclic aspiration system refills the liquid reservoir open to the atmosphere 1310 by recirculating the aspirated collected liquid along with the formed bubbles present therein stored in the vacuum collection vessel 1305a. Furthermore, since the liquid reservoir open to the atmosphere 1310 is replenished using the aspirated collected liquid stored in the vacuum collection vessel 1305a the need to empty that container may be eliminated altogether or less frequently.

In operation, during the production of the cyclic aspiration pressure waveform, the liquid refilling gating device 1390 remains closed preventing passage of the aspirated collected liquid together with any formed bubbles present therein stored in the vacuum collection vessel 1305a into the liquid reservoir open to the atmosphere 1310. Prior to initiating refill of the liquid reservoir open to the atmosphere 1310 (i.e., opening the liquid refilling gating device 1390), the vacuum pump is either turned off completely or the vacuum pressure produced is sufficiently reduced in intensity to prevent unintentional aspiration of the liquid from the liquid reservoir open to the atmosphere 1310 upstream into the vacuum collection vessel 1305a via the liquid refilling gating device 1390 while in an open state. Regardless of whether the vacuum pump is turned off completely or merely reduced in intensity during the refilling stage there is no cycling (i.e., no producing of the cyclic aspiration pressure waveform). Refilling of the liquid reservoir open to the atmosphere 1310 may occur either manually on demand or programmed to occur periodically (i.e., once every N cycles, wherein each cycle includes a positive pressure interval and a negative pressure interval with N being an integer greater than zero). Since the vacuum pump 1305 must be either turned off or the vacuum pressure being generated reduced in intensity, preferably the liquid refilling interval does not occur every cycle. While the liquid refilling gating device 1390 is in an open state the aspirated collected fluid along with the formed bubbles present therein from the vacuum collection vessel 1305a passes into the liquid reservoir open to the atmosphere 1310. Since the liquid reservoir 1310 is open to the atmosphere the formed bubbles present in the aspirated collected liquid received from the vacuum collection vessel 1305a automatically escape into the atmosphere so what remains is the degassed aspirated liquid (i.e., free from the formed bubbles present therein) to be recirculated back into the active liquid channel of the cyclic aspiration system during the next liquid replenishing interval (i.e., opening of the liquid replenishing gating device 1385), as described earlier. Thus, refilling of the liquid in the liquid reservoir open to the atmosphere 1310 is streamlined to operation of a single gating device using recirculated degassed aspirated liquid rather than having to introduce new (i.e., fresh, non-recirculated, or not previously aspirated from the cyclic aspiration system) liquid. As mentioned previously, the recirculation of the aspirated collected fluid from the vacuum collection vessel 1305a also advantageously eliminates altogether or reduces in frequency having to empty the container.

Operation in refilling the liquid reservoir open to the atmosphere 1310 with liquid will now be described. First, the vacuum pump 1305 is either turned off or reduced in level of intensity of the vacuum pressure produced (while the vacuum pressure gating device 1330 and liquid replenishing gating device 1385 are closed with the positive pressure pulse generator mechanism (e.g., plunger 1380a) retracted). As mentioned earlier, turning off completely or reducing in intensity the vacuum pressure generated by the vacuum pump 1305 prevents unintentional upstream drawing of the liquid from the liquid reservoir open to the atmosphere 1310 into the vacuum collection vessel 1305a when the liquid refilling gating device 1390 is in an open state. Thereafter, the liquid refilling gating device 1390 is transitioned to an open state allowing passage therethrough of the aspirated collected liquid along with the formed bubbles present therein from the vacuum collection vessel 1305a into the liquid reservoir open to the atmosphere 1310. On demand or periodically the captured clots in the vacuum collection vessel 1305a may be removed. However, any of the captured clots remaining in the vacuum collection vessel 1305a are prevented from exiting via a filter associated therewith. Therefore, when the liquid refilling gating device 1390 is transitioned to the open state, only the aspirated collected liquid along with the formed bubbles present therein stored in the vacuum collection vessel 1305a travel into the liquid reservoir open to the atmosphere 1310. As the aspirated liquid is received in the liquid reservoir 1310 the formed bubbles present therein escape into the atmosphere so only degassed aspirated liquid (i.e., free of the formed bubbles) remains. With the liquid reservoir open to the atmosphere 1310 now refilled with the degassed aspirated liquid, the vacuum pump 1305 is either turned on or increased in intensity to resume production of the cyclic aspiration pressure waveform (i.e., cyclic intermittent intervals of positive pressure and vacuum pressure), as described above in stages #1 & #2 of FIGS. 20-22. During the next replenishment interval (stage #3—i.e., the liquid replenishing gating device 1385 transitioned to the open state) the degassed aspirated liquid in the liquid reservoir open to the atmosphere 1310 is recirculated back into the cyclic aspiration system.

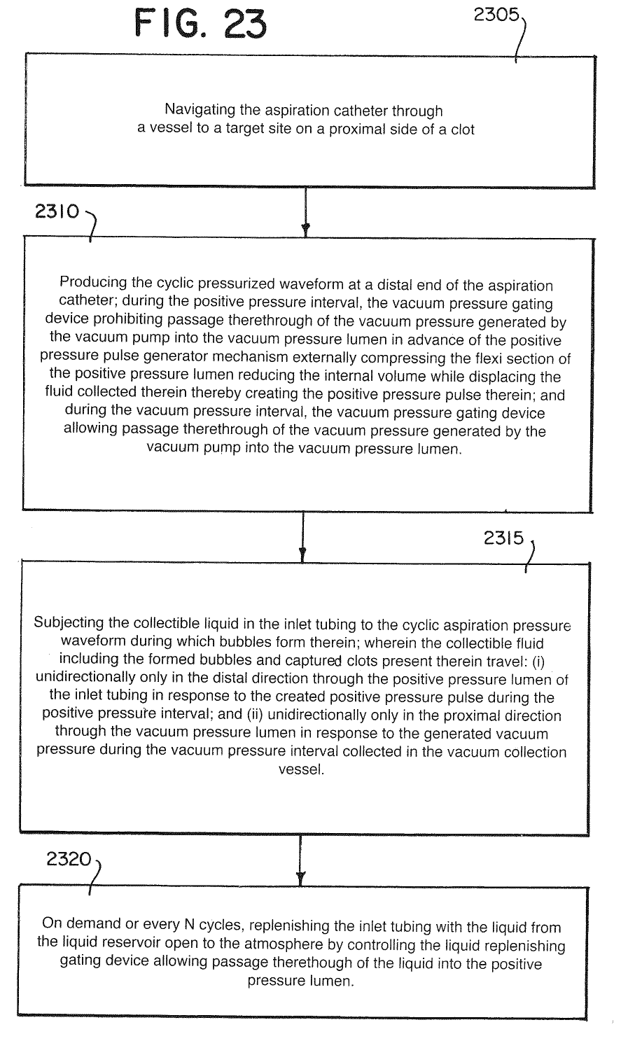

In operation, FIG. 23 is an exemplary flow chart of the unidirectional continuous loop hybrid cyclic aspiration system in accordance with the present disclosure. In step 2305 the aspiration catheter is navigated through a vessel to a target site on a proximal side of a clot to be captured or ingested. Next, the cyclic pressurized waveform is produced at a distal end/tip of the aspiration catheter 123 in step 2310. Specifically, during the positive pressure interval, the vacuum pressure gating device 1330 is transitioned to the closed state prohibiting passage therethrough of the vacuum pressure generated by the vacuum pump 1305 into the vacuum pressure lumen 1320a to allow build-up of the to be created positive pressure pulse thereafter. Then, the positive pressure pulse is created by the positive pressure pulse generator mechanism 1380 (e.g., plunger 1380a advanced/extended via actuator 1380b) externally compressing the flexi section of the positive pressure lumen 1320b reducing the internal volume while displacing the fluid collected therein. During the vacuum pressure interval, the vacuum pressure gating device 1330 is transitioned to an open state allowing unrestricted passage therethrough of the vacuum pressure generated by the vacuum pump 1305 into the vacuum pressure lumen 1320a (with the plunger 1380a in a retracted position). In step 2315 the collected liquid in the active liquid channel of the inlet tubing 1320 is subjected to the cyclic aspiration pressure waveform causing the formation of bubbles therein. In the active liquid channel the collected fluid (i.e., collected liquid (e.g., saline and/or blood) together with the formed bubbles and captured clots present therein) travels: (i) unidirectionally only in the distal direction through the positive pressure lumen 1320b in response to the created positive pressure pulse during the positive pressure interval; and (ii) unidirectionally only in the proximal direction through the vacuum pressure lumen 1320a during the vacuum pressure interval to be collected in the vacuum collection vessel 1305a. On demand or every N cycles (where each cycle includes a positive pressure interval and a vacuum pressure interval with N being an integer greater than 0), in step 2320 the active liquid channel of the inlet tubing 1320 depleted of the aspirated collected liquid (e.g., saline and/or blood) is replenished using the liquid from the liquid reservoir open to the atmosphere 1310 by controlling (e.g., opening) the liquid replenishing gating device 1385 allowing passage therethrough into the positive pressure lumen 1320b.

Over time, operation of the unidirectional continuous loop hybrid cyclic aspiration system depletes the liquid present in the liquid reservoir open to the atmosphere 1310. The flow chart in FIG. 24 represents the steps during the refilling operation of the liquid reservoir open to the atmosphere 1310. In step 2405 the vacuum pump 1305 is either turned off completely or reduced in intensity of the vacuum pressure generated to prevent the aspiration of the liquid from the liquid reservoir open to the atmosphere 1310 into the vacuum collection vessel 1305a prior to the liquid refilling gating device 1390 transitioning to an open state. Next, in step 2410 the liquid refilling gating device 1390 is transitioned to an open state allowing unrestricted passage therethrough of the collected liquid (e.g., saline and/or blood) together with the formed bubbles present therein) to the liquid reservoir open to the atmosphere 1310 from the vacuum collection vessel 1305a. Upon being received in the liquid reservoir open to the atmosphere 1310, in step 2415 the formed bubbles present in the collected liquid (e.g., saline and/or blood) escape into the atmosphere leaving behind only the degassed aspirated liquid (i.e., free of the formed bubbles). In the next replenishing operation for restoring liquid to the inlet tubing (i.e., opening of the liquid replenishing gating device 1385) in step 2420 of FIG. 23 described above, the degassed aspirated liquid present in the refilled liquid reservoir open to the atmosphere 1310 is recirculated into the positive pressure lumen 1320b restoring the depleted aspirated liquid for continuing operation of the unidirectional continuous loop hybrid cyclic aspiration system.

Any of the gating devices are transitionable between a closed state preventing, blocking, or occluding passage therethrough and an open state allowing unrestricted passage therethrough (e.g., a valve, pinch valve, pin, plunger, etc. and associated actuator, solenoid, reciprocating motor, or electromagnet) as described herein.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

Aspects of the present disclosure are also provided by the following numbered Clauses:

### Clause 1

A unidirectional continuous loop hybrid cyclic aspiration system producing an associated cyclic aspiration pressure waveform of intermittent cyclic intervals including a vacuum pressure interval at a vacuum pressure below atmospheric pressure and a positive pressure interval at a positive pressure higher than the vacuum pressure, the cyclic aspiration system comprising: a vacuum pump (1305) generating the vacuum pressure; the vacuum pump (1305) having associated therewith a vacuum collection vessel (1305a); a liquid reservoir open to atmosphere (1310) connected in fluid communication with the vacuum collection vessel (1305a) and filled with a liquid; inlet tubing (1320) having defined therein a vacuum pressure lumen (1320a) and a positive pressure lumen (1320b); the vacuum pressure lumen (1320a) connected in fluid communication distally to the vacuum pump (1305) and the positive pressure lumen (1320b) connected in fluid communication distally to the liquid reservoir open to the atmosphere (1310); and the vacuum collection vessel (1305a) receiving via the vacuum pressure lumen (1320a) aspirated collectable liquid during the vacuum pressure interval of the cyclic aspiration pressure waveform; an aspiration catheter (1323) connected via a catheter hub (1327) in fluid communication to the inlet tubing (1320); and a positive pressure pulse generator mechanism (1380) disposed externally of the positive pressure lumen (1320b) and transitionable between an extended state and a retracted state; wherein in the extended state the positive pressure pulse generator mechanism (1380) radially compressing the positive pressure lumen (1320b) reducing an internal volume while displacing the collectable fluid therein creating a positive pressure pulse; and when the positive pressure pulse generator mechanism (1380) is in the retracted state, the positive pressure lumen (1320b) automatically revertible to an original radially non-compressed state; a vacuum pressure gating device (1330) arranged externally of the vacuum pressure lumen (1320a) and transitionable between a restricted state and a non-restricted state; while in the non-restricted state the vacuum pressure gating device (1330) allowing unrestricted passage therethrough into the vacuum pressure lumen 1320a the vacuum pressure generated by the vacuum pump (1305) representing the vacuum pressure interval of the cyclic aspiration pressure waveform and while in the extended state prohibiting passage therethrough into the vacuum pressure lumen (1320a) of the vacuum pressure generated by the vacuum pump (1305) in advance of the positive pressure pulse generator mechanism (1380) in the extended state creating the positive pressure pulse representing the positive pressure interval of the cyclic aspiration pressure waveform; and a liquid replenishing gating device (1385) disposed distally of the liquid reservoir open to the atmosphere (1310) controlling passage therethrough of the liquid from the liquid reservoir open to the atmosphere (1310) into the positive pressure lumen (1320b) replenishing the cyclic aspiration system in response to the aspirated collectable fluid received in the vacuum collection vessel (1305a); wherein travel of the collectable fluid is: (i) unidirectionally only in a proximal direction through the vacuum pressure lumen (1320a) and receivable in the vacuum collection vessel (1305a) during the vacuum pressure interval of the cyclic aspiration pressure waveform; and (ii) unidirectionally only in a distal direction through the positive pressure lumen (1320b) during the positive pressure interval of the cyclic aspiration pressure waveform.

### Clause 2

The system of Clause 1, wherein the liquid replenishing gating device (1385) allows passage therethrough of the liquid from the liquid reservoir open to atmospheric pressure (1310) into the positive pressure lumen (1320b) either on demand or once every N cycles, wherein each cycle includes the positive pressure interval and the vacuum pressure interval with N being a positive integer greater than 0.

### Clause 3

The system of any of Clauses 1 through 2, further comprising a liquid refilling gating device (1390) controlling passage into the liquid reservoir open to the atmosphere (1310) of the collectable fluid from the vacuum collection vessel (1305a); the liquid refilling gating device (1390) only allowing flow therethrough when the vacuum pressure pump (1305) is either turned off or the vacuum pressure being produced is sufficiently reduced to prevent passage of the liquid from the liquid reservoir open to the reservoir (1310) into the vacuum collection vessel (1305a) when the liquid refilling gating device (1390) is in an open state.

### Clause 4

The system of any of Clauses 1 through 3, wherein the inlet tubing (1320) comprises: (i) a single inlet tube having two separate lumen defined therein representing respectively the vacuum pressure lumen (1320a) and the positive pressure lumen (1320b); or (ii) a first inlet tube having a single lumen defined therein representing the vacuum pressure lumen (1320a) and a second inlet tube having a single lumen defined therein representing the positive pressure lumen (1320b), wherein the first inlet tube is separate from the second inlet tube.

### Clause 5

The system of Clause 4, wherein the inlet tubing (1320) comprises the single inlet tube and the two separate lumen are arranged: side-by-side parallel to one another in a longitudinal direction, concentrically, or eccentrically.

### Clause 6

The system of any of Clauses 1 through 5, wherein a clearance space (1321) is provided in a distal section of the inlet tubing (1320) and/or the catheter hub (1327); the clearance space (1321) allowing continuous travel of the collectable fluid in the inlet tubing (1320) from a distal end of the positive pressure lumen (1320b) into a distal end of the vacuum pressure lumen (1320a) while transitioning from the positive pressure interval to the vacuum pressure interval.

### Clause 7

A method for capturing a clot using a unidirectional continuous loop hybrid cyclic aspiration system (1300) producing an associated cyclic aspiration pressure waveform of intermittent cyclic intervals of vacuum pressure below atmospheric pressure and a positive pressure higher than the vacuum pressure, the cyclic aspiration system including: a vacuum pump (1305) generating the vacuum pressure; the vacuum pump (1305) having associated therewith a vacuum collection vessel (1305a); a liquid reservoir open to atmosphere (1310) connected in fluid communication with the vacuum collection vessel (1305a) and filled with a liquid; inlet tubing (1320) having defined therein a vacuum pressure lumen (1320a) and a positive pressure lumen (1320b); the vacuum pressure lumen (1320a) connected in fluid communication distally to the vacuum pump (1305) and the positive pressure lumen (1320b) connected in fluid communication distally to the liquid reservoir open to the atmosphere (1310); and the vacuum collection vessel (1305a) receiving via the vacuum pressure lumen (1320a) aspirated collectable liquid during the vacuum pressure interval of the cyclic aspiration pressure waveform; an aspiration catheter (1323) connected via a catheter hub (1327) in fluid communication to the inlet tubing (1320); and a positive pressure pulse generator mechanism (1380) disposed externally of the positive pressure lumen (1320b) and transitionable between an extended state and a retracted state; wherein in the extended state the positive pressure pulse generator mechanism (1380) radially compressing the positive pressure lumen (1320b) reducing an internal volume while displacing the collectable fluid therein creating a positive pressure pulse; and when the positive pressure pulse generator mechanism (1380) is in the retracted state, the positive pressure lumen (1320b) automatically revertible to an original radially non-compressed state; a vacuum pressure gating device (1330) arranged externally of the vacuum pressure lumen (1320a) and transitionable between a restricted state and a non-restricted state; while in the non-restricted state the vacuum pressure gating device (1330) allowing unrestricted passage therethrough into the vacuum pressure lumen 1320a the vacuum pressure generated by the vacuum pump (1305) representing the vacuum pressure interval of the cyclic aspiration pressure waveform and while in the extended state prohibiting passage therethrough into the vacuum pressure lumen (1320a) of the vacuum pressure generated by the vacuum pump (1305) in advance of the positive pressure pulse generator mechanism (1380) in the extended state creating the positive pressure pulse representing the positive pressure interval of the cyclic aspiration pressure waveform; and a liquid replenishing gating device (1385) disposed distally of the liquid reservoir open to the atmosphere (1310) controlling passage therethrough of the liquid from the liquid reservoir open to the atmosphere (1310) into the positive pressure lumen (1320b) replenishing the cyclic aspiration system in response to the aspirated collectable fluid received in the vacuum collection vessel (1305a); wherein travel of the collectable fluid is: (i) unidirectionally only in a proximal direction through the vacuum pressure lumen (1320a) and receivable in the vacuum collection vessel (1305a) during the vacuum pressure interval of the cyclic aspiration pressure waveform; and (ii) unidirectionally only in a distal direction through the positive pressure lumen (1320b) during the positive pressure interval of the cyclic aspiration pressure waveform; the method comprising the steps of: navigating the aspiration catheter (1323) through a vessel to a target site on a proximal side of the clot; producing the cyclic aspiration pressure waveform at a distal tip of the aspiration catheter (1323); and during the positive pressure interval, the vacuum pressure gating device (1330) prohibiting passage therethrough of the vacuum pressure generated by the vacuum pump (1305) in advance of the positive pressure pulse generator mechanism (1380) externally radially compressing the positive pressure lumen (1320b) reducing the internal volume while displacing the fluid collected therein thereby creating the positive pressure pulse; and during the vacuum pressure interval, the vacuum pressure gating device (1330) allowing unrestricted passage therethrough of the vacuum pressure generated by the vacuum pump (1305); subjecting the collectable fluid in the inlet tubing (1320) to the cyclic aspiration pressure waveform during which bubbles form therein; the collectable fluid traveling unidirectionally only in the distal direction through the positive pressure lumen (1320b) in response to the created positive pressure pulse during the positive pressure interval; and the collectable fluid traveling unidirectionally only in the proximal direction through the vacuum pressure lumen (1320a) and received in the vacuum collection vessel (1305a) during the vacuum pressure interval; and replenishing the inlet tubing (1320) with the liquid from the liquid reservoir open to the atmosphere (1310) by controlling the liquid replenishing gating device (1385) allowing passage therethrough of the liquid into the positive pressure lumen (1320b).

### Clause 8

The method of Clause 7, wherein the replenishing step occurs on demand or every N cycles of the cyclic aspiration pressure waveform, wherein each cycle includes the positive pressure interval and the vacuum pressure interval with N being a positive integer greater than 0.

### Clause 9

The method of any of Clauses 7 through 8, further comprising the step of refilling the liquid in the liquid reservoir open to the atmosphere (1310); wherein the refilling step comprises the steps of: turning off or sufficiently reducing in level the vacuum pressure generated by the vacuum pump; controlling the liquid refilling gating device (1390) allowing unrestricted passage therethrough of the collectable fluid stored in the vacuum collection vessel (1305a) into the liquid reservoir open to the atmosphere (1310); degassing via escaping into the atmosphere formed bubbles present in the collected fluid from the vacuum collection vessel (1305a) when received by the liquid reservoir open to the atmosphere (1310) leaving therein degassed aspirated liquid; resuming the producing of the cyclic aspiration pressure waveform by either turning back on or restoring to a previous level prior to being reduced the vacuum pressure generated by the vacuum pump (1305); and while the liquid replenishing gating device (1385) is allowing unrestricted passage therethrough, recirculating the degassed aspirated liquid from the liquid reservoir open to the atmosphere (1310) into the positive pressure lumen (1320b).

### Clause 10

The method of any of Clauses 7 through 9, wherein the producing the cyclic aspiration pressure waveform comprises independently controlling each of the vacuum pressure gating device (1330), the liquid replenishing gating device (1385), and the positive pressure pulse generator mechanism (1380); wherein during the positive pressure interval: the vacuum pressure gating device (1330) prohibiting passage therethrough of the vacuum pressure and the liquid replenishing gating device (1385) prohibiting passage therethrough of the liquid from the liquid reservoir open to the atmosphere (1310) while the positive pressure pulse generator mechanism (1380) in the extended state is radially compressing the positive pressure lumen (1320b) reducing the internal volume displacing the collectable fluid therein and creating the positive pressure pulse; wherein during the vacuum pressure interval, the vacuum pressure gating device (1330) allowing unrestricted passage therethrough of the vacuum pressure and the liquid replenishing gating device (1385) prohibiting passage therethrough while the positive pressure pulse generator mechanism (1380) is in the retracted state.

### Clause 11

The method of any of Clauses 7 through 10, wherein during the replenishing step, the liquid replenishing gating device (1385) allowing passage therethrough of the liquid from the liquid reservoir open to atmospheric pressure (1310) into the positive pressure lumen (1320b), and the vacuum pressure gating device (1330) allowing passage therethrough of the vacuum pressure generated by the vacuum pump (1305).

### Clause 12

The method of any of Clauses 7 through 11, wherein the inlet tubing (1320) comprises: (i) a single inlet tube having two separate lumen defined therein representing respectively the vacuum pressure lumen (1320a) and the positive pressure lumen (1320b); or (ii) a first inlet tube having defined therein a single lumen representing the vacuum pressure lumen (1320a) and a second inlet tube having defined therein a single lumen representing the positive pressure lumen (1320b), wherein the first inlet tube is separate from the second inlet tube.

### Clause 13

The method of Clause 12, wherein the inlet tubing (1320) comprises the single inlet tube and the two separate lumen defined therein are arranged: side-by-side parallel to one another in a longitudinal direction, concentrically, or eccentrically.

### Clause 14

The method of Clause 7, wherein a clearance space (1321) is provided in a distal section of the inlet tubing (1320) and/or the catheter hub (1327); the clearance space (1321) allowing continuous loop travel of the collectable fluid in the inlet tubing (1320) from a distal end of the positive pressure lumen (1320b) into a distal end of the vacuum pressure lumen (1320a) while transitioning from the positive pressure interval to the vacuum pressure interval.

The descriptions contained herein are examples and are not intended in any way to limit the scope of the present disclosure. As described herein, the present disclosure contemplates many variations and modifications of a unidirectional continuous loop hybrid cyclic aspiration system producing a cyclic aspiration pressure waveform of intermittent cyclic intervals of vacuum pressure below atmospheric pressure and positive pressure higher than vacuum pressure (higher than vacuum pressure, possibly higher than atmospheric pressure) using as few active components as possible with an associated maximized response time to attain maximum cycling frequency. In connection therewith the present disclosure further contemplates minimizing dampening or decay of the produced cyclic aspiration pressure waveform by replenishing depleted aspirated liquid from the inlet tubing with liquid from the liquid reservoir open to the atmosphere as well as refilling of the liquid in the liquid reservoir open to the atmosphere by recirculating the aspirated collected liquid. Modifications and variations apparent to those having skilled in the pertinent art according to the teachings of this disclosure are intended to be within the scope of the claims which follow.

What is claimed is:

1. A unidirectional continuous loop hybrid cyclic aspiration system producing an associated cyclic aspiration pressure waveform of intermittent cyclic intervals including a vacuum pressure interval at a vacuum pressure below atmospheric pressure and a positive pressure interval at a positive pressure higher than the vacuum pressure, the cyclic aspiration system comprising:
a vacuum pump generating the vacuum pressure; the vacuum pump having associated therewith a vacuum collection vessel;
a liquid reservoir open to atmosphere connected in fluid communication with the vacuum collection vessel and filled with a liquid;
inlet tubing having defined therein a vacuum pressure lumen and a positive pressure lumen; the vacuum pressure lumen connected in fluid communication distally to the vacuum pump and the positive pressure lumen connected in fluid communication distally to the liquid reservoir open to the atmosphere; and the vacuum collection vessel receiving via the vacuum pressure lumen aspirated collectable liquid during the vacuum pressure interval of the cyclic aspiration pressure waveform;
an aspiration catheter connected via a catheter hub in fluid communication to the inlet tubing; and
a positive pressure pulse generator mechanism disposed externally of the positive pressure lumen and transitionable between an extended state and a retracted state; wherein in the extended state the positive pressure pulse generator mechanism radially compressing the positive pressure lumen reducing an internal volume while displacing the collectable fluid therein creating a positive pressure pulse; and when the positive pressure pulse generator mechanism is in the retracted state, the positive pressure lumen automatically revertible to an original radially non-compressed state;
a vacuum pressure gating device arranged externally of the vacuum pressure lumen and transitionable between a restricted state and a non-restricted state; while in the non-restricted state the vacuum pressure gating device allowing unrestricted passage therethrough into the vacuum pressure lumen the vacuum pressure generated by the vacuum pump representing the vacuum pressure interval of the cyclic aspiration pressure waveform and while in the extended state prohibiting passage therethrough into the vacuum pressure lumen of the vacuum pressure generated by the vacuum pump in advance of the positive pressure pulse generator mechanism in the extended state creating the positive pressure pulse representing the positive pressure interval of the cyclic aspiration pressure waveform; and
a liquid replenishing gating device disposed distally of the liquid reservoir open to the atmosphere controlling passage therethrough of the liquid from the liquid reservoir open to the atmosphere into the positive pressure lumen replenishing the cyclic aspiration system in response to the aspirated collectable fluid received in the vacuum collection vessel;
wherein travel of the collectable fluid is: (i) unidirectionally only in a proximal direction through the vacuum pressure lumen and receivable in the vacuum collection vessel during the vacuum pressure interval of the cyclic aspiration pressure waveform; and (ii) unidirectionally only in a distal direction through the positive pressure lumen during the positive pressure interval of the cyclic aspiration pressure waveform.

2. The system of claim 1, wherein the liquid replenishing gating device allows passage therethrough of the liquid from the liquid reservoir open to atmospheric pressure into the positive pressure lumen either on demand or once every N cycles, wherein each cycle includes the positive pressure interval and the vacuum pressure interval with N being a positive integer greater than 0.

3. The system of claim 2, further comprising a liquid refilling gating device controlling passage into the liquid reservoir open to the atmosphere of the collectable fluid from the vacuum collection vessel; the liquid refilling gating device only allowing flow therethrough when the vacuum pressure pump is either turned off or the vacuum pressure being produced is sufficiently reduced to prevent passage of the liquid from the liquid reservoir open to the reservoir into the vacuum collection vessel when the liquid refilling gating device is in an open state.

4. The system of claim 1, wherein the inlet tubing comprises: (i) a single inlet tube having two separate lumen defined therein representing respectively the vacuum pressure lumen and the positive pressure lumen; or (ii) a first inlet tube having a single lumen defined therein representing the vacuum pressure lumen and a second inlet tube having a single lumen defined therein representing the positive pressure lumen, wherein the first inlet tube is separate from the second inlet tube.

5. The system of claim 4, wherein the inlet tubing comprises the single inlet tube and the two separate lumens are arranged: side-by-side parallel to one another in a longitudinal direction, concentrically, or eccentrically.

6. The system of claim 1, wherein a clearance space is provided in a distal section of the inlet tubing and/or the catheter hub; the clearance space allowing continuous travel of the collectable fluid in the inlet tubing from a distal end of the positive pressure lumen into a distal end of the vacuum pressure lumen while transitioning from the positive pressure interval to the vacuum pressure interval.

7. A method for capturing a clot using a unidirectional continuous loop hybrid cyclic aspiration system producing an associated cyclic aspiration pressure waveform of intermittent cyclic intervals of vacuum pressure below atmospheric pressure and a positive pressure higher than the vacuum pressure,
the cyclic aspiration system including: a vacuum pump generating the vacuum pressure; the vacuum pump having associated therewith a vacuum collection vessel; a liquid reservoir open to atmosphere connected in fluid communication with the vacuum collection vessel and filled with a liquid; inlet tubing having defined therein a vacuum pressure lumen and a positive pressure lumen; the vacuum pressure lumen connected in fluid communication distally to the vacuum pump and the positive pressure lumen connected in fluid communication distally to the liquid reservoir open to the atmosphere; and the vacuum collection vessel receiving via the vacuum pressure lumen aspirated collectable liquid during the vacuum pressure interval of the cyclic aspiration pressure waveform; an aspiration catheter connected via a catheter hub in fluid communication to the inlet tubing; and a positive pressure pulse generator mechanism disposed externally of the positive pressure lumen and transitionable between an extended state and a retracted state;
wherein in the extended state the positive pressure pulse generator mechanism radially compressing the positive pressure lumen reducing an internal volume while displacing the collectable fluid therein creating a positive pressure pulse; and when the positive pressure pulse generator mechanism is in the retracted state, the positive pressure lumen automatically revertible to an original radially non-compressed state; a vacuum pressure gating device arranged externally of the vacuum pressure lumen and transitionable between a restricted state and a non-restricted state; while in the non-restricted state the vacuum pressure gating device allowing unrestricted passage therethrough into the vacuum pressure lumen, the vacuum pressure generated by the vacuum pump representing the vacuum pressure interval of the cyclic aspiration pressure waveform and while in the extended state prohibiting passage therethrough into the vacuum pressure lumen of the vacuum pressure generated by the vacuum pump in advance of the positive pressure pulse generator mechanism in the extended state creating the positive pressure pulse representing the positive pressure interval of the cyclic aspiration pressure waveform; and a liquid replenishing gating device disposed distally of the liquid reservoir open to the atmosphere controlling passage therethrough of the liquid from the liquid reservoir open to the atmosphere into the positive pressure lumen replenishing the cyclic aspiration system in response to the aspirated collectable fluid received in the vacuum collection vessel;
wherein travel of the collectable fluid is: (i) unidirectionally only in a proximal direction through the vacuum pressure lumen and receivable in the vacuum collection vessel during the vacuum pressure interval of the cyclic aspiration pressure waveform; and (ii) unidirectionally only in a distal direction through the positive pressure lumen during the positive pressure interval of the cyclic aspiration pressure waveform;
the method comprising the steps of:
navigating the aspiration catheter through a vessel to a target site on a proximal side of the clot; producing the cyclic aspiration pressure waveform at a distal tip of the aspiration catheter; and during the positive pressure interval, the vacuum pressure gating device prohibiting passage therethrough of the vacuum pressure generated by the vacuum pump in advance of the positive pressure pulse generator mechanism externally radially compressing the positive pressure lumen reducing the internal volume while displacing the fluid collected therein thereby creating the positive pressure pulse; and during the vacuum pressure interval, the vacuum pressure gating device allowing unrestricted passage therethrough of the vacuum pressure generated by the vacuum pump;
subjecting the collectable fluid in the inlet tubing to the cyclic aspiration pressure waveform during which bubbles form therein; the collectable fluid traveling unidirectionally only in the distal direction through the positive pressure lumen in response to the created positive pressure pulse during the positive pressure interval; and the collectable fluid traveling unidirectionally only in the proximal direction through the vacuum pressure lumen and received in the vacuum collection vessel during the vacuum pressure interval; and
replenishing the inlet tubing with the liquid from the liquid reservoir open to the atmosphere by controlling the liquid replenishing gating device allowing passage therethrough of the liquid into the positive pressure lumen.

8. The method of Clause 7, wherein the replenishing step occurs on demand or every N cycles of the cyclic aspiration pressure waveform, wherein each cycle includes the positive pressure interval and the vacuum pressure interval with N being a positive integer greater than 0.

9. The method of claim 7, further comprising the step of refilling the liquid in the liquid reservoir open to the atmosphere; wherein the refilling step comprises the steps of: turning off or sufficiently reducing in level the vacuum pressure generated by the vacuum pump; controlling the liquid refilling gating device allowing unrestricted passage therethrough of the collectable fluid stored in the vacuum collection vessel into the liquid reservoir open to the atmosphere; degassing via escaping into the atmosphere formed bubbles present in the collected fluid from the vacuum collection vessel when received by the liquid reservoir open to the atmosphere leaving therein degassed aspirated liquid; resuming the producing of the cyclic aspiration pressure waveform by either turning back on or restoring to a previous level prior to being reduced the vacuum pressure generated by the vacuum pump; and while the liquid replenishing gating device is allowing unrestricted passage therethrough, recirculating the degassed aspirated liquid from the liquid reservoir open to the atmosphere into the positive pressure lumen.

10. The method of claim 7, wherein the producing the cyclic aspiration pressure waveform comprises independently controlling each of the vacuum pressure gating device, the liquid replenishing gating device, and the positive pressure pulse generator mechanism; wherein during the positive pressure interval: the vacuum pressure gating device prohibiting passage therethrough of the vacuum pressure and the liquid replenishing gating device prohibiting passage therethrough of the liquid from the liquid reservoir open to the atmosphere while the positive pressure pulse generator mechanism in the extended state is radially compressing the positive pressure lumen reducing the internal volume displacing the collectable fluid therein and creating the positive pressure pulse; wherein during the vacuum pressure interval, the vacuum pressure gating device allowing unrestricted passage therethrough of the vacuum pressure and the liquid replenishing gating device prohibiting passage therethrough while the positive pressure pulse generator mechanism is in the retracted state.

11. The method of claim 7, wherein during the replenishing step, the liquid replenishing gating device allowing passage therethrough of the liquid from the liquid reservoir open to atmospheric pressure into the positive pressure lumen, and the vacuum pressure gating device allowing passage therethrough of the vacuum pressure generated by the vacuum pump.

12. The method of claim 7, wherein the inlet tubing comprises: (i) a single inlet tube having two separate lumen defined therein representing respectively the vacuum pressure lumen and the positive pressure lumen; or (ii) a first inlet tube having defined therein a single lumen representing the vacuum pressure lumen and a second inlet tube having defined therein a single lumen representing the positive pressure lumen, wherein the first inlet tube is separate from the second inlet tube.

13. The method of claim 12, wherein the inlet tubing comprises the single inlet tube and the two separate lumen defined therein are arranged: side-by-side parallel to one another in a longitudinal direction, concentrically, or eccentrically.

14. The method of claim 7, wherein a clearance space is provided in a distal section of the inlet tubing and/or the catheter hub; the clearance space allowing continuous loop travel of the collectable fluid in the inlet tubing from a distal end of the positive pressure lumen into a distal end of the vacuum pressure lumen while transitioning from the positive pressure interval to the vacuum pressure interval.
